(19) United States
(12) Patent Application Publication
(10) Pub. No.: US 2023/0357239 A1
(43) Pub. Date: Nov. 9, 2023

(54) PYRIDO[2,3-D]PYRIMIDIN-4-AMINES AS SOS1 INHIBITORS

(71) Applicant: Bayer Aktiengesellschaft, Leverkusen (DE)

(72) Inventors: Keith GRAHAM, Berlin (DE); Benjamin BADER, Berlin (DE); Jens SCHROEDER, Berlin (DE); Roman HILLIG, Berlin (DE); Hans BRIEM, Bremen (DE); Jeremie Xavier G. MORTIER, Bremen (DE); Felix PAPE, Berlin (DE); Steffen GRESSIES, Berlin (DE); Timo STELLFELD, Berlin (DE)

(21) Appl. No.: 18/245,594

(22) PCT Filed: Sep. 15, 2021
(86) PCT No.: PCT/EP2021/075316

§ 371 Date: Mar. 16, 2023

(30) Foreign Application Priority Data
Sep. 18, 2020 (EP) 20196818.7

(51) Int. Cl. C07D 471/04; C07D 519/00; A61P 35/00

(52) U.S. Cl. CPC C07D 471/04; C07D 519/00; A61P 35/00

(57) ABSTRACT

The present invention covers Pyrido[2,3-d]pyrimidin-4-amines compounds of general formula (I):

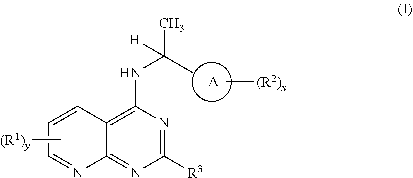

in which R¹, R², R³, A, x and y are as defined herein, methods of preparing said compounds, intermediate compounds useful for preparing said compounds, pharmaceutical compositions and combinations comprising said compounds and the use of said compounds for manufacturing pharmaceutical compositions for the treatment or prophylaxis of diseases, in particular of hyperproliferative disorders, as a sole agent or in combination with other active ingredients.

The present invention covers Pyrido[2,3-d]pyrimidin-4-amines compounds of general formula (I) as described and defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases, in particular of hyperproliferative disorders, as a sole agent or in combination with other active ingredients.

### BACKGROUND

The present invention covers Pyrido[2,3-d]pyrimidin-4-amines compounds of general formula (I)

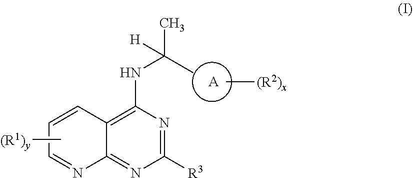

which inhibit the Ras-Sos1 interaction.

US 2011/0054173 A1 discloses certain 1- or 2-(4-(aryloxy)-phenyl)ethylamino-, oxy- or sulfanyl)pteridines and 1- or 2-(4-(heteroaryloxy)-phenyl)ethylamino-, oxy- or sulfanyl)pteridines and their use as agrochemicals and animal health products.

In the 2-position substituted quinazoline compounds are described e.g. in EP 0326328, EP 0326329, WO93/007124, WO2003/087098 and U.S. Pat. No. 5,236,925. These compounds are either not described as pharmaceutically active compounds or, if they are described as pharmacologically active compounds, they are described as compounds having affinity to the Epidermal Growth Factor Receptor (EGFR).

In the majority (45-100%) of patients receiving EGFR inhibitors skin toxicity is a class-specific side effect that is typically manifested as a papulopustular rash. The skin toxicity is related to the inhibition of EGFR in the skin, which is crucial for the normal development and physiology of the epidermis.

However, the state of the art does not describe:
the pyrido- and pyrrolopyrimidine compounds of general formula (I) of the present invention as described and defined herein, i.e. compounds having a pyrido- and pyrrolopyrimidine core which effectively and selectively inhibit the Ras-Sos1 interaction.

Ras proteins play an important role in human cancer. Mutations in Ras proteins can be found in 20-30% of all human tumors and are recognized as tumorigenic drivers especially in lung, colorectal and pancreatic cancers (Malumbres & Barbacid 2002 Nature Reviews Cancer, Pylayeva-Gupta et al. 2011 Nature Reviews Cancer). Three human Ras genes are known that encode four different Ras proteins of 21 kDa size: H-Ras, N-Ras, and two splice variants of K-Ras, namely K-Ras 4A and K-Ras-4B. All Ras isoforms are highly conserved within the GTP-binding domain and differ mainly in the hypervariable C-terminal region. The C-termini of the different Ras-isoforms are posttranslationally modified by lipidation (farnesylation, palmitoylation) to facilitate membrane anchorage. The localization of Ras-proteins at the cytoplasmic membrane provides vicinity to transmembrane growth receptors and has been shown to be essential for transmitting growth signals from extracellular growth factor binding to intracellular downstream pathways. A variety of upstream signals may activate Ras proteins depending on the cellular context, such as epidermal growth factor receptor (EGFR), platelet-derived growth factor receptor (PDGFR), nerve growth factor receptor (NGFR) and others. Activated Ras can signal through various downstream pathways, e.g. the Raf-MEK-ERK or the PI3K-PDK1-Akt pathways.

On the molecular level, Ras proteins function as molecular switches. By binding GTP and GDP they exist in an active (GTP-bound) and inactive (GDP-bound) state in the cell. Active GTP-loaded Ras recruits other proteins by binding of their cognate Ras-binding domains (RBDs) resulting in activation of the effector protein followed by downstream signalling events of diverse functions, e.g. cytoskeletal rearrangements or transcriptional activation. The activity status of Ras is tightly regulated by guanine nucleotide exchange factors (GEFs) and GTPase activating proteins (GAPs). GEFs function as activators of Ras by promoting the nucleotide exchange from GDP to GTP. GAPs deactivate Ras-GTP by catalyzing the hydrolysis of the bound GTP to GDP. In a cancer cell, point mutations, typically within the GTP-binding region at codon 12, eliminate the ability of RAS to efficiently hydrolyse bound GTP, even in the presence of a GAP. Therefore, cancer cells comprise increased levels of active mutated Ras-GTP, which is thought to be a key factor for driving cancer cell proliferation.

Three main families of RAS-specific GEFs have been identified so far (reviewed in Vigil 2010 Nature Reviews Cancer; Rojas et al 2011, Genes & Cancer 2(3) 298-305). There are two son of sevenless proteins (SOS1 and SOS2), 4 different isoforms of Ras guanine nucleotide releasing proteins (Ras-GRP1-4) and two Ras guanine nucleotide releasing factors (Ras-GRF1 and 2). The SOS proteins are ubiquitously expressed and are recruited to sites of activated growth factors. Ras-GRFs are expressed mainly in the nervous system, where they are involved in Calcium-dependent activation of Ras. In contrast, Ras GRP proteins are expressed in hematopoietic cells and act in concert with non-receptor tyrosine kinases. In the context of cancer, mainly SOS proteins have been found to be involved.

Targeting Ras for cancer therapy has been a dream since the 1990s (Downward 2002 Nature Reviews Cancer, Krens et al. 2010 Drug Discovery Today). Due to the compact nature, the high affinity towards GDP and GTP in combination with high intracellular GTP concentrations, the Ras protein itself has always been considered to be undruggable, i.e. the chance to identify small chemical molecules that would bind to and inhibit active Ras was rated extremely low. Alternative approaches have been undertaken to reduce Ras signaling, e.g. by addressing more promising drug targets such as enzymes involved in the posttranslational modification of Ras proteins, especially farnesyltransferase and geranylgeranyltransferase (Berndt 2011 Nature Reviews Cancer). Inhibitors of farnesyltransferase (FTIs) were identified and developed with promising antitumor effects in preclinical models. Unexpectedly, in clinical trials these inhibitors have been of limited efficacy. Targeting upstream and downstream kinases involved in Ras signaling pathways has been more successful. Several drugs are and have been in clinical trials that inhibit different kinases, e.g. EGFR, Raf, MEK, Akt, PI3K (Takashima & Faller 2013 Expert Opin. Ther. Targets). Marketed cancer drugs are available that inhibit Raf, EGFR or MEK.

Nevertheless, there is still a large unmet need for the treatment of Ras-dependent tumors that are resistant against current therapies. Many research groups have been active to identify small molecules that target Ras directly (Ras small molecules have been reviewed in: Cox et al. 2014 Nature Reviews Drug Discovery, Spiegel et al. 2014 Nature Chemical Biology, Cromm 2015 Angewandte Chemie, Marin-Ramos et al Seminars in Cancer Biology). One group of inhibitors comprises small molecules that inhibit the interaction of Ras with its effectors Raf or PI3K. Another group of compounds acts as covalent inhibitors of a specific cysteine mutant form of K-Ras (glycine to cysteine point mutation G12C). The specific targeting of the Ras-G12C mutant might have the benefit of reduced side effects, as the wildtype Ras proteins should not be affected. Furthermore, several reports show small molecules and peptides that interrupt the GEF assisted activation of Ras (Hillig et al 2019 PNAS; Gray et al 2019 Angewandte Chemie). There seem to be several different binding sites possible that result in this mode of action. Inhibitors may bind to Ras or to the GEF in an allosteric or orthosteric fashion. All these approaches of direct Ras-targeting are in preclinical research stage. Stabilized peptides have been shown to be active in the nanomolar range. (Leshchiner et al. 2015 PNAS). Their usefulness as drugs in a clinical setting has to be awaited.

The Epidermal Growth Factor Receptor (EGFR) is a tyrosine kinase (TK) receptor that is activated upon binding to the Epidermal Growth Factor and other growth factor ligands, triggering several downstream pathways, including RAS/MAPK, PI3K/Akt and STAT that regulate different cellular processes, including DNA synthesis and proliferation (Russo A, Oncotarget. 4254, 2015). The family of HER (ErbB) receptor tyrosine kinases consists of four members, ie, epidermal growth factor receptors [EGFR (HER1 or ErbB1), HER2 (ErbB2, neu), HER3 (ErbB3), and HER4 (ErbB4)]. Overexpression, mutation, or aberrant activity of these receptors has been implicated in various types of cancer (Feldinger K, Breast Cancer (Dove Med Press), 2015, 7, 147).

First-Generation Inhibitors

Erlotinib and Gefitinib are small molecule inhibitors of the EGFR/HER-1 (human epidermal growth factor receptor) tyrosine kinase. Erlotinib and Gefitinib were developed as reversible and highly specific small-molecule tyrosine kinase inhibitors that competitively block the binding of adenosine triphosphate to its binding site in the tyrosine kinase domain of EGFR, thereby inhibiting autophosphorylation and blocking downstream signaling (Cataldo V D, N Engl J Med, 2011, 364, 947).

Second-Generation Inhibitors

Afatinib is an oral tyrosine kinase inhibitor (TKI) approved for the first-line treatment of patients with NSCLC whose tumors are driven by activating mutations of genes coding for epidermal growth factor receptor (EGFR). Afatinib is also an inhibitor of a specific EGFR mutation (T790M) that causes resistance to first-generation EGFR-targeted TKIs in about half of patients receiving those drugs. (Engle J A, Am J Health Syst Pharm 2014, 71 (22), 1933).

Neratinib, a pan-HER inhibitor, irreversible tyrosine kinase inhibitor binds and inhibits the tyrosine kinase activity of epidermal growth factor receptors, EGFR (or HER1), HER2 and HER4, which leads to reduced phosphorylation and activation of downstream signaling pathways. Neratinib has been shown to be effective against HER2-overexpressing or mutant tumors in vitro and in vivo. Neratinib is currently being investigated in various clinical trials in breast cancers and other solid tumors, including those with HER2 mutation (Feldinger K, Breast Cancer (Dove Med Press), 2015, 7, 147).

Dacomitinib is an irreversible inhibitor of EGFR, HER2, and HER4. In preclinical cell lines and xenograft studies, dacomitinib demonstrated activities against both activating EGFR mutations and EGFR T790M (Liao B C, Curr Opin Oncol. 2015, 27(2), 94).

Third-Generation Inhibitors

The third-generation EGFR-TKIs were designed to inhibit EGFR T790M while sparing wild-type EGFR.

AZD9291 (AstraZeneca, Macclesfield, UK), a mono-anilino-pyrimidine compound, is an irreversible mutant selective EGFR-TKI. This drug is structurally different from the first and second-generation EGFR-TKIs. In preclinical studies, it potently inhibited phosphorylation of EGFR in cell lines with activating EGFR mutations (EGFR del19 and EGFR L858R) and EGFR T790M. AZD9291 also caused profound and sustained tumor regression in tumor xenograft and transgenic mouse models harboring activating EGFR mutations and EGFR T790M. AZD9291 was less potent in inhibiting phosphorylation of wild-type EGFR cell lines (Liao B C, Curr Opin Oncol. 2015, 27(2), 94).

Rociletinib (CO-1686) (Clovis Oncology, Boulder, Colo), a 2,4-disubstituted pyrimidine molecule, is an irreversible mutant selective EGFR-TKI. In preclinical studies, CO-1686 led to tumor regression in cell-lines, xenograft models, and transgenic mouse models harboring activating EGFR mutations and EGFR T790M (Walter A O, Cancer Discov, 2013, 3(12), 1404).

HM61713 (Hanmi Pharmaceutical Company Ltd, Seoul, South Korea) is an orally administered, selective inhibitor for activating EGFR mutations and EGFR T790M. It has low activity against wild-type EGFR (Steuer C E, Cancer. 2015, 121(8), E1).

Hillig et al 2019 PNAS describe compounds like

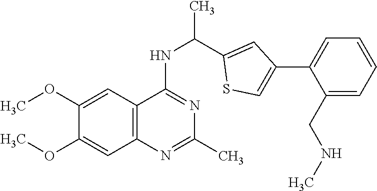 [SMILES: CNCc1ccccc1-c1csc(C(C)Nc2nc(C)nc3cc(OC)c(OC)cc23)c1]

as a potent SOS1 inhibitor and as a tool compound for further investigation of RAS-SOS1 biology in vitro.

FR 3 066 761 (Universite d'Orleans et al) describes compounds like

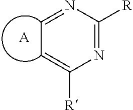

for the treatment of cancer.

WO 2018/134685 (Eisai Management Co. Ltd. et al) describes compounds like

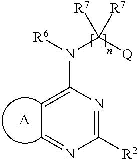

for the treatment and prevention of filarial worm infection.

WO 2018/172250 (Bayer Pharma A G) describes 2-methyl-quinazoline like

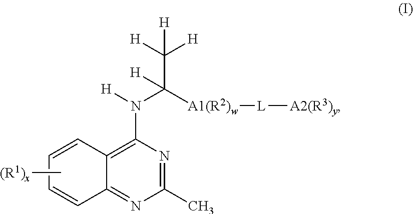

as inhibiting Ras-Sos interaction.

WO 2018/115380 (Boehringer Ingelheim) describes benzylamino substituted quinazolines like

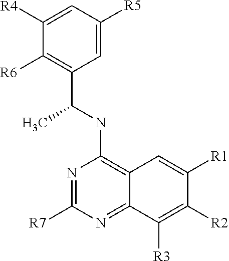

as SOS1 inhibitors.

WO 2019/122129 (Boehringer Ingelheim) describes benzylaminosubstituted pyridopyrimidinones like

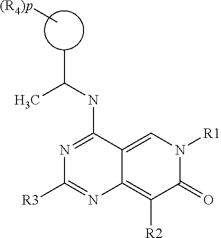

as SOS1 inhibitors.

WO 2020/180768 and WO 2020/180770 (Revolution) describes compounds of the following formulas:

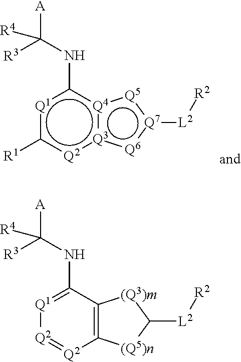

as SOS1 inhibitors.

It has now been found, and this constitutes the basis of the present invention, that the compounds of the present invention have surprising and advantageous properties.

In particular, the compounds of the present invention have surprisingly been found to effectively and selectively inhibit the Ras-Sos1 interaction and may therefore be used for the treatment or prophylaxis of hyper-proliferative disorders, in particular cancer.

### DESCRIPTION OF THE INVENTION

In accordance with a first aspect, the present invention covers compounds of general formula (I):

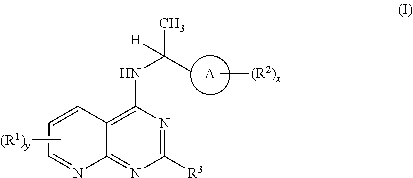

in which
A is selected from the group consisting of phenyl, naphthyl, heteroaryl and 9-10 membered bicyclic heterocyclyl;
R¹ is selected from
   —H; or
   -L-M, wherein
      L is selected from
         a single bond;
         —C(R^{a})(R^{b})—;
         —C(═O)—;
         —S(═O)₂—;
         —C(═O)—NR^{a}; or
         —S(═O)₂—NR^{a}—; and
         R^{a} and R_{b} independently can be —H;
         C₁₋₆-alkyl, optionally substituted with a halogen or —OH;
         C₃₋₈cycloalkyl, optionally substituted with a halogen or —OH; or
         or R^{a} and R_{b} together with the carbon atom they are attached to form a C₃₋₈-cycloalkyl or 4 to 6 membered heterocycloalkyl; and
      M is selected from
         C₁₋₆-alkyl; C₂₋₆-alkenyl; C₂₋₆-alkinyl; C₁₋₆-alkoxy; C₃₋₈-cycloalkyl; 4-6 membered heterocycloalkyl; phenyl; heteroaryl in which the C₁₋₆-alkyl; C₂₋₆-alkenyl; C₂₋₆-alkinyl; C₁₋₆-alkoxy; C₃₋₈-cycloalkyl; 4-6 membered heterocycloalkyl; phenyl and heteroaryl are all optionally substituted by one or more, identical or different Rₘselected from
         —OH, halogen, —CN; —C₁₋₆-alkyl; —C₃₋₆-cycloalkyl; —NRₙRₙ; —NRₙ—C(═O)—Rₙ; —NRₙ—S(═O)₂—Rₙ, —O—C₁₋₆-alkyl; —SRₙ; —S(O)—Rₙ, —S(O)₂—Rₙ or the bivalent oxo substituent, while the oxo substituent may only be a substituent in a non-aromatic ring and in which each Rₙ is identical or different and independently selected from C₁₋₆-alkyl or C₃₋₈-cycloalkyl;
         C₁₋₆-haloalkyl substituted with a 3 to 10 membered heterocyclyl; 3 to 10 membered heterocyclyl substituted with hydroxy, halogen, —NH₂, —SO₂—C₁₋₆-alkyl and the bivalent oxo-substituent, while the oxo-substituent may only be a substituent in a non-aromatic ring;
y is selected from 1 or 2;
R² is each independently selected from the group consisting of
   C₁₋₆-alkyl;
   C₁₋₆-haloalkyl;
   C₂₋₆-alkenyl;
   C₂₋₆-alkynyl;
   C₃₋₈-cycloalkyl;
   C₄₋₈-cycloalkenyl;
   3-10 membered heterocyclyl;
   phenyl and
   heteroaryl;
      wherein the C₁₋₆-alkyl, C₁₋₆-haloalkyl, C₂₋₆-alkenyl, C₂₋₆-alkynyl, C₃₋₈-cycloalkyl, C₄₋₈-cycloalkenyl, 3-10 membered heterocyclyl, phenyl and heteroaryl are all optionally substituted by one or more, identical or different R_{c} and/or R_{d};
         in which each R_{c} is independently selected from the group consisting of halogen, —CN, —C(═O)R_{d}, —C(═O)OR_{d}, —C(O)NR_{d}R_{d}, —NR_{d}R_{d}, —OR_{d}, —S(═O)₂—R_{d}, —S(═O)₂—NR_{d}R_{d}, —NH—C(═O)—R_{d}, —N(CH₃)—C(═O)—R_{d}, —N(C₁₋₆-alkyl)C(═O)—R_{d}, —NH—C(═O)OR_{d}, —N(CH₃)—C(═O)OR_{d}, —N(C₁₋₆-alkyl)-C(═O)OR_{d} and —NR_{d}—S(═O)₂—R_{d}; and
         in which each R_{d} is independently selected from the group consisting of hydrogen, C₁₋₆-alkyl, C₁₋₆-haloalkyl, C₂₋₆-alkenyl, C₂₋₆-alkynyl, C₃₋₈-cycloalkyl, C₄₋₈-cycloalkenyl, 3-10 membered heterocyclyl, phenyl and heteroaryl, wherein the C₁₋₆-alkyl, C₁₋₆-haloalkyl, C₂₋₆-alkenyl, C₂₋₆-alkynyl, C₃₋₈-cycloalkyl, C₄₋₈-cycloalkenyl, 3-10 membered heterocyclyl, phenyl and heteroaryl are all optionally substituted by one or more, identical or different Rₑ and/or R_{f};
         in which each Rₑ is independently selected from the group consisting of halogen, —CN, —C(═O)—R_{f}, —C(═O)OR_{f}, —C(═O) —NR_{f}R_{f}, —NR_{f}R_{f}, —OR_{f}, —S(═O)₂—R_{f}, —S(═O)₂NRR_{f}, —NHC(═O) R_{f}, —N(C₁₋₄ alkyl)C(═O) R_{f}, —NHC(═O)OR_{f} and —N(C₁₋₄ alkyl)C(═O)OR_{f}; and
         in which each R_{f} is independently selected from the group consisting of hydrogen, C₁₋₆-alkyl, C₁₋₆-haloalkyl, C₂₋₆-alkenyl, C₂₋₆-alkynyl, C₃₋₈-cycloalkyl, C₄₋₈-cycloalkenyl, 3-10 membered heterocyclyl, phenyl and heteroaryl;
x is selected from 1 or 2
R³ is selected from
   —H or —CH₃;
      or a stereoisomer, a tautomer, an N-oxide, a hydrate, a solvate, or a salt thereof, or a mixture of same.

### Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen or oxygen atom. Commonly, it is possible for the number of optional substituents, when present, to be 1, 2, 3, 4 or 5, in particular 1, 2 or 3.

As used herein, the term “one or more”, e.g. in the definition of the substituents of the compounds of general formula (I) of the present invention, means “1, 2, 3, 4 or 5, particularly 1, 2, 3 or 4, more particularly 1, 2 or 3, even more particularly 1 or 2”.

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one substituent.

As used herein, an oxo substituent represents an oxygen atom, which is bound to a carbon atom or to a sulfur atom via a double bond.

The term “ring substituent” means a substituent attached to an aromatic or nonaromatic ring which replaces an available hydrogen atom on the ring.

Should a composite substituent be composed of more than one part, e.g. (C₁-C₄-alkoxy)-(C₁-C₄-alkyl)-, it is possible for the position of a given part to be at any suitable position of said composite substituent, i.e. the C₁-C₄-alkoxy part can be attached to any carbon atom of the C₁-C₄-alkyl part of said (C₁-C₄-alkoxy)-(C₁-C₄-alkyl)-group. A hyphen at the beginning or at the end of such a composite substituent indicates the point of attachment of said composite substituent to the rest of the molecule. Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” or “halogen” means a fluorine, chlorine, bromine or iodine atom, particularly a fluorine, chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl, isobutyl, or tert-butyl group, more particularly 1, 2 or 3 carbon atoms (“C₁-C₃-alkyl”), e.g. a methyl, ethyl, n-propyl or isopropyl group.

The term “C₁-C₆-hydroxyalkyl” means a linear or branched, saturated, monovalent hydrocarbon group in which the term “C₁-C₆-alkyl” is defined supra, and in which 1, 2 or 3 hydrogen atoms are replaced with a hydroxy group, e.g. a hydroxymethyl, 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl group.

The term “C₁-C₆-alkylsulfanyl” means a linear or branched, saturated, monovalent group of formula (C₁-C₆-alkyl)-S—, in which the term “C₁-C₆-alkyl” is as defined supra, e.g. a methylsulfanyl, ethylsulfanyl, propylsulfanyl, isopropylsulfanyl, butylsulfanyl, sec-butylsulfanyl, isobutylsulfanyl, tert-butylsulfanyl, pentylsulfanyl, isopentylsulfanyl, hexylsulfanyl group.

The term “C₁-C₆-haloalkyl” means a linear or branched, saturated, monovalent hydrocarbon group in which the term “C₁-C₆-alkyl” is as defined supra, and in which one or more of the hydrogen atoms are replaced, identically or differently, with a halogen atom. Particularly, said halogen atom is a fluorine atom. Said C₁-C₆-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl.

The term “C₁-C₆-alkoxy” means a linear or branched, saturated, monovalent group of formula (C₁-C₆-alkyl)-O—, in which the term “C₁-C₆-alkyl” is as defined supra, e.g. a methoxy, ethoxy, n-propoxy, isopropoxy, n-butoxy, sec-butoxy, isobutoxy, tert-butoxy, pentyloxy, isopentyloxy or n-hexyloxy group, or an isomer thereof.

The term “C₁-C₆-haloalkoxy” means a linear or branched, saturated, monovalent C₁-C₆-alkoxy group, as defined supra, in which one or more of the hydrogen atoms is replaced, identically or differently, with a halogen atom. Particularly, said halogen atom is a fluorine atom. Said C₁-C₆-haloalkoxy group is, for example, fluoromethoxy, difluoromethoxy, trifluoromethoxy, 2,2,2-trifluoroethoxy or pentafluoroethoxy.

The term “C₂-C₆-alkenyl” means a linear or branched, monovalent hydrocarbon group, which contains one or two double bonds, and which has 2, 3, 4, 5 or 6 carbon atoms, particularly 2 or 3 carbon atoms (“C₂-C₃-alkenyl”), it being understood that in the case in which said alkenyl group contains more than one double bond, then it is possible for said double bonds to be isolated from, or conjugated with, each other. Said alkenyl group is, for example, an ethenyl (or “vinyl”), prop-2-en-1-yl (or “allyl”), prop-1-en-1-yl, but-3-enyl, but-2-enyl, but-1-enyl, pent-4-enyl, pent-3-enyl, pent-2-enyl, pent-1-enyl, hex-5-enyl, hex-4-enyl, hex-3-enyl, hex-2-enyl, hex-1-enyl, prop-1-en-2-yl (or “isopropenyl”), 2-methylprop-2-enyl, 1-methylprop-2-enyl, 2-methylprop-1-enyl, 1-methylprop-1-enyl, 3-methylbut-3-enyl, 2-methylbut-3-enyl, 1-methylbut-3-enyl, 3-methylbut-2-enyl, 2-methylbut-2-enyl, 1-methylbut-2-enyl, 3-methylbut-1-enyl, 2-methylbut-1-enyl, 1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, 3-methylpent-3-enyl, 2-methylpent-3-enyl, 1-methylpent-3-enyl, 4-methylpent-2-enyl, 3-methylpent-2-enyl, 2-methylpent-2-enyl, 1-methylpent-2-enyl, 4-methylpent-1-enyl, 3-methylpent-1-enyl, 2-methylpent-1-enyl, 1-methylpent-1-enyl, 3-ethylbut-3-enyl, 2-ethylbut-3-enyl, 1-ethylbut-3-enyl, 3-ethylbut-2-enyl, 2-ethylbut-2-enyl, 1-ethylbut-2-enyl, 3-ethylbut-1-enyl, 2-ethylbut-1-enyl, 1-ethylbut-1-enyl, 2-propylprop-2-enyl, 1-propylprop-2-enyl, 2-isopropylprop-2-enyl, 1-isopropylprop-2-enyl, 2-propylprop-1-enyl, 1-propylprop-1-enyl, 2-isopropylprop-1-enyl, 1-isopropylprop-1-enyl, 3,3-dimethylprop-1-enyl, 1-(1,1-dimethylethyl)ethenyl, buta-1,3-dienyl, penta-1,4-dienyl or hexa-1,5-dienyl group. Particularly, said group is vinyl or allyl.

The term “C₂-C₆-alkynyl” means a linear or branched, monovalent hydrocarbon group which contains one triple bond, and which contains 2, 3, 4, 5 or 6 carbon atoms, particularly 2 or 3 carbon atoms (“C₂-C₃-alkynyl”). Said C₂-C₆-alkynyl group is, for example, ethynyl, prop-1-ynyl, prop-2-ynyl (or “propargyl”), but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or 3,3-dimethylbut-1-ynyl group. Particularly, said alkynyl group is ethynyl, prop-1-ynyl or prop-2-ynyl.

The term “C₃-C₈-cycloalkyl” means a saturated, monovalent, mono- or bicyclic hydrocarbon ring which contains 3, 4, 5, 6, 7 or 8 carbon atoms (“C₃-C₈-cycloalkyl”). Said C₃-C₈-cycloalkyl group is for example, a monocyclic hydrocarbon ring, e.g. a cyclopropyl, cyclobutyl, cyclopentyl, cyclohexyl, cycloheptyl or cyclooctyl group, or a bicyclic hydrocarbon ring, e.g. a bicyclo[4.2.0]octyl or octahydropentalenyl.

The term “C₄-C₈-cycloalkenyl” means a monovalent, mono- or bicyclic hydrocarbon ring which contains 4, 5, 6, 7 or 8 carbon atoms and one double bond. Particularly, said ring contains 4, 5 or 6 carbon atoms (“C₄-C₆-cycloalkenyl”). Said C₄-C₈-cycloalkenyl group is for example, a monocyclic hydrocarbon ring, e.g. a cyclobutenyl, cyclopentenyl, cyclohexenyl, cycloheptenyl or cyclooctenyl group, or a bicyclic hydrocarbon ring, e.g. a bicyclo[2.2.1]hept-2-enyl or bicyclo[2.2.2]oct-2-enyl.

The term “C₃-C₈-cycloalkoxy” means a saturated, monovalent, mono- or bicyclic group of formula (C₃-C₈-cycloalkyl)-O—, which contains 3, 4, 5, 6, 7 or 8 carbon atoms, in which the term “C₃-C₈-cycloalkyl” is defined supra, e.g. a cyclopropyloxy, cyclobutyloxy, cyclopentyloxy, cyclohexyloxy, cycloheptyloxy or cyclooctyloxy group.

The term “spirocycloalkyl” means a saturated, monovalent bicyclic hydrocarbon group in which the two rings share one common ring carbon atom, and wherein said bicyclic hydrocarbon group contains 5, 6, 7, 8, 9, 10 or 11 carbon atoms, it being possible for said spirocycloalkyl group to be attached to the rest of the molecule via any one of the carbon atoms except the spiro carbon atom. Said spirocycloalkyl group is, for example, spiro[2.2]pentyl, spiro[2.3]hexyl, spiro[2.4]heptyl, spiro[2.5]octyl, spiro[2.6]nonyl, spiro[3.3]heptyl, spiro[3.4]octyl, spiro[3.5]nonyl, spiro[3.6]decyl, spiro[4.4]nonyl, spiro[4.5]decyl, spiro[4.6]undecyl or spiro[5.5]undecyl.

The terms “4- to 7-membered heterocycloalkyl” and “4- to 6-membered heterocycloalkyl” mean a monocyclic, saturated heterocycle with 4, 5, 6 or 7 or, respectively, 4, 5 or 6 ring atoms in total, which contains one or two identical or different ring heteroatoms from the series N, O and S, it being possible for said heterocycloalkyl group to be attached to the rest of the molecule via any one of the carbon atoms or, if present, a nitrogen atom.

Said heterocycloalkyl group, without being limited thereto, can be a 4-membered ring, such as azetidinyl, oxetanyl or thietanyl, for example; or a 5-membered ring, such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, for example, or a 7-membered ring, such as azepanyl, 1,4-diazepanyl or 1,4-oxazepanyl, for example.

Particularly, “4- to 6-membered heterocycloalkyl” means a 4- to 6-membered heterocycloalkyl as defined supra containing one ring nitrogen atom and optionally one further ring heteroatom from the series: N, O, S. More particularly, “5- or 6-membered heterocycloalkyl” means a monocyclic, saturated heterocycle with 5 or 6 ring atoms in total, containing one ring nitrogen atom and optionally one further ring heteroatom from the series: N, O.

The term “5- to 8-membered heterocycloalkenyl” means a monocyclic, unsaturated, non-aromatic heterocycle with 5, 6, 7 or 8 ring atoms in total, which contains one or two double bonds and one or two identical or different ring heteroatoms from the series: N, O, S; it being possible for said heterocycloalkenyl group to be attached to the rest of the molecule via any one of the carbon atoms or, if present, a nitrogen atom.

Said heterocycloalkenyl group is, for example, 4H-pyranyl, 2H-pyranyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl or 4H-[1,4]thiazinyl.

The term “heterospirocycloalkyl” means a bicyclic, saturated heterocycle with 6, 7, 8, 9, 10 or 11 ring atoms in total, in which the two rings share one common ring carbon atom, which “heterospirocycloalkyl” contains one or two identical or different ring heteroatoms from the series: N, O, S; it being possible for said heterospirocycloalkyl group to be attached to the rest of the molecule via any one of the carbon atoms, except the spiro carbon atom, or, if present, a nitrogen atom.

Said heterospirocycloalkyl group is, for example, azaspiro[2.3]hexyl, aza-spiro[3.3]heptyl, oxaazaspiro[3.3]heptyl, thiaazaspiro[3.3]heptyl, oxaspiro[3.3]heptyl, oxazaspiro[5.3]nonyl, oxazaspiro[4.3]octyl, azaspiro[4,5]decyl, oxazaspiro [5.5]undecyl, diazaspiro[3.3]heptyl, thiazaspiro[3.3]heptyl, thiazaspiro[4.3]octyl, azaspiro[5.5]undecyl, or one of the further homologous scaffolds such as spiro[3.4]-, spiro[4.4]-, spiro[2.4]-, spiro[2.5]-, spiro[2.6]-, spiro[3.5]-, spiro[3.6]-, spiro[4.5]- and spiro[4.6]-.

The term “fused heterocycloalkyl” means a bicyclic, saturated heterocycle with 6, 7, 8, 9 or 10 ring atoms in total, in which the two rings share two adjacent ring atoms, which “fused heterocycloalkyl” contains one or two identical or different ring heteroatoms from the series: N, O, S; it being possible for said fused heterocycloalkyl group to be attached to the rest of the molecule via any one of the carbon atoms or, if present, a nitrogen atom.

Said fused heterocycloalkyl group is, for example, azabicyclo[3.3.0]octyl, azabicyclo[4.3.0]nonyl, diazabicyclo[4.3.0]nonyl, oxazabicyclo[4.3.0]nonyl, thiazabicyclo[4.3.0]nonyl or azabicyclo[4.4.0]decyl.

The term “bridged heterocycloalkyl” means a bicyclic, saturated heterocycle with 7, 8, 9 or 10 ring atoms in total, in which the two rings share two common ring atoms which are not adjacent, which “bridged heterocycloalkyl” contains one or two identical or different ring heteroatoms from the series: N, O, S; it being possible for said bridged heterocycloalkyl group to be attached to the rest of the molecule via any one of the carbon atoms, except the spiro carbon atom, or, if present, a nitrogen atom.

Said bridged heterocycloalkyl group is, for example, azabicyclo[2.2.1]heptyl, oxazabicyclo[2.2.1]heptyl, thiazabicyclo[2.2.1]heptyl, diazabicyclo[2.2.1]heptyl, azabicyclo[2.2.2]octyl, diazabicyclo[2.2.2]octyl, oxazabicyclo[2.2.2]octyl, thiazabicyclo[2.2.2]octyl, azabicyclo[3.2.1]octyl, diazabicyclo[3.2.1]octyl, oxazabicyclo[3.2.1]octyl, thiazabicyclo[3.2.1]octyl, azabicyclo[3.3.1]nonyl, diazabicyclo[3.3.1]nonyl, oxazabicyclo[3.3.1]nonyl, thiazabicyclo[3.3.1]nonyl, azabicyclo[4.2.1]nonyl, diazabicyclo[4.2.1]nonyl, oxazabicyclo[4.2.1]nonyl, thiaza-bicyclo[4.2.1]nonyl, azabicyclo[3.3.2]decyl, diazabicyclo[3.3.2]decyl, oxazabicyclo[3.3.2]decyl, thiazabicyclo[3.3.2]decyl or azabicyclo[4.2.2]decyl.

The term “heteroaryl” means a monovalent, monocyclic, bicyclic or tricyclic aromatic ring having 5, 6, 8, 9, 10, 11, 12, 13 or 14 ring atoms (a “5- to 14-membered heteroaryl” group), particularly 5, 6, 9 or 10 ring atoms, which contains at least one ring heteroatom and optionally one, two or three further ring heteroatoms from the series: N, O and/or S, and which is bound via a ring carbon atom or optionally via a ring nitrogen atom (if allowed by valency).

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a tricyclic heteroaryl group, such as, for example, carbazolyl, acridinyl or phenazinyl; or a 9-membered heteroaryl group, such as, for example, benzofuranyl, benzothienyl, benzoxazolyl, benzisoxazolyl, benzimidazolyl, benzothiazolyl, benzotriazolyl, indazolyl, indolyl, isoindolyl, indolizinyl or purinyl; or a 10-membered heteroaryl group, such as, for example, quinolinyl, quinazolinyl, isoquinolinyl, cinnolinyl, phthalazinyl, quinoxalinyl or pteridinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

The term “C₁-C₆”, “C₁₋₆” as used in the present text, e.g. in the context of the definition of “C₁-C₆-alkyl”, “C₁-C₆-haloalkyl”, “C₁-C₆-hydroxyalkyl”, “C₁-C₆-alkoxy” or “C₁-C₆-haloalkoxy” means an alkyl group having a finite number of carbon atoms of 1 to 6, i.e. 1, 2, 3, 4, 5 or 6 carbon atoms.

Further, as used herein, the term “C₃-C₈” or “C₃₋₈”, as used in the present text, e.g. in the context of the definition of “C₃-C₈-cycloalkyl”, means a cycloalkyl group having a finite number of carbon atoms of 3 to 8, i.e. 3, 4, 5, 6, 7 or 8 carbon atoms.

When a range of values is given, said range encompasses each value and sub-range within said range.

For example:
“C₁-C₆” encompasses C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆;
“C₂-C₆” encompasses C₂, C₃, C₄, C₅, C₆, C₂-C₆, C₂-C₅, C₂-C₄, C₂-C₃, C₃- C₆, C₃-C₅, C₃-C₄, C₄-C₆, C₄-C₅, and C₅-C₆;
“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₃-C₈” encompasses C₃, C₄, C₅, C₆, C₇, C₈, C₃-C₈, C₃-C₇, C₃-C₆, C₃-C₅, C₃-C₄, C₄-C₈, C₄-C₇, C₄-C₆, C₄-C₅, C₅-C₈, C₅-C₇, C₅-C₆, C₆-C₈, C₆-C₇ and C₇-C₈;
“C₃-C₆” encompasses C₃, C₄, C₅, C₆, C₃-C₆, C₃-C₅, C₃-C₄, C₄-C₆, C₄-C₅, and C₅-C₆;
“C₄-C₈” encompasses C₄, C₅, C₆, C₇, C₈, C₄-C₈, C₄-C₇, C₄-C₆, C₄-C₅, C₅- C₈, C₅-C₇, C₅-C₆, C₆-C₈, C₆-C₇ and C₇-C₈;
“C₄-C₇” encompasses C₄, C₅, C₆, C₇, C₄-C₇, C₄-C₆, C₄-C₅, C₅-C₇, C₅-C₆ and C₆-C₇;
“C₄-C₆” encompasses C₄, C₅, C₆, C₄-C₆, C₄-C₅ and C₅-C₆;
“C₅-C₁₀” encompasses C₅, C₆, C₇, C₈, C₉, C₁₀, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉- C₁₀;
“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butylphenyl)sulfonyl]oxy and [(4-methoxyphenyl)sulfonyl]oxy.

The “heterocyclyl” denotes ring systems, which are derived from the previously defined cycloalkyl, cycloalkenyl and aryl by replacing one or more of the groups —CH₂— independently of one another in the hydrocarbon rings by the groups —O—, —S—, —NH—, —N(C₁₋₄-alkyl)- or by replacing one or more of the groups ═CH— by the group ═N—, wherein a total of not more than five heteroatoms may be present, at least one carbon atom must be present between two oxygen atoms and between two sulfur atoms or between an oxygen and a sulfur atom and the ring as a whole must have chemical stability. Heteroatoms may optionally be present in all the possible oxidation stages (sulfur→sulfoxide —SO—, sulfone —SO₂—; nitrogen→N-oxide). In a heterocyclyl there is no heteroaromatic ring, i.e. no heteroatom is part of an aromatic system. Examples of heterocyclyl are mentioned in WO 2019/122129 page 43, line 25 to page 47, line 5. Heterocyclic ring could be tetrahydrofuryl, pyrrolidinyl, pyrrolinyl, imidazolidinyl, thiazolidinyl, imidazolinyl, pyrazolidinyl, pyrazolinyl, piperidinyl, piperazinyl, oxiranyl, aziridinyl, azetidinyl 1,4-dioxanyl, azepanyl, diazepanyl, morpholinyl, thiomorpholinyl, homomorpholinyl, homopiperidinyl, homopiperazinyl, homothiomorpholinyl, thiomorpholinyl-S-oxide, thiomorpholinyl-S,S-dioxide, 1,3-dioxolanyl, tetrahydropyranyl, tetrahydrothiopyranyl, [1,4]-oxazepanyl, tetrahydrothienyl, homothiomorpholinyl-S,Sdioxide, oxazolidinonyl, dihydropyrazolyl, dihydropyrrolyl, dihydropyrazinyl, dihydropyridyl, dihydro-pyrimidinyl, dihydrofuryl, dihydropyranyl, tetrahydrothienyl-S-oxide, tetrahydrothienyl-S, S-dioxide, homothiomorpholinyl-S-oxide, 2,3-dihydroazet, 2H-pyrrolyl, 4H-pyranyl, 1,4-dihydropyridinyl, 8-aza-bicycle[3.2.1]octyl, 8-aza-bicyclo[5.1.0]octyl, 2-oxa-5-azabicyclo[2.2.1]heptyl, 8-oxa-3-aza-bicyclo[3.2.1]octyl, 3,8-diaza-bicyclo[3.2.1]octyl, 2,5-diaza-bicyclo[2.2.1]heptyl, 1-aza-bicyclo[2.2.2]octyl, s 3,8-diaza-bicyclo[3.2.1]octyl, 3,9-diaza-bicyclo[4.2.1]nonyl, 2,6-diaza-bicyclo[3.2.2]nonyl, 1,4-dioxa-spiro[4.5]decyl, 1-oxa-3,8-diaza-spiro[4.5]decyl, 2,6-diaza-spiro[3.3]heptyl, 2,7-diaza-spiro[4.4]nonyl, 2,6-diaza-spiro[3.4]octyl, 3,9-diaza-spiro[5.5]undecyl, 2.8-diazaspiro[4,5]decyl etc.

An “oxo substituent” in the context of the invention means an oxygen atom, which is bound to a carbon or a sulfur atom via a double bond. It is possible that two oxo substituents are bound to a sulfur atom.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C ¹⁵N, ¹⁷O, ¹⁸O, ³²P, ³³P ³³S, ³⁴S, ³⁵S, ³⁶S, ¹⁸F, ³⁶Cl, ⁸²Br, 123I, 124I, 125I, 129I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, MA, USA; and CombiPhos Catalysts, Inc., Princeton, NJ, USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

Preferred isomers are those which produce the more desirable biological activity. These separated, pure or partially purified isomers or racemic mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it is possible for the compounds of the present invention to exist as tautomers. For example, any compound of the present invention which contains an imidazopyridine moiety as a heteroaryl group for example can exist as a 1H tautomer, or a 3H tautomer, or even a mixture in any amount of the two tautomers, namely:

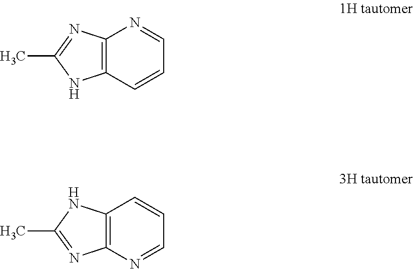

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates. Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

In accordance with a second embodiment of the first aspect, the present invention covers compounds of general formula (II)

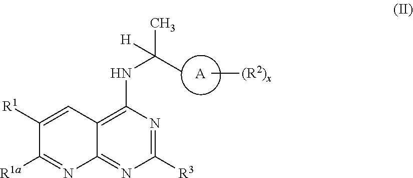

wherein
A is phenyl;
R¹ is selected from halogen, 5 to 10 membered mono or bicyclic heterocycloalkyl or heterocycloalkenyl with one or 2 nitrogen as heteroatoms and substituted by —CH₃, —C(═O)—CH₃ or —NH—C(═O)—CH₃,
R^{1a} is selected from hydrogen, —CH₃, CF₃ or —OCH₃;
R² is selected from hydrogen, halogen or C₁₋₆-alkyl optionally one or more time substituted by halogen and/or hydroxyl;
x is selected from 1 or 2 and
R³ is selected from hydrogen or —CH₃;
   or a stereoisomer, a tautomer, an N-oxide, a hydrate, a solvate, or a salt thereof, or a mixture of same.

In accordance with a third embodiment of the first aspect, the present invention covers compounds of general formula (II), supra, in which:
A is phenyl;
R¹ is selected from halogen

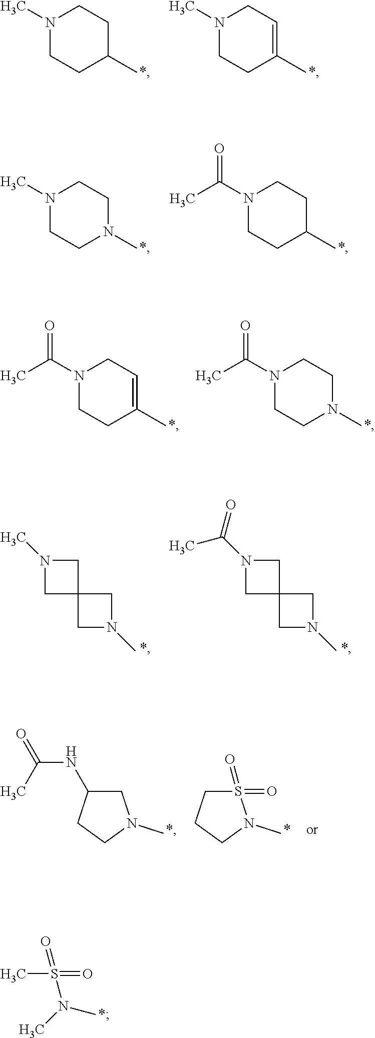

R^{1a} is selected from hydrogen, —CH₃, CF₃ or —OCH₃;
R² is each independently selected from —H, —CH₃, —F, —CF₃ or —CF₂—C(CH₃)₂—OH;
R³ is selected from hydrogen or —CH₃;
   or a stereoisomer, a tautomer, an N-oxide, a hydrate, a solvate, or a salt thereof, or a mixture of same.

In accordance with a fourth embodiment of the first aspect, the present invention covers compounds of general formula (III),

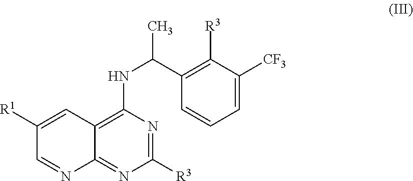

in which:
R¹ is selected from the group consisting of —Br,

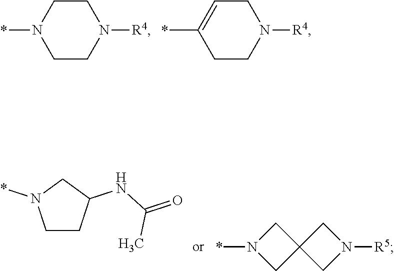

R³ is selected from the group consisting of —H and —CH₃;
R⁴ is selected from the group consisting of —CH₃ and —C(═O)—CH₃ and
R⁵ is selected from the group consisting of —C(═O)—CH₃ and —C(═O)OC(CH₃)₃
or a stereoisomer, a tautomer, an N-oxide, a hydrate, a solvate, or a salt thereof, or a mixture of same.

In accordance with another embodiment of the first aspect, the present invention covers compounds in which R^{1a} is —H or a stereoisomer, a tautomer, an N-oxide, a hydrate, a solvate, or a salt thereof, or a mixture of same.

In accordance with another embodiment of the first aspect, the present invention covers compounds in which R^{1a} is —CH₃ or a stereoisomer, a tautomer, an N-oxide, a hydrate, a solvate, or a salt thereof, or a mixture of same.

In accordance with another embodiment of the first aspect, the present invention covers compounds in which R^{1a} is —CF₃ or a stereoisomer, a tautomer, an N-oxide, a hydrate, a solvate, or a salt thereof, or a mixture of same.

In accordance with another embodiment of the first aspect, the present invention covers compounds in which R^{1a} is —O—CH₃ or a stereoisomer, a tautomer, an N-oxide, a hydrate, a solvate, or a salt thereof, or a mixture of same.

In accordance with another embodiment of the first aspect, the present invention covers compounds in which A is phenyl or a stereoisomer, a tautomer, an N-oxide, a hydrate, a solvate, or a salt thereof, or a mixture of same.

In accordance with another embodiment of the first aspect, the present invention covers compounds in which A is naphtyl and R² is H or a stereoisomer, a tautomer, an N-oxide, a hydrate, a solvate, or a salt thereof, or a mixture of same.

In accordance with another embodiment of the first aspect, the present invention covers compounds in which

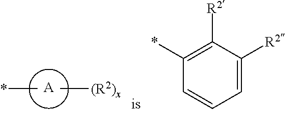

and in which R^{2′} is —CH₃ or a stereoisomer, a tautomer, an N-oxide, a hydrate, a solvate, or a salt thereof, or a mixture of same.

In accordance with another embodiment of the first aspect, the present invention covers compounds in which

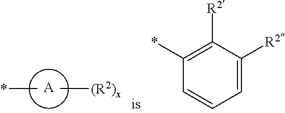

and in which R^{2′} is —F or a stereoisomer, a tautomer, an N-oxide, a hydrate, a solvate, or a salt thereof, or a mixture of same.
In accordance with another embodiment of the first aspect, the present invention covers compounds in which

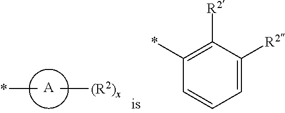

and in which R^{2′}is —H or a stereoisomer, a tautomer, an N-oxide, a hydrate, a solvate, or a salt thereof, or a mixture of same.

In accordance with another embodiment of the first aspect, the present invention covers compounds in which

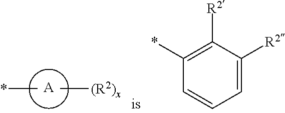

and in which R^{2″} is —CF₃ or a stereoisomer, a tautomer, an N-oxide, a hydrate, a solvate, or a salt thereof, or a mixture of same.

In accordance with another embodiment of the first aspect, the present invention covers compounds in which

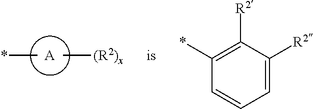

and in which R^{2′} is —CF₂H or a stereoisomer, a tautomer, an N-oxide, a hydrate, a solvate, or a salt thereof, or a mixture of same.

In accordance with another embodiment of the first aspect, the present invention covers compounds in which

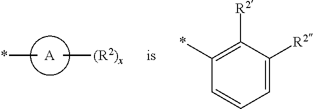

and in which R^{2″} is —CF₂—C(CH₃)₂—OH or a stereoisomer, a tautomer, an N-oxide, a hydrate, a solvate, or a salt thereof, or a mixture of same.

In accordance with another embodiment of the first aspect, the present invention covers compounds in which

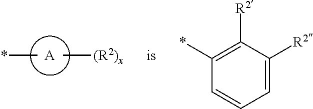

and in which R^{2″} is —CF₂—CH₂—OH or a stereoisomer, a tautomer, an N-oxide, a hydrate, a solvate, or a salt thereof, or a mixture of same.

In accordance with another embodiment of the first aspect, the present invention covers compounds in which R³ is H or a stereoisomer, a tautomer, an N-oxide, a hydrate, a solvate, or a salt thereof, or a mixture of same.

In accordance with another embodiment of the first aspect, the present invention covers compounds in which R³ is —CH₃ or a stereoisomer, a tautomer, an N-oxide, a hydrate, a solvate, or a salt thereof, or a mixture of same.

In a further embodiment of the first aspect, the present invention covers compounds of formula (I), (II) and/or (III) which are selected from the group consisting of:
6-bromo-N-{(1R)-1-[3-(trifluoromethyl)phenyl]ethyl}pyrido[2,3-d]pyrimidin-4-amine
N-{(3R)-1-[4-({(1R)-1-[3-(trifluoromethyl)phenyl]ethyl}amino)pyrido[2,3-d]pyrimidin-6-yl]pyrrolidin-3-yl}acetamide
6-(4-methylpiperazin-1-yl)—N-{(1R)-1-[3-(trifluoromethyl)phenyl]ethyl}pyrido[2,3-d]pyrimidin-4-amine
1-{4-[4-({(1R)-1-[3-(trifluoromethyl)phenyl]ethyl}amino)pyrido[2,3-d]pyrimidin-6-yl]piperazin-1-yl}ethan-1-one
1-{4-[4-({(1R)-1-[3-(trifluoromethyl)phenyl]ethyl}amino)pyrido[2,3-d]pyrimidin-6-yl]-3,6-dihydropyridin-1(2H)-yl}ethan-1-one
6-bromo-2-methyl-N-{(1R)-1-[2-methyl-3-(trifluoromethyl)phenyl]ethyl}pyrido[2,3-d]pyrimidin-4-amine tert-butyl 6-[2-methyl-4-({(1R)-1-[2-methyl-3-(trifluoromethyl)phenyl]ethyl}amino)pyrido[2,3-d]pyrimidin-6-yl]-2,6-diazaspiro[3.3]heptane-2-carboxylate
1-{6-[2-methyl-4-({(1R)-1-[2-methyl-3-(trifluoromethyl)phenyl]ethyl}amino)pyrido[2,3-d]pyrimidin-6-yl]-2,6-diazaspiro[3.3]heptan-2-yl}ethan-1-one
1-{4-[4-({(1R)-1-[3-(trifluoromethyl)phenyl]ethyl}amino)pyrido[2,3-d]pyrimidin-6-yl]piperidin-1-yl}ethan-1-one
1-{4-[4-({(1R)-1-[2-methyl-3-(trifluoromethyl)phenyl]ethyl}amino)pyrido[2,3-d]pyrimidin-6-yl]piperazin-1-yl}ethan-1-one
N-{(3R)-1-[4-({(1R)-1-[2-methyl-3-(trifluoromethyl)phenyl]ethyl}amino)pyrido[2,3-d]pyrimidin-6-yl]pyrrolidin-3-yl}acetamide
1-{4-[4-({(1R)-1-[2-methyl-3-(trifluoromethyl)phenyl]ethyl}amino)pyrido[2,3-d]pyrimidin-6-yl]-3,6-dihydropyridin-1(2H)-yl}ethan-1-one
1-{6-[2-methyl-4-({(1R)-1-[2-methyl-3-(trifluoromethyl)phenyl]ethyl}amino)pyrido[2,3-d]pyrimidin-6-yl]-2,6-diazaspiro[3.3]heptan-2-yl}ethan-1-one-hydrogen chloride (1/1) 1-{(1 S,4S)-5-[2-methyl-4-({(1R)-1-[2-methyl-3-(trifluoromethyl)phenyl]ethyl}amino)pyrido[2,3-d]pyrimidin-6-yl]-2,5-diazabicyclo[2.2.1]heptan-2-yl}ethan-1-one
2-methyl-6-(4-methylpiperazin-1-yl)—N-{(1R)-1-[2-methyl-3-(trifluoromethyl)phenyl]ethyl}pyrido[2,3-d]pyrimidin-4-amine
N-{(3R)-1-[2-methyl-4-({(1R)-1-[2-methyl-3-(trifluoromethyl)phenyl]ethyl}amino)pyrido[2,3-d]pyrimidin-6-yl]pyrrolidin-3-yl}acetamide
1-{4-[2-methyl-4-({(1R)-1-[2-methyl-3-(trifluoromethyl)phenyl]ethyl}amino)pyrido[2,3-d]pyrimidine-6-sulfonyl]piperazin-1-yl}ethan-1-one
N-{(3R)-1-[2,7-dimethyl-4-({(1R)-1-[2-methyl-3-(trifluoromethyl)phenyl]ethyl}amino)pyrido[2,3-d]pyrimidin-6-yl]pyrrolidin-3-yl}acetamide
1-{4-[2,7-dimethyl-4-({(1R)-1-[2-methyl-3-(trifluoromethyl)phenyl]ethyl}amino)pyrido[2,3-d]pyrimidin-6-yl]piperazin-1-yl}ethan-1-one
1-{6-[7-methoxy-2-methyl-4-({(1R)-1-[2-methyl-3-(trifluoromethyl)phenyl]ethyl}amino)pyrido[2,3-d]pyrimidin-6-yl]-2,6-diazaspiro[3.3]heptan-2-yl}ethan-1-one
1-{4-[7-methoxy-2-methyl-4-({(1R)-1-[2-methyl-3-(trifluoromethyl)phenyl]ethyl}amino)pyrido[2,3-d]pyrimidin-6-yl]-3,6-dihydropyridin-1(2H)-yl}ethan-1-one
1-{4-[7-methoxy-2-methyl-4-({(1R)-1-[2-methyl-3-(trifluoromethyl)phenyl]ethyl}amino)pyrido[2,3-d]pyrimidin-6-yl]piperidin-1-yl}ethan-1-one
2-[7-methoxy-2-methyl-4-({(1R)-1-[2-methyl-3-(trifluoromethyl)phenyl]ethyl}amino)pyrido[2,3-d]pyrimidin-6-yl]-1lambda⁶,2-thiazolidine-1,1-dione
1-{6-[4-({(1R)-1-[3-(1,1-difluoro-2-hydroxyethyl)-2-fluorophenyl]ethyl}amino)-2-methyl-7-(trifluoromethyl)pyrido[2,3-d]pyrimidin-6-yl]-2,6-diazaspiro[3.3]heptan-2-yl}ethan-1-one
1-{4-[2-methyl-4-({(1R)-1-[2-methyl-3-(trifluoromethyl)phenyl]ethyl}amino)pyrido[2,3-d]pyrimidin-6-yl]-3,6-dihydropyridin-1(2H)-yl}ethan-1-one
1-{4-[2-methyl-4-({(1R)-1-[2-methyl-3-(trifluoromethyl)phenyl]ethyl}amino)pyrido[2,3-d]pyrimidin-6-yl]-3,6-dihydropyridin-1(2H)-yl}ethan-1-one hydrogen chloride (1/1)
1-{7-[2-methyl-4-({(1R)-1-[2-methyl-3-(trifluoromethyl)phenyl]ethyl}amino)pyrido[2,3-d]pyrimidin-6-yl]-2-azaspiro[3.5]non-6-en-2-yl}ethan-1-one
1-{7-[2-methyl-4-({(1R)-1-[2-methyl-3-(trifluoromethyl)phenyl]ethyl}amino)pyrido[2,3-d]pyrimidin-6-yl]-2-azaspiro[3.5]nonan-2-yl}ethan-1-one
1-{6-[4-({(1R)-1-[3-(difluoromethyl)-2-fluorophenyl]ethyl}amino)-2-methylpyrido[2,3-d]pyrimidin-6-yl]-2,6-diazaspiro[3.3]heptan-2-yl}ethan-1-one
1-{3-[2-methyl-4-({(1R)-1-[2-methyl-3-(trifluoromethyl)phenyl]ethyl}amino)pyrido[2,3-d]pyrimidin-6-yl]-2,5-dihydro-1H-pyrrol-1-yl}ethan-1-one
1-{(3RS)-3-[2-methyl-4-({(1R)-1-[2-methyl-3-(trifluoromethyl)phenyl]ethyl}amino)pyrido[2,3-d]pyrimidin-6-yl]pyrrolidin-1-yl}ethan-1-one
6-methoxy-2-methyl-N-{(1R)-1-[2-methyl-3-(trifluoromethyl)phenyl]ethyl}pyrido[2,3-d]pyrimidin-4-amine
N-methyl-N-[2-methyl-4-({(1R)-1-[2-methyl-3-(trifluoromethyl)phenyl]ethyl}amino)pyrido[2,3-d]pyrimidin-6-yl]methanesulfonamide
2-[2-methyl-4-({(1R)-1-[2-methyl-3-(trifluoromethyl)phenyl]ethyl}amino)pyrido[2,3-d]pyrimidin-6-yl]-1lambda⁶,2-thiazolidine-1,1-dione
1-{4-[2-methyl-4-({(1R)-1-[2-methyl-3-(trifluoromethyl)phenyl]ethyl}amino)pyrido[2,3-d]pyrimidin-6-yl]piperazin-1-yl}ethan-1-one
1-{4-[2-methyl-4-({(1R)-1-[2-methyl-3-(trifluoromethyl)phenyl]ethyl}amino)pyrido[2,3-d]pyrimidin-6-yl]-4-oxo-4lambda⁵-piperazin-1-yl}ethan-1-one
1-{6-[4-({(1R)-1-[3-(difluoromethyl)-2-fluorophenyl]ethyl}amino)-2,7-dimethylpyrido[2,3-d]pyrimidin-6-yl]-2,6-diazaspiro[3.3]heptan-2-yl}ethan-1-one
6-methoxy-2,7-dimethyl-N-{(1R)-1-[2-methyl-3-(trifluoromethyl)phenyl]ethyl}pyrido[2,3-d]pyrimidin-4-amine
2-[4-({(1R)-1-[3-(difluoromethyl)-2-fluorophenyl]ethyl}amino)-2,7-dimethylpyrido[2,3-d]pyrimidin-6-yl]-1lambda⁶,2-thiazolidine-1,1-dione
1-{3-[2-methyl-4-({(1R)-1-[2-methyl-3-(trifluoromethyl)phenyl]ethyl}amino)pyrido[2,3-d]pyrimidin-6-yl]azetidin-1-yl}ethan-1-one
1-{4-[2-methyl-4-({(1R)-1-[2-methyl-3-(trifluoromethyl)phenyl]ethyl}amino)-7-(trifluoromethyl)pyrido[2,3-d]pyrimidin-6-yl]-3,6-dihydropyridin-1 (2H)-yl}ethan-1-one
1-{4-[2-methyl-4-({(1R)-1-[2-methyl-3-(trifluoromethyl)phenyl]ethyl}amino)-7-(trifluoromethyl)pyrido[2,3-d]pyrimidin-6-yl]piperidin-1-yl}ethan-1-one
1-{4-[4-({(1R)-1-[3-(difluoromethyl)-2-fluorophenyl]ethyl}amino)-2-methylpyrido[2,3-d]pyrimidin-6-yl]-3,6-dihydropyridin-1(2H)-yl}ethan-1-one
1-{4-[4-({(1R)-1-[3-(difluoromethyl)-2-fluorophenyl]ethyl}amino)-2-methylpyrido[2,3-d]pyrimidin-6-yl]piperidin-1-yl}ethan-1-one
2-methyl-N-{(1R)-1-[2-methyl-3-(trifluoromethyl)phenyl]ethyl}-6-(pyrimidin-5-yl)pyrido[2,3-d]pyrimidin-4-amine
1-{4-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-3,6-dihydropyridin-1 (2H)-yl}ethan-1-one
1-{4-[4-({(1R)-1-[3-(1,1-difluoro-2-hydroxy-2-methylpropyl)-2-fluorophenyl]ethyl}amino)-2-methylpyrido[2,3-d]pyrimidin-6-yl]piperidin-1-yl}ethan-1-one
6-[2-methyl-4-({(1R)-1-[2-methyl-3-(trifluoromethyl)phenyl]ethyl}amino)pyrido[2,3-d]pyrimidin-6-yl]-2,6-diazaspiro[3.3]heptane-2-carboxamide
1-{3-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-3,6-diazabicyclo[3.1.1]heptan-6-yl}ethan-1-one
1-{(1 S,4S)-5-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-2,5-diazabicyclo[2.2.1]heptan-2-yl}ethan-1-one
1-{(1 R,4R)-5-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-2,5-diazabicyclo[2.2.1]heptan-2-yl}ethan-1-one
1,1-difluoro-1-{2-fluoro-3-[(1R)-1-{[2-methyl-6-(4-methylpiperazin-1-yl)pyrido[2,3-d]pyrimidin-4-yl]amino}ethyl]phenyl}-2-methylpropan-2-ol
1-{4-[4-({(1R)-1-[3-(1,1-difluoro-2-hydroxy-2-methylpropyl)-2-fluorophenyl]ethyl}amino)-2-methylpyrido[2,3-d]pyrimidin-6-yl]piperazin-1-yl}ethan-1-one
1-{6-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-2,6-diazaspiro[3.3]heptan-2-yl}ethan-1-one
N-{(3R)-1-[4-({(1R)-1-[3-(1,1-difluoro-2-hydroxy-2-methylpropyl)-2-fluorophenyl]ethyl}amino)-2-methylpyrido[2,3-d]pyrimidin-6-yl]pyrrolidin-3-yl}acetamide
1-{4-[4-({(1R)-1-[3-(difluoromethyl)-2-fluorophenyl]ethyl}amino)-2,7-dimethylpyrido[2,3-d]pyrimidin-6-yl]-3,6-dihydropyridin-1 (2H)-yl}ethan-1-one
1-{4-[4-({(1R)-1-[3-(difluoromethyl)-2-fluorophenyl]ethyl}amino)-2,7-dimethylpyrido[2,3-d]pyrimidin-6-yl]piperidin-1-yl}ethan-1-one
2-[2,7-dimethyl-4-({(1R)-1-[2-methyl-3-(trifluoromethyl)phenyl]ethyl}amino)pyrido[2,3-d]pyrimidin-6-yl]-1lambda⁶,2-thiazolidine-1,1-dione
2-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]-1lambda⁶,2-thiazolidine-1,1-dione
1-{6-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]-2,6-diazaspiro[3.3]heptan-2-yl}ethan-1-one
4-acetyl-1-[2-methyl-4-({(1R)-1-[2-methyl-3-(trifluoromethyl)phenyl]ethyl}amino)pyrido[2,3-d]pyrimidin-6-yl]piperazin-2-one
1-{4-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]-5-methyl-3,6-dihydropyridin-1(2H)-yl}ethan-1-one
1-{6-[2,7-dimethyl-4-({(1R)-1-[2-methyl-3-(trifluoromethyl)phenyl]ethyl}amino)pyrido[2,3-d]pyrimidin-6-yl]-2,6-diazaspiro[3.3]heptan-2-yl}ethan-1-one
1-{(1 S,4S)-5-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]-2,5-diazabicyclo[2.2.1]heptan-2-yl}ethan-1-one
1-{4-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]-3,6-dihydropyridin-1 (2H)-yl}ethan-1-one
1-{4-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]piperidin-1-yl}ethan-1-one
1-{4-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]piperazin-1-yl}ethan-1-one
4-acetyl-1-[2,7-dimethyl-4-({(1R)-1-[2-methyl-3-(trifluoromethyl)phenyl]ethyl}amino)pyrido[2,3-d]pyrimidin-6-yl]piperazin-2-one
1-{6-[4-({(1R)-1-[3-(difluoromethyl)-2-fluorophenyl]ethyl}amino)-2-methyl-7-(trifluoromethyl)pyrido[2,3-d]pyrimidin-6-yl]-2,6-diazaspiro[3.3]heptan-2-yl}ethan-1-one
1-{6-[4-({(1R)-1-[3-(1,1-difluoro-2-hydroxy-2-methylpropyl)-2-fluorophenyl]ethyl}amino)-2-methyl-7-(trifluoromethyl)pyrido[2,3-d]pyrimidin-6-yl]-2,6-diazaspiro[3.3]heptan-2-yl}ethan-1-one
1-{6-[2-methyl-4-({(1R)-1-[2-methyl-3-(trifluoromethyl)phenyl]ethyl}amino)-7-(trifluoromethyl)pyrido[2,3-d]pyrimidin-6-yl]-2,6-diazaspiro[3.3]heptan-2-yl}ethan-1-one
   or a stereoisomer, a tautomer, an N-oxide, a hydrate, a solvate, or a salt thereof, or a mixture of same.

In a particular further embodiment of the first aspect, the present invention covers combinations of two or more of the above mentioned embodiments under the heading “further embodiments of the first aspect of the present invention”.

The present invention covers any sub-combination within any embodiment or aspect of the present invention of compounds of general formula (I), supra.

The present invention covers any sub-combination within any embodiment or aspect of the present invention of intermediate compounds of general formula (I).

The present invention covers the compounds of general formula (I) which are disclosed in the Example Section of this text, infra.

The compounds according to the invention of general formula (I) can be prepared according to the following schemes 1, 2, 3, 4, 5 and 6. The schemes and procedures described below illustrate synthetic routes to the compounds of general formula (I) of the invention and are not intended to be limiting. It is clear to the person skilled in the art that the order of transformations as exemplified in schemes 1, 2, 3, 4, 5 and 6 can be modified in various ways. The order of transformations exemplified in these schemes is therefore not intended to be limiting. In addition, interconversion of any of the substituents, R¹, R^{1a}, R², R³, R⁴, R^{a} and R^{b} can be achieved before and/or after the exemplified transformations. These transformations can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 4^{th} edition, Wiley 2006). Specific examples are described in the subsequent paragraphs.

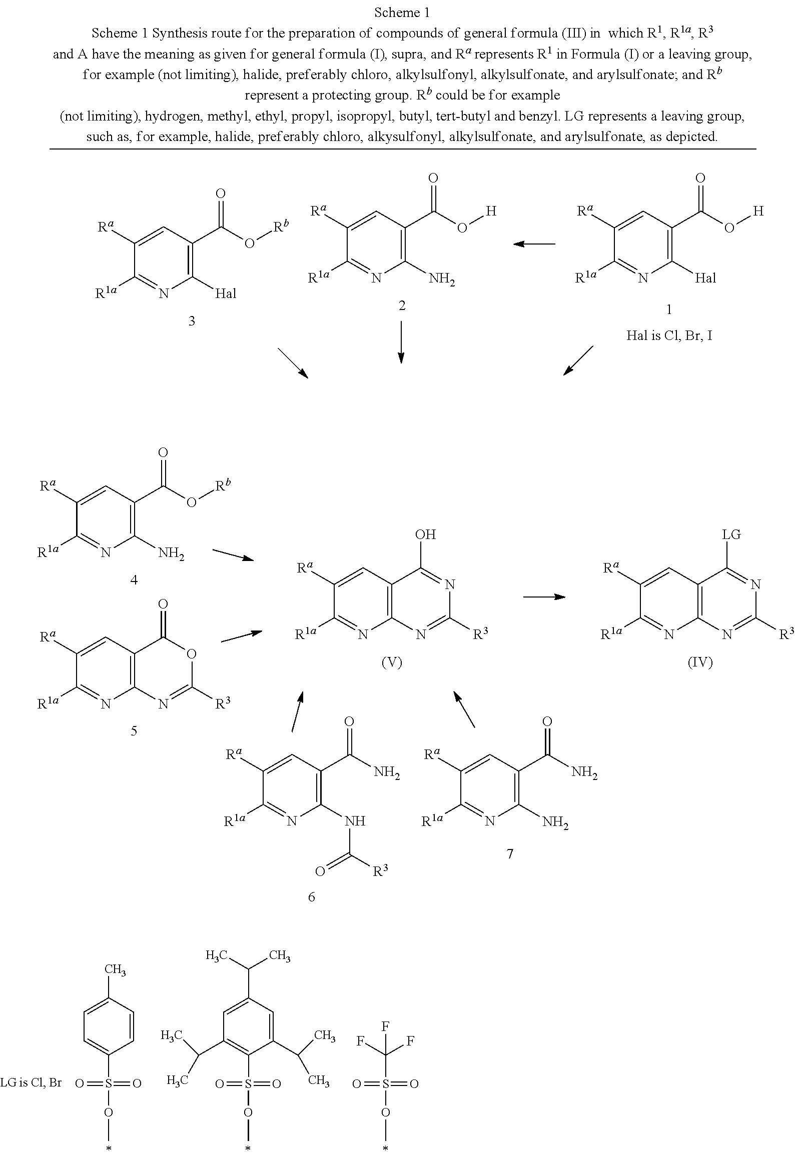

### Step 1->General Formula (IV) (Scheme 1)

Bicyclic pyrimidine formation: Alternatively, halogen substituted benzoic acid derivative of general formula 1 (which could be commercially available or described in the literature) could be converted to the corresponding bicyclic pyrimidine formation IV in analogy to literature procedures used to synthesize azaquinolines. Typically, derivative 1 is reacted with ammonia to form a derivative of general formula 2, preferably under elevated temperatures, optionally under high pressure, in water or an organic solvent or mixture thereof, such as for example, 1,2-dichloroethane, THF, methanol, ethanol. For example, see WO2017069275, US20030199511 and US20030187026 and the references therein. Alternatively, derivative 1 can be converted to the corresponding acid chloride, with for example thionyl chloride, oxalyl chloride, in an organic solvent, optionally with a drop of DMF, optionally at elevated temperature, in an organic solvent. The corresponding acid chloride can be treated with an imidamide or a salt thereof, with an inorganic base such as for example, caesium carbonate, sodium carbonate, potassium carbonate, or an organic base such as for example triethylamine, diisopropylethylamine or pyridine with or without DMAP, optionally using metal-catalyzed reactions, optionally in the presence of a ligand, in an organic solvent such as for example DMF, toluene, 1,4-dioxane/water at elevated temperature. For example, see WO2007134986, Bioorg. Med. Chem. Lett., 2015, 23, 3013 and the references therein.

### Step 2->General Formula (IV) (Scheme 1)

Bicyclic pyrimidine formation: Alternatively, amino substituted benzoic acid derivative of general formula 2 (which could be commercially available or described in the literature) could be converted to the corresponding bicyclic pyrimidine formation IV in analogy to literature procedures used to synthesize azaquinolines. Typically, derivative 2 is reacted with acetamidine or an imidamide, optionally with a base such as for example potassium carbonate or sodium hydroxide or triethylamine, diisopropylethylamine, 1,8-diazabicyclo[5.4.0]undec-7-ene or pyridine in an organic solvent such as for example DMF at elevated temperature. For example, see WO2004071460, WO2015155306 and. Chem. Med. Chem., 2014, 9, 2516.

### Step 3->General Formula (IV) (Scheme 1)

Bicyclic pyrimidine formation: Alternatively, halogen substituted benzoic ester derivative of general formula 3 (which could be commercially available or described in the literature) could be converted to the corresponding bicyclic pyrimidine formation (IV) in analogy to literature procedures used to synthesize azaquinolines. Typically, derivative 3 could be reacted with an imidamide or a salt there of, an inorganic base such as for example, caesium carbonate, sodium carbonate, potassium carbonate, or a organic base such as for example, triethylamine, diisopropylethylamine, 1,8-diazabicyclo[5.4.0]undec-7-ene or pyridine with or without DMAP, optionally using a metal-catalyzed reactions, optional in the presence of a ligand, in an organic solvent such as for example DMF, toluene, 1,4-dioxane/water at elevated temperature. For example, see Chem. Commun., 2008, 47, 6333; Bioorg. Med. Chem. Lett., 2013, 23, 3325; WO2018118735, WO2007134986 and references therein.

### Step 4->General Formula (IV) (Scheme 1)

Bicyclic pyrimidine formation: Alternatively, amino substituted benzoic ester derivative of general formula 4 (which could be commercially available or described in the literature) could be converted to the corresponding bicyclic pyrimidine formation (IV) in analogy to literature procedures used to synthesize azaquinolines. Typically, derivative 3 could be reacted with a nitrile, carboxylic acid chloride, carboxylic acid anhydride, imidamide or a salt there of, in the presence of an acid or a base, in water or an organic solvent, or mixtures thereof, such as for example DMF, toluene, 1,4-dioxane/water at elevated temperature. For example, see J. Med. Chem., 2018, 61, 3389; J. Med. Chem., 2019, 62, 9772; WO2004071460, WO2007134986 and references therein.

### Step 5->General Formula (IV) (Scheme 1)

Bicyclic pyrimidine formation: Alternatively, benzoxazinone derivative of general formula 5 (which could be commercially available or could be prepared in analogy to literature procedures) could be converted to the corresponding bicyclic pyrimidine formation (IV) in analogy to literature procedures used to synthesize azaquinolines. Typically, derivative 4 could be reacted with ammonium acetate in an organic solvent at elevated temperature. For example, see J. Med. Chem., 2019, 62, 9772; J. Med. Chem., 2011, 54, 6734; Bioorg. Med. Chem., 2014, 22, 5487 or WO2005105760 and references therein.

### Step 6->General Formula (IV) (Scheme 1)

Bicyclic pyrimidine formation: Alternatively, benzoic acid amide derivative of general formula 6 (which could be commercially available or described in the literature) could be converted to the corresponding bicyclic pyrimidine formation (IV) in analogy to literature procedures used to synthesize azaquinolines. Typically, derivative 6 could be reacted with a base such as for example sodium hydroxide in a solvent such as for example water at elevated temperature. For example, see Monatshefte Für Chemie, 1987, 118, 399; WO2007134986, WO2013016999; WO2012028578 and references therein.

### Step 7->General Formula (IV) (Scheme 1)

Bicyclic pyrimidine formation: Alternatively, amino benzoic acid amide derivative of general formula 6 (which could be commercially available or described in the literature) could be converted to the corresponding bicyclic pyrimidine formation (IV) in analogy to literature procedures used to synthesize azaquinolines. Typically, derivative 7 could be reacted with an organic acid at elevated temperature, an organic acid amide or carboxylic acid anhydrides or using copper-catalyzed reactions, optionally with a base, water or an organic solvent or mixtures thereof, preferably at elevated temperatures. For example, see Eur. J. Org. Chem., 2020, 2730; Polish Journal of Pharmacology and Pharmacy, 1985, 37, 541; Heterocycles, 2015, 90, 857; Yakugaku Zasshi, 1977, 97, 1022 and references therein.

For LG=chloro or bromo typically with phosphorus oxytrichloride or phosphorus oxytribromide, respectively, with or without N,N-dimethylaniline or N,N-diisopropylethylamine with or without an organic solvent such as for example toluene at elevated temperatures is used. For examples, see US2012/53174; WO2012/30912 or WO2012/66122 and references therein.

For LG=2,4,6-triisopropylsulfonate typically 2,4,6-triisopropylbenzenesulfonyl chloride, a base such as for example triethylamine and/or DMAP in an organic solvent such as for example dichloromethane is used. For examples see WO2010/99379 US2012/53176 and references therein.

For LG=tosylate typically 4-methylbenzene-1-sulfonyl chloride, a base such as for example triethylamine or potassium carbonate and/or DMAP in an organic solvent such as for example dichloromethane or acetonitrile is used. For examples see Organic Letters, 2011, 4374 or Bioorg. Med. Chem. Lett., 2013, 2663 and references therein. For LG=trifluoromethanesulfonate typically N,N-bis(trifluoromethylsulfonyl)aniline or trifluoromethanesulfonic anhydride, a base such as for example triethylamine or 1,8-diazabicyclo[5.4.0]undec-7-ene and/or DMAP in an organic solvent such as for example dichloromethane is used. For examples see J. Am. Chem. Soc., 2015, 13433 or WO2014/100501 and references therein.

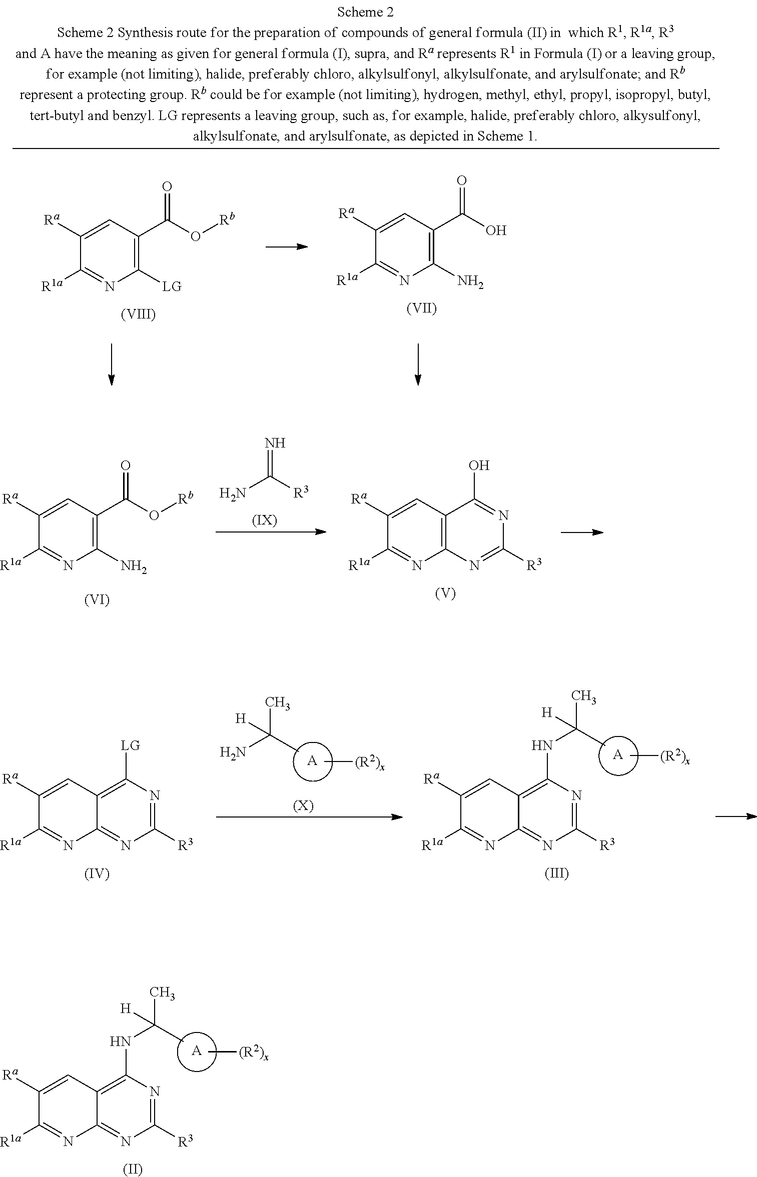

Compounds of general formula (VIII) are well-known in the public domain, commercially available or could be synthesized by known synthetic routes and are known to those skilled in the art.

Compounds of general formula (VII) are well-known in the public domain, commercially available or could be synthesized from compounds of general formula (VIII) by known synthetic routes, for example, acidic or basic ester hydrolysis.

Compounds of general formula (VI) are well-known in the public domain, commercially available or could be synthesized by known synthetic routes from compounds of general formula (VIII), for example, via formation of the nucleophilic substitution reaction (S_{N}Ar) with an nitrogen-containing nucleophile, see the teachings of see WO2017069275, US20030199511 and US20030187026.

The conversion of compounds of general formula (VI) to compounds of general formula (V) are well-documented in the public domain, for such transformations see the teachings of J. Med. Chem., 2018, 61, 3389; J. Med. Chem., 2019, 62, 9772; WO2004071460 and WO2007134986. Compounds of general formula (IV) can also be found available at commercial sources.

Alternatively, compounds of general formula (V) can be formed from compounds of general formula (VII) and are well-documented in the public domain, see the teachings of WO2004071460, WO2015155306 and. Chem. Med. Chem., 2014, 9, 2516.

Additional compounds of general formula (IV) can be formed from compounds of general formula (V), with compounds of general formula (X) using dehydrative conjugation methods. Such methods are known using coupling reagents like benzotriazol-1-yloxytris(dimethylamino)phosphonium hexafluorophosphate (BOP) and benzotriazol-1-yl-oxytripyrrolidinophosphonium hexafluorophosphate (PyBOP), and the teachings of J. Org. Chem., 2007, 72, 10194; Advanced Synthesis & Catalysis, 2018, 360, 4764; Bioorg. Med. Chem., 2019, 27, 931; WO 2011028741 A1; are in the public domain.

Alternatively, compounds of general formula (III) can be formed in a two-step process, whereby compounds of general formula (V) are converted to compounds of general formula (IV) using standard well-documented methods, such as, when LG=Cl using phosphorus oxytrichloride, or LG=Br using phosphorus oxytribromide, or LG=tosylate typically 4-methylbenzene-1-sulfonyl chloride, a base such as for example triethylamine or potassium carbonate and/or DMAP in an organic solvent such as for example dichloromethane or acetonitrile is used. For examples see Organic Letters, 2011, 4374 or Bioorg. Med. Chem. Lett., 2013, 2663 and references therein

The subsequently the compounds of general formula (IV) can be converted to compounds of general formula (III), using a nucleophilic substitution reaction (S_{N}Ar) with compounds of general formula (X) which are well-documented in the public domain and are known to those skilled in the art.

Compounds of general formula (III) can be converted to compounds of general formula (II), whereby R^{a} is a functional group that could be modified further. Such transformations are wide-ranging and are known to those skilled in the art. For example (not-limiting) when R^{a} is a leaving group, such as, for example, halide, alkylsulfonyl, metal catalysed reactions could be carried out, such as, for example, as Suzuki, Sonogashiri, Buchwald-Hartwig, Heck, Stille, Ullman reactions. In addition, these leaving groups could be converted to other functionals group like amines, sulphides, sulfoxides, sulfones, sulfonamaides.

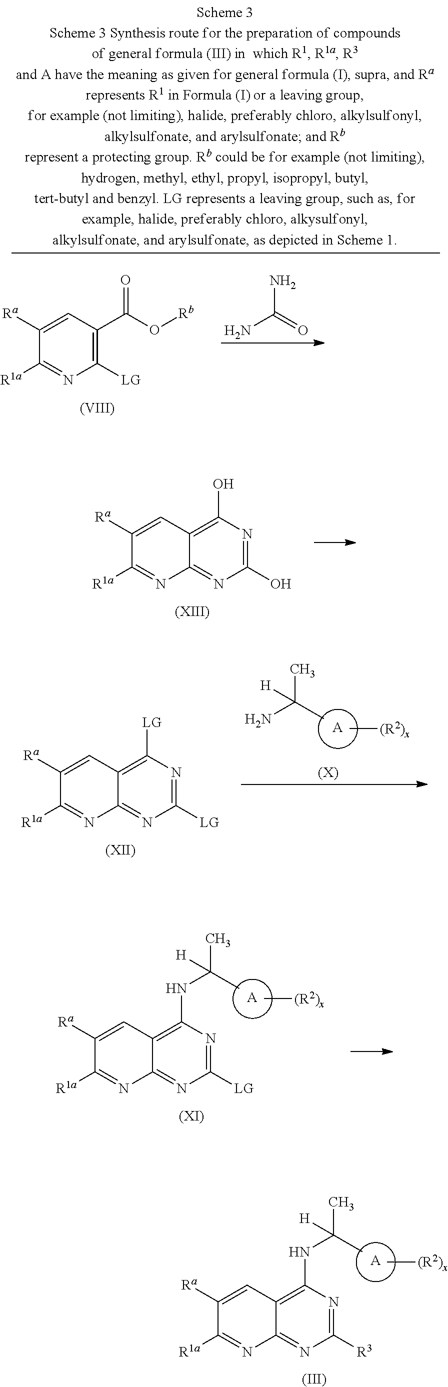

Compounds of general formula (XIII) are well-known in the public domain, commercially available or could be synthesized by known synthetic routes, for example, via formation of the heteroaromatic ring via a reaction of compounds of general formula (VIII) with urea under different conditions, see the teachings of Luo et al., CN 102584828. Or multiple step syntheses as illustrated in the teachings of Brogi et al., J. Med. Chem., 2018, 61, 2124; Bergeron et al., WO 2010014939 A1.

The transformation of compounds of general formula (XIII) to compounds of general formula (XII) are well-known in the public domain and are similarly illustrated in Scheme 2 for the conversion of (V) to (IV). For example, when LG=chloro typically trichlorophosphate or thionylchloride, with or without N,N-dimethylaniline or N,N-diisopropylethylamine, with or without an organic solvent such as, for example, toluene at elevated temperatures could be used. For examples see Cantin et al., Beoorg. Med. Chem. Lett., 2012, 2565; Bayrakdarian et al., WO 2008136756 A1; Luo et al., CN 102584828; Zhou et al., J. Med. Chem., 2015, 58, 9480.

For LG=bromo typically phosphorus oxytribromide, with or without base, with or without an organic solvent such as for example toluene at elevated temperatures could be used. For examples see Kim et al., J. Org. Chem., 2004, 69, 5638.

The transformation of compounds of general formula (XII) to compounds of general formula (XI) are well-known in the public domain and are similarly illustrated in Scheme 2 for the conversion of (IV) to (III). For example, such nucleophilic substitutions are well-documented, see the teachings of Liwicki et al., WO 2018066718 A1; Gelin et al., WO 2013016197 A1; Jiang, et al., J. Med. Chem., 2016, 59, 10498.

Compounds of general formula (XI) can be converted to compounds of general formula (II) using different synthetic methods, such as, for example, the Suzuki reaction (Liwicki et al., WO 2018066718 A1; Pulipati, et al., Synth. Commun., 2017, 47, 1142), the Stille reaction (Johnson et al., WO 2011028741 A1; Labadie et al., Bioorg. Med. Chem. Lett., 2013, 23, 5923) or other methods, see the teachings of Finlay et al., ACS Med. Chem. Letters, 2016, 7, 831.

The remaining steps within Scheme 3 follow the same route and methods as described in Scheme 2 to generate compounds of general formula (II) from compounds of general formula (III).

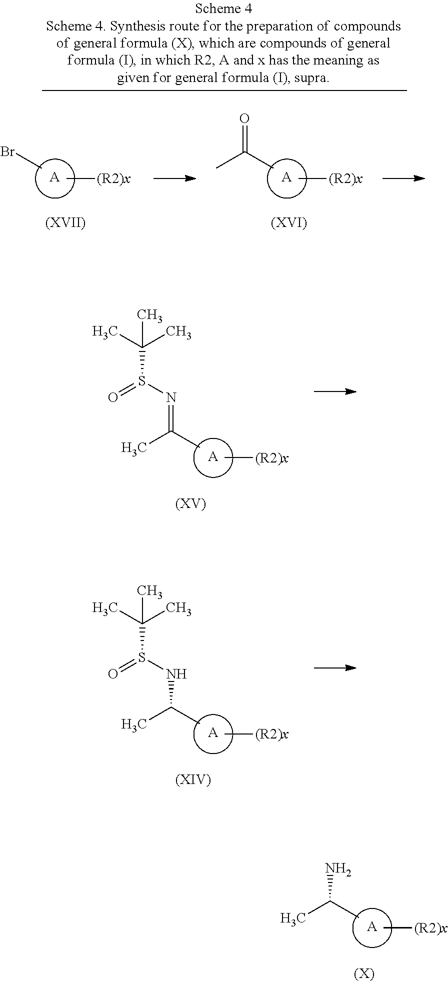

### Step XVII->XVI (Scheme 4)

Acetyl Formation

In the first step (Scheme 4) the bromo derivative XVII (which is commercially available or described in the literature) could be converted to the corresponding acetyl XVI in analogy to the numerous literature procedures. For example, the reaction can be performed using different chemistries known to those skilled in the art, for example, Grignard chemistry using magnesium in an organic solvent as for example THF; or palladium catalyzed chemistry or Stille chemistry. For such transformations see the teachings of (Grignard: Fillon et al., Tetahedron 2003, 59, 8199; Leazer et al., Org. Synth. 2005, 82, 115; Palladium: WO2005/5382; Stille: WO2019/122129 and the references therein.

### Step XVI->XV (Scheme 4)

Sulfinimine Formation

In the first step (Scheme 4) carbonyl derivative XVI (which is commercially available or described in the literature) could be converted to the corresponding sulfinimine XV in analogy to the numerous literature procedures. For example the reaction could be performed at ambient temperature using Titanium(IV) ethoxide or Titanium(IV) isopropoxide in an organic solvent as for example THF. For a review about sulfinimine chemistry see for example Chem. Rev. 2010, 110, 3600-3740; Chem. Soc. Rev. 2009, 38, 1162-1186; Tetrahedron 2004, 60, 8003 or WO2019/122129 and the references therein.

### Step XV+XIV (Scheme 4)

Formation of Sulfinamide

In the next step (Scheme 4) sulfinimine XV can be converted to the corresponding sulfinamide XIV in analogy to the numerous literature procedures. For example, the reaction can be performed using a reducing agent, for example, sodium borohydride or borane-THF, in a protic organic solvent as for example ethanol or methanol or tetrahydrofuran. Such transformations are known to those skilled in the art, see the teachings of Pan et al., Tetrahedron Asym., 2011, 22, 329; WO2019/122129; Li et al., Chem. Med. Chem., 2018, 13, 1363; Ghosh et al., Eur. J. Med. Chem., 2018, 160, 171. Alternatively, the reaction can be performed using a reducing agent, for example, diisopropylaluminium hydride, in an aprotic solvent, for example, toluene. Such transformations are known to those skilled in the art, see the teachings of WO2017/6282; Lee et al., Synlett., 2019, 30, 401.

### Step XIV->X (Scheme 4)

Formation of Amine

In the next step (Scheme 4) sulfinamide XIV can be converted to the corresponding amine X in analogy to the numerous literature procedures. For example, the reaction can be performed using acetyl chloride in a protic organic solvent as for example methanol. For a review about sulfinimine and sulfonamide chemistry see for example Chem. Rev. 2010, 110, 3600-3740; Chem. Soc. Rev. 2009, 38, 1162-1186; Tetrahedron 2004, 60, 8003 or WO2013030138 and the references therein.

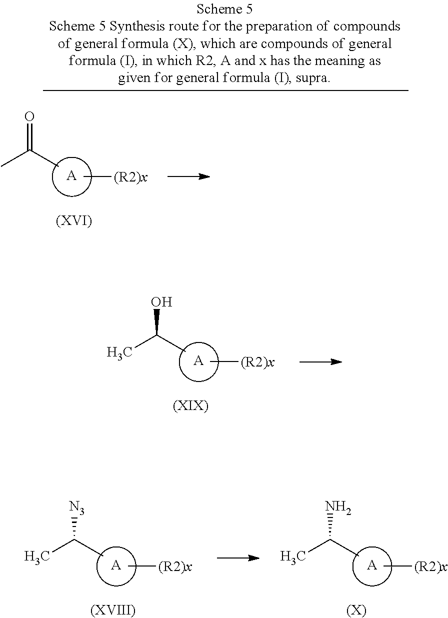

### Step XVI->XIX (Scheme 5)

### Formation of Alcohol

In the first step (Scheme 5) ketone derivative XVI (which is commercially available or described in the literature) could be converted to the corresponding chiral alcohol XIX in analogy to the numerous literature procedures. For example the enantioselective reduction could be performed using catalytic hydrogenation, with hydrogen gas under pressure with a catalyst, for example a BINAP-derived catalyst, e.g. (R)- or (S)—RUCY-Xyl-BINAP (see WO2019/122129 page 140 or WO2013/185103 page 81.)

### Step XIX->XVIII (Scheme 5)

Formation of Azide

In the next step (Scheme 5) alcohol XIX can be converted to the corresponding azide XVIII in analogy to the numerous literature procedures. For example the reaction can be performed using diphenylphosphonic azide and a base, for example, DBU, in an aprotic organic solvent as for example, toluene (see the teachings of WO2019/122129 page 144). For a review about azide chemistry see for example Chem. Rev. 1988, 88, 297.

### Step XVIII->X (Scheme 5)

Formation of Amine

In the next step (Scheme 5) azide XVIII can be converted to the corresponding amine X in analogy to the numerous literature procedures. For example, the reaction can be performed using the Staudinger reduction conditions, with a phosphine, for example, triphenyl phosphine, in water with various different organic solvents, for example methanol, ethanol or THF. Alternatively, the azide reduction can be carried out using catalytic hydrogenation methods, using a metal catalyst, for example, palladium on charcoal, under a pressurized atmosphere of hydrogen (see the teachings of WO2019/122129 page 144). For a review about azide chemistry see for example Chem. Rev. 1988, 88, 297.

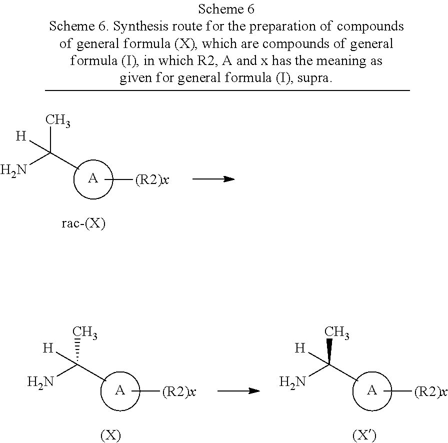

To those skilled in the art it is possible to carry out the chemical reactions described in Schemes 4 and 5, where the stereoisomers can be separated using various methods known to those skilled in the art, such as, for example, separation using chiral HPLC purification. The separation of these stereoisomers can be carried out on compounds of general formula (X).

In accordance with a further aspect, the present invention covers intermediate compounds which are useful in the preparation of compounds of the present invention of general formula (I), particularly in the methods described herein.

The present invention covers the intermediate compounds which are disclosed in the Example Section of this text, infra.

The present invention covers any sub-combination within any embodiment or aspect of the present invention of intermediate compounds of general formula (I), supra.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

Compounds of general formula (I) of the present invention demonstrate a valuable pharmacological spectrum of action which could not have been predicted. Compounds of the present invention have surprisingly been found to effectively inhibit SOS1 and it is possible therefore that said compounds be used for the treatment or prophylaxis of diseases, preferably hyper-proliferative disorders in humans and animals.

In accordance with a further aspect, the present invention covers compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, in particular hyper-proliferative disorders.

Compounds of the present invention can be utilized to inhibit, block, reduce, decrease, etc., cell proliferation and/or cell division, and/or produce apoptosis. This method comprises administering to a mammal in need thereof, including a human, an amount of a compound of general formula (I) of the present invention, or a pharmaceutically acceptable salt, isomer, polymorph, metabolite, hydrate, solvate or ester thereof, which is effective to treat the disorder.

Hyperproliferative disorders include, but are not limited to, for example: psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, invasive ductal carcinoma, invasive lobular carcinoma, ductal carcinoma in situ, and lobular carcinoma in situ. Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalmic glioma, cerebellar and cerebral astrocytoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the digestive tract include, but are not limited to, anal, colon, colorectal, oesophageal, gallbladder, gastric, pancreatic, rectal, small-intestine, and salivary gland cancers.

Tumours of the urinary tract include, but are not limited to, bladder, penile, kidney, renal pelvis, ureter, urethral and human papillary renal cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, oropharyngeal cancer, lip and oral cavity cancer and squamous cell. Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

The present invention also provides methods of treating angiogenic disorders including diseases associated with excessive and/or abnormal angiogenesis.

Inappropriate and ectopic expression of angiogenesis can be deleterious to an organism. A number of pathological conditions are associated with the growth of extraneous blood vessels. These include, for example, diabetic retinopathy, ischemic retinal-vein occlusion, and retinopathy of prematurity [Aiello et al., New Engl. J. Med., 1994, 331, 1480; Peer et al., Lab. Invest., 1995, 72, 638], age-related macular degeneration (AMD) [Lopez et al., Invest. Opththalmol. Vis. Sci., 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, compounds of general formula (I) of the present invention can be utilized to treat and/or prevent any of the aforementioned angiogenesis disorders, for example by inhibiting and/or reducing blood vessel formation; by inhibiting, blocking, reducing, decreasing, etc. endothelial cell proliferation, or other types involved in angiogenesis, as well as causing cell death or apoptosis of such cell types.

These disorders have been well characterized in humans, but also exist with a similar etiology in other mammals, and can be treated by administering pharmaceutical compositions of the present invention.

The term “treating” or “treatment” as stated throughout this document is used conventionally, for example the management or care of a subject for the purpose of combating, alleviating, reducing, relieving, improving the condition of a disease or disorder, such as a carcinoma.

The compounds of the present invention can be used in particular in therapy and prevention, i.e. prophylaxis, of tumour growth and metastases, especially in solid tumours of all indications and stages with or without pre-treatment of the tumour growth.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemotherapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In a further embodiment of the present invention, the compounds of general formula (I) of the present invention may be used to sensitize a cell to radiation, i.e. treatment of a cell with a compound of the present invention prior to radiation treatment of the cell renders the cell more susceptible to DNA damage and cell death than the cell would be in the absence of any treatment with a compound of the present invention. In one aspect, the cell is treated with at least one compound of general formula (I) of the present invention.

Thus, the present invention also provides a method of killing a cell, wherein a cell is administered one or more compounds of the present invention in combination with conventional radiation therapy.

The present invention also provides a method of rendering a cell more susceptible to cell death, wherein the cell is treated with one or more compounds of general formula (I) of the present invention prior to the treatment of the cell to cause or induce cell death. In one aspect, after the cell is treated with one or more compounds of general formula (I) of the present invention, the cell is treated with at least one compound, or at least one method, or a combination thereof, in order to cause DNA damage for the purpose of inhibiting the function of the normal cell or killing the cell.

In other embodiments of the present invention, a cell is killed by treating the cell with at least one DNA damaging agent, i.e. after treating a cell with one or more compounds of general formula (I) of the present invention to sensitize the cell to cell death, the cell is treated with at least one DNA damaging agent to kill the cell. DNA damaging agents useful in the present invention include, but are not limited to, chemotherapeutic agents (e.g. cis platin), ionizing radiation (X-rays, ultraviolet radiation), carcinogenic agents, and mutagenic agents.

In other embodiments, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of general formula (I) of the present invention is administered to a cell prior to the radiation or other induction of DNA damage in the cell. In another aspect of the invention, a compound of general formula (I) of the present invention is administered to a cell concomitantly with the radiation or other induction of DNA damage in the cell. In yet another aspect of the invention, a compound of general formula (I) of the present invention is administered to a cell immediately after radiation or other induction of DNA damage in the cell has begun. In another aspect, the cell is in vitro. In another embodiment, the cell is in vivo.

In accordance with a further aspect, the present invention covers compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, in particular hyper-proliferative disorders.

The pharmaceutical activity of the compounds according to the invention can be explained by their activity as SOS1 inhibitor.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, for the treatment or prophylaxis of diseases, in particular hyper-proliferative disorders, particularly cancer.

In accordance with a further aspect, the present invention covers the use of a compound of formula (I), described supra, or a stereoisomer, a tautomer, an N-oxide, a hydrate, a solvate, or a salt thereof, particularly a pharmaceutically acceptable salt thereof, or a mixture of same, for the prophylaxis or treatment of diseases, in particular hyperproliferative disorders, particularly cancer.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of diseases, in particular hyperproliferative disorders, particularly cancer.

In accordance with a further aspect, the present invention covers use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular hyper-proliferative disorders, particularly cancer.

In accordance with a further aspect, the present invention covers a method of treatment or prophylaxis of diseases, in particular hyper-proliferative disorders, particularly cancer, using an effective amount of a compound of general formula (I), as described supra, or stereoisomers, tautomers, N-oxides, hydrates, solvates, and salts thereof, particularly pharmaceutically acceptable salts thereof, or mixtures of same.

In accordance with a further aspect, the present invention covers pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore covers pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In accordance with another aspect, the present invention covers pharmaceutical combinations, in particular medicaments, comprising at least one compound of general formula (I) of the present invention and at least one or more further active ingredients, in particular for the treatment and/or prophylaxis of a hyperproliferative disorder, such as cancer.

Particularly, the present invention covers a pharmaceutical combination, which comprises:
one or more first active ingredients, in particular compounds of general formula (I) as defined supra, and
one or more further active ingredients, in particular a hyperproliferative disorder, such as cancer.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with known anti-hyperproliferative/anti-tumor agents/cancer therapeutics.

Examples of anti-hyperproliferative/anti-tumor agents/cancer therapeutics include:

131I-chTNT, abarelix, abiraterone, aclarubicin, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, aprepitant, arcitumomab, arglabin, arsenic trioxide, asparaginase, axitinib, azacitidine, basiliximab, belotecan, bendamustine, besilesomab, belinostat, bevacizumab, bexarotene, bicalutamide, bisantrene, bleomycin, blinatumomab, bortezomib, buserelin, bosutinib, brentuximab vedotin, busulfan, cabazitaxel, cabozantinib, calcitonine, calcium folinate, calcium levofolinate, capecitabine, capromab, carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, darolutamide, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, eculizumab, edrecolomab, elliptinium acetate, elotuzumab, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, larotrectinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, necitumumab, nedaplatin, nelarabine, neridronic acid, netupitant/palonosetron, nivolumabpentetreotide, nilotinib, nilutamide, nimorazole, nimotuzumab, nimustine, nintedanib, nitracrine, nivolumab, obinutuzumab, octreotide, ofatumumab, olaparib, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, rogaratinib, rolapitant, romidepsin, romiplostim, romurtide, roniciclib, samarium (153Sm) lexidronam, sargramostim, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

Further examples of combination partners are ATR inhibitors (e.g. BAY 1895344), DHODH inhibitors (e.g. BAY 2402234), SHP2 inhibitors (e.g. SHP099, RMC-4550, TNO155) or H-, N- or K-Ras inhibitors, including inhibitors of mutants thereof, especially K-RAS-G12C inhibitors (e.g. ARS-853, ARS-1620, AMG-510, MRTX849, MRTX1257) or farnesyl transferase inhibitors.

In particular, the present invention covers a combination of a covalent inhibitor of KRAS-G12C and a SOS1 inhibitor. It has been shown that covalent KRAS-G12C inhibitors (e.g. ARS-853 or ARS-1620) specifically bind to KRAS-G12C in the GDP-bound state, but not in the GTP-bound state (Patricelli 2016 Cancer Discovery; Janes et al. 2018 Cell), thereby trapping KRAS-G12C in its inactive GDP-bound state. In addition, it has been shown that certain RAS mutants, which usually exist in the active, GTP-bound state, are undergoing a slow intrinsic GTP hydrolysis, in particular G12C and G12D mutants of KRAS (Hunter et al. 2015 Molecular Cancer Research). It can be postulated that even those mutant RAS proteins require the activation by nucleotide exchange factors like SOS1 for full activity and tumorigenesis. Treatment with a SOS1 inhibitor is expected to shift the intracellular equilibrium of KRAS mutants towards the inactive GDP-bound state, which in turn favours binding of inhibitors of KRAS which bind preferentially to the GDP-bound state of RAS, as is the case for covalent KRAS-G12C inhibitors like ARS-853 and ARS-1620. Synergistic anti-proliferative activity in vitro has been shown for the combination of BAY-293 with ARS-853 (Hillig 2019 PNAS). Based upon standard laboratory techniques known to evaluate compounds useful for the treatment of hyper-proliferative disorders, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight.

The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### EXPERIMENTAL SECTION

NMR peak forms are stated as they appear in the spectra, possible higher order effects have not been considered.

The ¹H-NMR data of selected compounds are listed in the form of ¹H-NMR peaklists.

Therein, for each signal peak the b value in ppm is given, followed by the signal intensity, reported in round brackets. The b value-signal intensity pairs from different peaks are separated by commas. Therefore, a peaklist is described by the general form: δ₁ (intensity₁), δ₂ (intensity₂), . . . , δᵢ (intensityₙ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of the particular target compound, peaks of impurities, ¹³C satellite peaks, and/or spinning sidebands. The peaks of stereoisomers, and/or peaks of impurities are typically displayed with a lower intensity compared to the peaks of the target compound (e.g., with a purity of >90%). Such stereoisomers and/or impurities may be typical for the particular manufacturing process, and therefore their peaks may help to identify a reproduction of the manufacturing process on the basis of “by-product fingerprints”. An expert who calculates the peaks of the target compound by known methods (MestReC, ACD simulation, or by use of empirically evaluated expectation values), can isolate the peaks of the target compound as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. http://www.researchdisclosure.com/searching-disclosures, Research Disclosure Database Number 605005, 2014, 1 Aug. 2014). In the peak picking routine, as described in the Research Disclosure Database Number 605005, the parameter “MinimumHeight” can be adjusted between 1% and 4%. However, depending on the chemical structure and/or depending on the concentration of the measured compound it may be reasonable to set the parameter “MinimumHeight”<1%.

Chemical names were generated using the ACD/Name software from ACD/Labs. In some cases generally accepted names of commercially available reagents were used in place of ACD/Name generated names.

The following table 1 lists the abbreviations used in this paragraph and in the Examples section as far as they are not explained within the text body. Other abbreviations have their meanings customary per se to the skilled person.

TABLE 1
Abbreviations
The following table lists the abbreviations used herein.

| Abbreviation | Meaning |
|---|---|
| Ac₂O | acetic anhydride |
| AcOH | acetic acid (ethanoic acid) |
| aq. | aqueous |
| Boc | tert-butoxycarbonyl |
| BOP | (benzotriazol-1-yloxy)tris(dimethylamino)phosphonium |
| | hexafluorophosphate |
| br | broad (¹H-NMR signal) |
| cat. | catalytic |
| conc. | concentrated |
| CI | chemical ionisation |
| d | doublet |
| DAD | diode array detector |
| DBU | 1,8-diazabicyclo(5.4.0)undec-7-ene |
| DCC | N,N′-dicyclohexylcarbodiimide |
| DCM | dichloromethane |
| dd | double-doublet |
| DIC | N,N′-diisopropylcarbodiimide |
| DIPEA | diisopropylethylamine |
| DMA | N,N-dimethylacetamide |
| DMAP | N,N-dimethylpyridin-4-amine |
| DMF | N,N-dimethylformamide |
| DMSO | dimethylsulfoxide |
| dt | double-triplet |
| EDC | 1-ethyl-3-(3-dimethylaminopropyl)carbodiimide |
| ELSD | Evaporative Light Scattering Detector |
| EtOAc | ethyl acetate |
| EtOH | ethanol |
| eq. | equivalent |
| ESI | electrospray (ES) ionisation |
| h | hour(s) |
| HATU | 1-[bis(dimethylamino)methylene]-1H-1,2,3- |
| | triazolo[4,5-b]pyridinium 3-oxid hexafluorophosphate |
| HBTU | (o-benzotriazole-10yl)-N,N,N′,N,- |
| | tetramethyluronium hexafluorophosphate |
| HCl | hydrochloric acid |
| HPLC | high performance liquid chromatography |
| LC-MS | liquid chromatography mass spectrometry |
| m | multiplet |
| min | minute(s) |
| MeCN | acetonitrile |
| MeOH | methanol |
| MS | mass spectrometry |
| NaOtBu | sodium tert-butoxide |
| NBS | N-bromosuccinimide |
| NCS | N-chlorosuccinimide |
| NMR | nuclear magnetic resonance spectroscopy: chemical |
| | shifts (δ) are given in ppm. The chemical shifts |
| | were corrected by setting the DMSO signal to 2.50 |
| | ppm unless otherwise stated. |
| PDA | Photo Diode Array |
| Pd/C | palladium on activated charcoal |
| PdCl₂(dppf) | [1,1′-bis(diphenylphosphino)ferrocene]dichloropalladium(II) |
| Pd₂dba₃ | tris(dibenzylideneacetone)dipalladium(0) |
| PyBOP | (benzotriazol-1-yloxy)tripyrrolidinophosphonium hexafluorophosphate |
| q | quartet |
| r.t. or rt | room temperature |
| or RT | |
| rac | racemic |
| Rt | retention time (as measured either with HPLC or UPLC) in minutes |
| S | singlet |
| sat. | saturated |
| SIBX | stabilized 2-iodoxybenzoic acid |
| SM | starting material |
| SQD | Single-Quadrupole-Detector |
| t | triplet |
| T3P | propylphosphonic anhydride |
| TBAF | tetra-n-butylammonium fluoride |
| TBDMS | tert-butyldimethylsilyl |
| TBTU | N-[(1H-benzotriazol-1-yloxy)(dimethylamino)methylene]-N- |
| | methylmethanaminium tetrafluoroborate |
| td | triple-doublet |
| TEA | triethylamine |
| TFA | trifluoroacetic acid |
| THF | tetrahydrofuran |
| UPLC | ultra performance liquid chromatography |
| Xantphos | 4,5-Bis(diphenylphosphino)-9,9-dimethylxanthene |
| | (CAS 161265-03-8) |
| XPhos | 2-dicyclohexylphosphin-2′,4′,6′-triisopropylbiphenyl |
| | (CAS 564483-18-7) |
| XPhosPdG2 | chloro(2-dicyclohexylphosphino-2′,4′,6′-triisopropyl- |
| | 1,1′-biphenyl)[2-(2′-amino-1,1′-biphenyl)]palladium(II) |
| | (CAS 1310584-14-5) |

Other abbreviations have their meanings customary per se to the skilled person.

The various aspects of the invention described in this application are illustrated by the following examples which are not meant to limit the invention in any way.

The example testing experiments described herein serve to illustrate the present invention and the invention is not limited to the examples given.

### Experimental Section—General Part

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartidges KP-Sil® or KP-NH® in combination with a Biotage autopurifier system (SP4® or Isolera Four®) and eluents such as gradients of hexane/ethyl acetate or dichloromethane/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention, which is sufficiently basic, a trifluoroacetate or formate salt for example, or, in the case of a compound of the present invention, which is sufficiently acidic, an ammonium salt for example. A salt of this type can either be transformed into its free base or free acid form, respectively, by various methods known to the person skilled in the art or be used as salts in subsequent biological assays. It is to be understood that the specific form (e.g., salt, free base etc.) of a compound of the present invention as isolated and as described herein is not necessarily the only form in which said compound can be applied to a biological assay to quantify the specific biological activity.

Analytical Methods

LC-MS METHOD 1:
System: Waters Acquity UPLC-MS: Binary Solvent Manager, Sample Manager/Organizer, PDA, ELSD
Column: Acquity UPLC BEH C18 1.7 μm, 50×2.1 mm
Solvent: A=H₂O+0.1% vol. formic acid (99%) B=acetonitrile
Gradient: 0-1.6 min 1-99% B, 1.6-2 min 99% B
Flow: 0.8 mL/min
Temperature: 60° C.
Injection: 2.0 μL
Detection: DAD scan range 210-400 nm+ELSD

LC-MS METHOD 2:
System: Waters Acquity UPLC-MS: Binary Solvent Manager, Sample Manager/Organizer, PDA, ELSD
Column: Acquity UPLC BEH C18 1.7 μm, 50×2.1 mm
Solvent: A=H₂O+0.2% vol. NH₃ (32%) B=acetonitrile
Gradient: 0-1.6 min 1-99% B, 1.6-2 min 99% B
Flow: 0.8 mL/min
Temperature: 60° C.
Injection: 2.0 μL
Detection: DAD scan range 210-400 nm+ELSD

LC-MS METHOD 3:
System: SHIMADZU LCMS-2020
Column: Kinetex EVO C18 2.1 λ30 mm, 5 μm
Solvent: A=0.0375% TFA in water (V/V) B=0.01875% TFA in acetonitrile (V/V)
Gradient: 0-0.8 min 0-60% B, 0.8-1.2 min 60% B, 1.2-1.21 min 60-0% B, 1.21-1.55 min 0% B
Flow: 15 ml/min
Temperature: 40° C.
Detection: UV 220 nm & 254 nm

Preparative HPLC
a) Autopurifier: acidic conditions
System: Waters Autopurification system: Pump 2545, Sample Manager 2767, CFO, DAD 2996, ELSD 2424, SQD
Column: XBrigde C18 5.0 μm 100×30 mm
Solvent: A=H₂O+0.1% vol. formic acid (99%) B=acetonitrile
Gradient: 0-0.5 min 5% B 25 mL/min, 0.51-5.5 min 10-100% B 70 mL/min, 5.51-6.5 min 100% B 70 mL/min
Temperature: RT
Solution: max. 250 mg/max. 2.5 mL DMSO or DMF
Injection: 1×2.5 mL
Detection: DAD scan range 210-400 nm, MS ESI+, ESI−, scan range 160-1000 m/z
b) Autopurifier: basic conditions
System: Waters Autopurification system: Pump 2545, Sample Manager 2767, CFO, DAD 2996, ELSD 2424, SQD
Column: XBrigde C18 5.0 μm 100×30 mm
Solvent: A=H₂O+0.2% vol. NH₃ (32%) B=acetonitrile
Gradient: 0-0.5 min 5% B 25 mL/min, 0.51-5.5 min 10-100% B 70 mL/min, 5.51-6.5 min 100% B 70 mL/min
Temperature: RT
Solution: max. 250 mg/max. 2.5 mL DMSO or DMF
Injection: 1×2.5 mL
Detection: DAD scan range 210-400 nm, MS ESI+, ESI−, scan range 160-1000 m/z

### Experimental Section—General Procedures

### Experimental Section—Intermediates

### Intermediate 1

### 6-bromo-2-methylpyrido[2,3-d]pyrimidin-4-ol

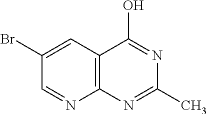

A solution of 2-amino-5-bromonicotinic acid (5.00 g, 23.0 mmol), ethanimidamide hydrochloride (7.62 g, 81.0 mmol) and sodium acetate (6.62 g, 81.0 mmol) in 2-methoxyethanol (60 mL) was heated at 150° C. for 3 days. The reaction mixture was poured into water at 0° C. and the product was collected on a sintered funnel. The titled compound (4.60 g, 83%) was washed with water and dried.

LC-MS (LC-MS METHOD 2): Rₜ=0.43 min; MS (ESIpos): m/z=242 [M+H]⁺

¹H-NMR (400 MHz, DMSO-d6) δ[ppm]: 2.392 (16.00), 2.518 (0.46), 8.554 (3.20), 8.561 (3.45), 8.986 (3.45), 8.993 (3.08), 12.659 (0.47).

### Intermediate 2

### tert-butyl (1S,4S)-5-[2-methyl-4-({(1R)-1-[2-methyl-3-(trifluoromethyl)phenyl]ethyl}amino)pyrido[2,3-d]pyrimidin-6-yl]-2,5-diazabicyclo[2.2.1]heptane-2-carboxylate

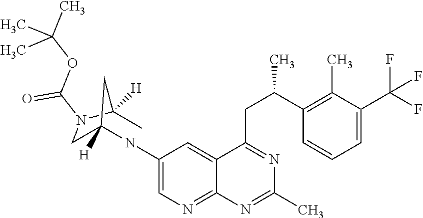

Using the method described for Example 7: Example 6 (6-bromo-2-methyl-N-{(1R)-1-[2-methyl-3-(trifluoromethyl)phenyl]ethyl}pyrido[2,3-d]pyrimidin-4-amine, 75.0 mg, 176 μmol), tert-butyl (1S,4S)-2,5-diazabicyclo[2.2.1]heptane-2-carboxylate (69.9 mg, 353 μmol), NaOtBu (37.3 mg, 388 μmol), Pd₂dba₃ (13.9 mg, 17.6 μmol), XPhos (16.8 mg, 35.3 μmol) in 1,4-dioxane (1 mL) at 100° C. overnight gave the titled compound (47.0 mg, 95% purity, 47% yield) after purification by HPLC (basic method).

LC-MS (LC-MS METHOD 2): Rₜ=1.38 min; MS (ESIpos): m/z=543 [M+H]⁺

¹H NMR (DMSO-d6) δ: 8.59 (br s, 2H), 8.37 (br d, 2H), 7.67-7.75 (m, 4H), 7.53 (d, 2H), 7.33 (m, 2H), 5.70 (br m, 2H), 4.79 (br d, 2H), 4.52 (br d, 2H), 3.65 (br m, 2H), 3.36-3.43 (m, 3H), 3.19-3.30 (m, 4H), 2.67 (m, 1H), 2.62 (s, 6H), 2.52-2.53 (m, 1H), 2.29-2.34 (m, 7H), 1.96-2.04 (m, 4H), 1.57 (d, 6H), 1.41 (s, 9H), 1.32 (s, 9H)

### Intermediate 3

### 6-(benzylsulfanyl)-2-methyl-N-{(1R)-1-[2-methyl-3-(trifluoromethyl)phenyl]ethyl}pyrido[2,3-d]pyrimidin-4-amine

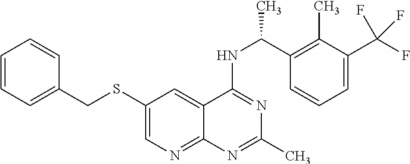 [SMILES: Cc1nc(N[C@H](C)c2cccc(C(F)(F)F)c2C)c2cc(SCc3ccccc3)cnc2n1]

To a solution of Pd₂(dba)₃ (4.31 mg, 4.70 μmol) and (9,9-dimethyl-9H-xanthene-4,5-diyl)bis(diphenylphosphine) (6.80 mg, 11.8 μmol) in 1,4-dioxane (710 μL) were added DIPEA (82 μL, 470 μmol), 6-bromo-2-methyl-N-{(1R)-1-[2-methyl-3-(trifluoromethyl)phenyl]ethyl}pyrido[2,3-d]-pyrimidin-4-amine (Example 6, 100 mg, 235 μmol) and phenylmethanethiol (30 μL, 260 μmol). The mixture was stirred at 100° C. overnight. Then, water and ethyl acetate were added, the org. phase was washed with sat. NaHCO₃ sol. and brine, filtered and concentrated under reduced pressure. Purification by flash column chromatography (hexane/ethyl acetate) gave the titled compound (108 mg, 90% purity, 88% yield).

LC-MS (LC-MS METHOD 2): Rₜ=1.45 min; MS (ESIpos): m/z=469.5 [M+H]⁺

¹H-NMR (400 MHz, DMSO-d6) δ[ppm]: 1.154 (0.89), 1.172 (1.80), 1.189 (0.89), 1.546 (4.91), 1.563 (4.91), 1.987 (3.52), 2.331 (0.67), 2.359 (16.00), 2.518 (3.73), 2.523 (2.43), 2.608 (6.02), 2.673 (0.62), 4.017 (0.79), 4.035 (0.79), 4.325 (0.70), 4.358 (3.70), 4.370 (3.65), 4.402 (0.70), 5.684 (0.74), 5.701 (1.15), 5.718 (0.74), 7.205 (0.41), 7.222 (1.49), 7.229 (0.53), 7.235 (0.72), 7.240 (1.55), 7.244 (0.88), 7.260 (1.76), 7.263 (0.95), 7.274 (1.38), 7.279 (3.80), 7.291 (0.62), 7.296 (2.19), 7.299 (1.59), 7.311 (3.28), 7.314 (3.69), 7.331 (1.78), 7.336 (1.21), 7.355 (0.68), 7.375 (1.49), 7.395 (0.92), 7.545 (1.61), 7.564 (1.29), 7.738 (1.44), 7.757 (1.28), 8.788 (1.24), 8.807 (2.00), 8.815 (8.12), 8.821 (1.32).

### Intermediate 4

### 2-methyl-4-({(1R)-1-[2-methyl-3-(trifluoromethyl)phenyl]ethyl}amino)pyrido[2,3-d]pyrimidine-6-sulfonyl chloride

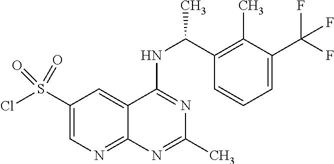 [SMILES: Cc1nc(N[C@H](C)c2cccc(C(F)(F)F)c2C)c2cc(S(=O)(=O)Cl)cnc2n1]

To a solution of 6-(benzylsulfanyl)-2-methyl-N-{(1R)-1-[2-methyl-3-(trifluoromethyl)phenyl]-ethyl}pyrido[2,3-d]pyrimidin-4-amine (Intermediate 3, 108 mg, 230 μmol) in MeCN (1.2 mL), acetic acid (130 μL) and water (58 μL) was added 1,3-dichloro-5,5-dimethylimidazolidine-2,4-dione (227 mg, 1.15 mmol) at 0° C. and the mixture was stirred for 1 h at 0° C. The mixture was diluted with dichloromethane and water, the org. phase was washed with sat. NaHCO₃ solution and brine, filtered through a hydrophobic filter and concentrated under reduced pressure. The crude product (244 mg, 42% purity, 100% yield) was used in the next step without further purification.

### Intermediate 5

### 6-bromo-2,7-dimethyl-N-{(1R)-1-[2-methyl-3-(trifluoromethyl)phenyl]ethyl}pyrido[2,3-d]pyrimidin-4-amine

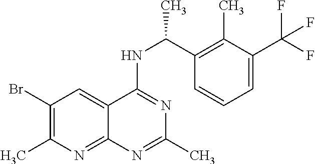 [SMILES: Cc1nc(N[C@H](C)c2cccc(C(F)(F)F)c2C)c2cc(Br)c(C)nc2n1]

To a solution of 6-bromo-2,7-dimethylpyrido[2,3-d]pyrimidin-4-ol (Intermediate 39, 500 mg, 1.97 mmol), pyBOP (1.33 g, 2.56 mmol) and (1R)-1-[2-methyl-3-(trifluoromethyl)phenyl]ethan-1-amine (480 mg, 2.36 mmol) in DMF (15 mL) was added DBU (1.2 mL, 7.9 mmol) and the reaction mixture was stirred at RT overnight. Water was added, the aq. phase was extracted with dichloromethane and the combined org. phases were dried over Na₂SO₄. Purification by flash column chromatography and recrystallization from DMSO gave the titled compound (460 mg, 53% yield).

LC-MS (LC-MS METHOD 2): Rₜ=1.36 min; MS (ESIpos): m/z=439 [M+H]⁺

¹H NMR (CHLOROFORM-d) δ: 8.48 (s, 1H), 7.60 (d, 1H), 7.51 (d, 1H), 7.20 (m, 1H), 6.75 (br d, 1H), 5.83 (m, 1H), 2.51-2.80 (m, 16H), 1.87 (br s, 2H), 1.62 (d, 3H), 1.19-1.30 (m, 1H)

### Intermediate 6

### 6-bromo-N-{(1R)-1-[3-(difluoromethyl)-2-fluorophenyl]ethyl}-2,7-dimethylpyrido[2,3-d]pyrimidin-4-amine

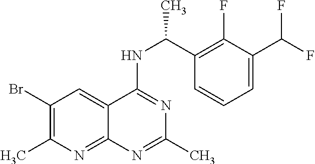 [SMILES: Cc1nc(N[C@H](C)c2cccc(C(F)F)c2F)c2cc(Br)c(C)nc2n1]

To a solution of 6-bromo-2,7-dimethylpyrido[2,3-d]pyrimidin-4-ol (Intermediate 39, 500 mg, 1.97 mmol), pyBOP (1.33 g, 2.56 mmol) and (1R)-1-[3-(difluoromethyl)-2-fluorophenyl]ethan-1-amine hydrogen chloride (1/1) (511 mg, 2.26 mmol) in DMF (17 mL) was added DBU (1.2 mL, 7.9 mmol) and the reaction mixture was stirred at RT overnight. The mixture was diluted with water and ethyl acetate, the org. phase was washed with water and brine, dried over Na₂SO₄ and concentrated under reduced pressure. Purification by flash column chromatography gave the titled compound (651 mg, 95% purity, 74% yield).

¹H NMR (DMSO-d6) δ: 9.15 (s, 4H), 8.80 (br d, 4H), 7.68 (m, 4H), 7.45-7.54 (m, 4H), 7.35-7.40 (m, 1H), 7.23-7.32 (m, 6H), 7.10 (s, 1H), 5.75 (m, 4H), 4.03 (m, 2H), 2.67-2.70 (m, 11H), 2.52-2.53 (m, 2H), 2.33-2.38 (m, 11H), 1.98-2.00 (m, 3H), 1.59 (d, 11H), 1.52 (d, 1H), 1.17 (m, 3H)

### Intermediate 7

### methyl 2-acetamido-5-bromo-6-(trifluoromethyl)pyridine-3-carboxylate

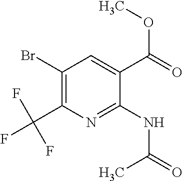 [SMILES: COC(=O)c1cc(Br)c(C(F)(F)F)nc1NC(C)=O]

A solution of methyl 2-amino-5-bromo-6-(trifluoromethyl)pyridine-3-carboxylate (5.00 g, 16.7 mmol) and DMAP (20.4 mg, 167 μmol) in Ac₂O(330 mL, 3.5 mol) was heated at 100° C. for 2 days. The mixture was concentrated under reduced pressure and used in the next step without further purification (5.7 g, 16.7 mmol).

LCMS (LC-MS METHOD 2): Rₜ=1.13 min; MS (ESIpos): m/z=343 [M+H]⁺

### Intermediate 8

### 6-bromo-2-methyl-7-(trifluoromethyl)pyrido[2,3-d]pyrimidin-4-ol

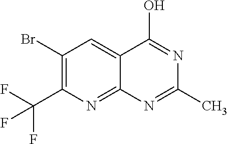 [SMILES: Cc1nc(O)c2cc(Br)c(C(F)(F)F)nc2n1]

A solution of methyl 2-acetamido-5-bromo-6-(trifluoromethyl)pyridine-3-carboxylate (Intermediate 7, 5.70 g, 16.7 mmol) in ammonium hydroxide (30%, 500 mL, 170 mmol) was stirred at rt overnight. The mixture was carefully concentrated under reduced pressure and extracted with ethyl acetate. The combined org. phases were washed with brine, dried over sodium sulfate and concentrated under reduced pressure. Purification by flash column chromatography (hexane/ethyl acetate) gave the titled compound (3.60 g, 70% yield).

LC-MS (LC-MS METHOD 2): Rₜ=0.57 min; MS (ESIneg): m/z=308 [M−H]⁻

¹H-NMR (400 MHz, DMSO-d6) δ[ppm]: 1.154 (1.02), 1.171 (2.17), 1.189 (1.04), 1.986 (3.42), 2.421 (16.00), 2.518 (0.55), 4.016 (0.74), 4.034 (0.72), 8.827 (2.66), 12.878 (0.46).

### Intermediate 9

### 6-bromo-2-methyl-N-{(1R)-1-[2-methyl-3-(trifluoromethyl)phenyl]ethyl}-7-(trifluoromethyl)pyrido[2,3-d]pyrimidin-4-amine

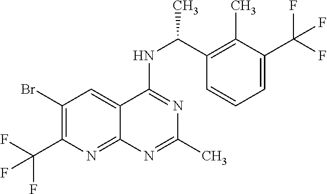 [SMILES: Cc1nc(N[C@H](C)c2cccc(C(F)(F)F)c2C)c2cc(Br)c(C(F)(F)F)nc2n1]

To a solution of 6-bromo-2-methyl-7-(trifluoromethyl)pyrido[2,3-d]pyrimidin-4-ol (Intermediate 8, 250 mg, 812 μmol), (1R)-1-[2-methyl-3-(trifluoromethyl)phenyl]ethan-1-amine (594 mg, 2.92 mmol) and pyBOP (1.65 g, 3.16 mmol) in DMF (6.3 mL) was added DBU (1.5 mL, 9.7 mmol) and the reaction mixture was heated to 50° C. overnight. The mixture was diluted with water and extracted with ethyl acetate. The organic phase was washed with water, brine, dried over Na₂SO₄, filtered, and concentrated under reduced pressure. The titled compound (258 mg, 64% yield) was obtained after purification by flash column chromatography (hexane/ethyl acetate).

LC-MS (LC-MS METHOD 2): Rₜ=1.51 min; MS (ESIpos): m/z=495 [M+H]⁺

¹H-NMR (400 MHz, DMSO-d6) δ[ppm]: 1.154 (0.84), 1.172 (1.61), 1.189 (0.74), 1.562 (4.55), 1.579 (4.46), 1.987 (3.19), 2.323 (0.53), 2.327 (0.73), 2.331 (0.51), 2.406 (16.00), 2.518 (2.68), 2.523 (1.91), 2.605 (5.08), 2.665 (0.54), 2.669 (0.74), 2.673 (0.50), 4.017 (0.63), 4.035 (0.64), 5.687 (0.66), 5.705 (1.02), 5.722 (0.65), 5.759 (0.55), 7.352 (0.57), 7.372 (1.28), 7.391 (0.77), 7.558 (1.39), 7.576 (1.11), 7.754 (1.22), 7.773 (1.09), 9.188 (1.03), 9.206 (1.00), 9.480 (3.55).

### Intermediate 10

### 6-bromo-N-{(1R)-1-[3-(2-{[tert-butyl(dimethyl)silyl]oxy}-1,1-difluoroethyl)-2-fluorophenyl]ethyl}-2-methyl-7-(trifluoromethyl)pyrido[2,3-d]pyrimidin-4-amine

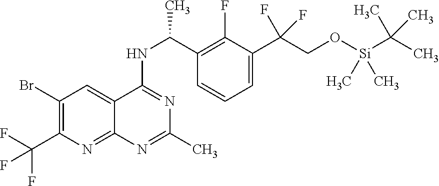 [SMILES: Cc1nc(N[C@H](C)c2cccc(C(F)(F)CO[Si](C)(C)C(C)(C)C)c2F)c2cc(Br)c(C(F)(F)F)nc2n1]

Using the method described for Intermediate 9: 6-bromo-2-methyl-7-(trifluoromethyl)pyrido[2,3-d]pyrimidin-4-ol (Intermediate 8, 115 mg, 373 μmol), (1R)-1-[3-(2-{[tert-butyl(dimethyl)silyl]oxy}-1,1-difluoroethyl)-2-fluorophenyl]ethan-1-amine (448 mg, 1.34 mmol), pyBOP (758 mg, 1.46 mmol) and DBU (670 μL, 4.5 mmol) in DMF (2.9 mL) gave the titled compound (150 mg, 64% yield) after purification by flash column chromatography (hexane/ethyl acetate).

LC-MS (LC-MS METHOD 2): Rₜ=1.72 min; MS (ESIpos): m/z=625 [M+H]⁺

¹H-NMR (400 MHz, DMSO-d6) δ[ppm]: 0.769 (0.99), 0.776 (16.00), 0.783 (0.91), 0.847 (0.97), 1.270 (0.56), 1.697 (1.31), 1.715 (1.29), 2.084 (0.96), 2.508 (4.64), 2.616 (1.28), 2.620 (0.89), 4.227 (0.64), 7.368 (0.53), 9.599 (1.09).

### Intermediate 11

### tert-butyl 6-[4-({(1R)-1-[3-(2-{[tert-butyl(dimethyl)silyl]oxy}-1,1-difluoroethyl)-2-fluorophenyl]ethyl}amino)-2-methyl-7-(trifluoromethyl)pyrido[2,3-d]pyrimidin-6-yl]-2,6-diazaspiro[3.3]heptane-2-carboxylate

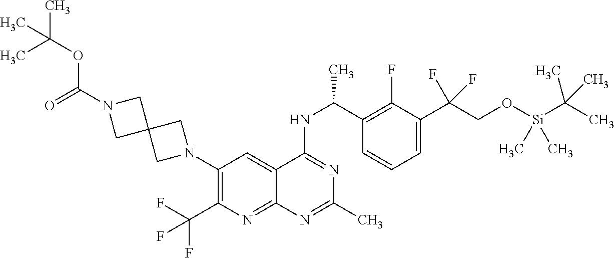 [SMILES: Cc1nc(N[C@H](C)c2cccc(C(F)(F)CO[Si](C)(C)C(C)(C)C)c2F)c2cc(N3CC4(CN(C(=O)OC(C)(C)C)C4)C3)c(C(F)(F)F)nc2n1]

A solution of 6-bromo-N-{(1R)-1-[3-(2-{[tert-butyl(dimethyl)silyl]oxy}-1,1-difluoroethyl)-2-fluorophenyl]ethyl}-2-methyl-7-(trifluoromethyl)pyrido[2,3-d]pyrimidin-4-amine (Intermediate 10, 120 mg, 192 μmol), tert-butyl 2,6-diazaspiro[3.3]heptane-2-carboxylate (45.8 mg, 231 μmol), caesium carbonate (81.5 mg, 250 μmol), palladium(II) acetate (4.32 mg, 19.2 μmol) and XPhos (14.7 mg, 30.8 μmol) in toluene (2.5 mL) was heated to 120° C. overnight. The mixture was diluted with dichloromethane, filtered, and concentrated. The titled compound (120 mg, 84% yield) was obtained after purification by flash column chromatography (hexane/ethyl acetate).

LC-MS (LC-MS METHOD 2): Rₜ=1.72 min; MS (ESIpos): m/z=742 [M+H]⁺

¹H-NMR (400 MHz, DMSO-d6) δ[ppm]: −0.021 (2.52), −0.015 (2.53), 0.037 (0.53), 0.049 (0.53), 0.754 (0.66), 0.761 (8.86), 0.768 (0.68), 0.771 (1.01), 0.778 (16.00), 0.786 (1.05), 0.828 (1.79), 0.959 (0.52), 0.976 (0.53), 1.227 (0.53), 1.233 (0.62), 1.245 (0.61), 1.251 (1.08), 1.269 (0.61), 1.311 (1.36), 1.328 (1.15), 1.453 (0.99), 1.467 (11.34), 1.680 (0.83), 1.694 (1.37), 1.711 (1.23), 2.066 (1.64), 2.401 (0.68), 2.405 (1.03), 2.413 (4.38), 2.494 (2.19), 2.596 (3.13), 2.601 (2.21), 2.743 (0.61), 2.747 (0.85), 2.752 (0.58), 4.160 (1.00), 4.182 (0.47), 4.215 (0.66), 4.303 (1.79), 5.837 (1.99), 7.004 (0.52), 7.344 (0.55), 7.494 (0.42), 8.059 (0.81), 8.074 (0.44), 8.095 (0.44).

### Intermediate 12

### tert-butyl 4-[2-methyl-4-({(1R)-1-[2-methyl-3-(trifluoromethyl)phenyl]ethyl}amino)pyrido[2,3-d]pyrimidin-6-yl]-3,6-dihydropyridine-1(2H)-carboxylate

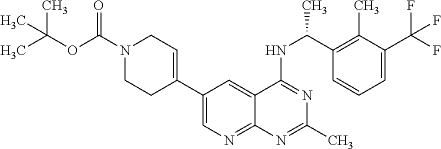 [SMILES: Cc1nc(N[C@H](C)c2cccc(C(F)(F)F)c2C)c2cc(C3=CCN(C(=O)OC(C)(C)C)CC3)cnc2n1]

Using the method described for Example 21: 6-bromo-2-methyl-N-{(1R)-1-[2-methyl-3-(trifluoromethyl)phenyl]ethyl}pyrido[2,3-d]pyrimidin-4-amine (Example 6, 100 mg, 235 μmol), tert-butyl 4-(4,4,5,5-tetramethyl-1,3,2-dioxaborolan-2-yl)-3,6-dihydropyridine-1(2H)-carboxylate (80.0 mg, 259 μmol), bis(triphenylphosphine)palladium(II) chloride (16.5 mg, 24 μmol), aq. potassium carbonate solution (180 μL, 2.0 M, 350 μmol) in 1,2-dimethoxyethane (1.0 mL) and ethanol (1.0 mL) gave the titled compound (80.0 mg, 64% yield) after purification by HPLC (basic method).

LC-MS (LC-MS METHOD 2): Rₜ=1.43 min; MS (ESIpos): m/z=528.7 [M+H]⁺

¹H NMR (DMSO-d6) δ: 9.09 (d, 1H), 8.81 (d, 1H), 8.78 (d, 1H), 7.76 (d, 1H), 7.55 (d, 1H), 7.36 (m, 1H), 6.44 (br s, 1H), 5.73 (m, 1H), 4.08 (br s, 2H), 3.62 (br m, 2H), 3.42 (s, 1H), 2.59-2.66 (m, 5H), 2.52-2.53 (m, 3H), 2.33-2.39 (m, 3H), 1.58 (d, 3H), 1.35-1.46 (m, 11H)

### Intermediate 13

### tert-butyl 7-[2-methyl-4-({(1R)-1-[2-methyl-3-(trifluoromethyl)phenyl]ethyl}amino)pyrido[2,3-d]pyrimidin-6-yl]-2-azaspiro[3.5]non-6-ene-2-carboxylate

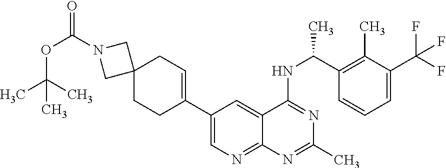 [SMILES: Cc1nc(N[C@H](C)c2cccc(C(F)(F)F)c2C)c2cc(C3=CCC4(CC3)CN(C(=O)OC(C)(C)C)C4)cnc2n1]

Using the method described for Example 21: 6-bromo-2-methyl-N-{(1R)-1-[2-methyl-3-(trifluoromethyl)phenyl]ethyl}pyrido[2,3-d]pyrimidin-4-amine (Example 6, 100 mg, 235 μmol), tert-butyl 7-(4,4,5,5-tetramethyl-1,3,2-dioxaborolan-2-yl)-2-azaspiro[3.5]non-6-ene-2-carboxylate (90.3 mg, 259 μmol), bis(triphenylphosphine)palladium(II) chloride (16.5 mg, 23.5 μmol), aqueous potassium carbonate solution (180 μL, 2.0 M, 350 μmol) in 1,2-dimethoxyethane (1.0 mL) and ethanol (1.0 mL) gave the titled compound (128 mg, 96% yield) after purification by flash column chromatography (dichloromethane/EtOH).

LC-MS (LC-MS METHOD 2): Rₜ=1.54 min; MS (ESIpos): m/z=569 [M+H]⁺

¹H-NMR (400 MHz, DMSO-d6) δ[ppm]: 1.052 (0.82), 1.066 (1.32), 1.070 (0.50), 1.156 (1.54), 1.388 (16.00), 1.565 (1.66), 1.582 (1.61), 1.944 (0.84), 1.959 (0.42), 2.363 (4.98), 2.518 (2.45), 2.523 (1.75), 2.621 (2.37), 6.373 (0.45), 7.360 (0.51), 7.537 (0.56), 7.556 (0.46), 7.756 (0.50), 7.776 (0.45), 8.754 (0.70), 8.761 (0.71), 8.812 (0.40), 9.049 (0.92), 9.055 (0.90).

### Intermediate 14

### tert-butyl 3-[2-methyl-4-({(1R)-1-[2-methyl-3-(trifluoromethyl)phenyl]ethyl}amino)pyrido[2,3-d]pyrimidin-6-yl]-2,5-dihydro-1H-pyrrole-1-carboxylate

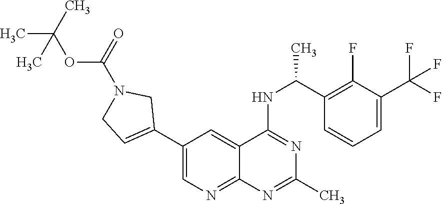 [SMILES: Cc1nc(N[C@H](C)c2cccc(C(F)(F)F)c2F)c2cc(C3=CCN(C(=O)OC(C)(C)C)C3)cnc2n1]

Using the method described for Example 21: 6-bromo-2-methyl-N-{(1R)-1-[2-methyl-3-(trifluoromethyl)phenyl]ethyl}pyrido[2,3-d]pyrimidin-4-amine (150 mg, 353 μmol), tert-butyl 3-(4,4,5,5-tetramethyl-1,3,2-dioxaborolan-2-yl)-2,5-dihydro-1H-pyrrole-1-carboxylate (115 mg, 388 μmol), bis(triphenylphosphine)palladium(II) chloride (25.8 mg, 35 μmol), aqueous potassium carbonate solution (260 μL, 2.0 M, 530 μmol) in 1,2-dimethoxyethane (1.5 mL) and ethanol (1.5 mL) at 100° C. for 4 h gave the titled compound (92.0 mg, 90% purity, 46% yield) after purification by flash column chromatography (hexane/ethyl acetate).

LC-MS (LC-MS METHOD 2): Rₜ=1.44 min; MS (ESIpos): m/z=514 [M+H]⁺

1H NMR (DMSO-d6) δ: 9.25 (d, 1H), 8.82-8.93 (m, 1H), 8.68-8.74 (m, 1H), 7.75 (br d, 1H), 7.52-7.65 (m, 2H), 7.37 (br m, 1H), 6.66 (br s, 1H), 5.72 (br m, 1H), 4.51-4.62 (m, 2H), 4.30 (br s, 2H), 2.62 (s, 3H), 2.52-2.55 (m, 6H), 2.33-2.42 (m, 3H), 1.54-1.63 (m, 3H), 1.31-1.50 (m, 9H)

### Intermediate 15

### tert-butyl 4-hydroxy-4-[2-methyl-4-({(1R)-1-[2-methyl-3-(trifluoromethyl)phenyl]ethyl}amino)pyrido[2,3-d]pyrimidin-6-yl]piperidine-1-carboxylate

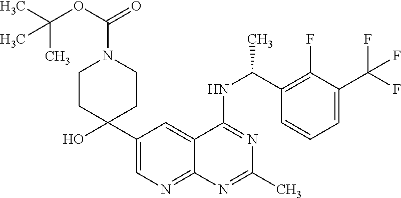 [SMILES: Cc1nc(N[C@H](C)c2cccc(C(F)(F)F)c2F)c2cc(C3(O)CCN(C(=O)OC(C)(C)C)CC3)cnc2n1]

To a solution of 6-bromo-2-methyl-N-{(1R)-1-[2-methyl-3-(trifluoromethyl)phenyl]ethyl}-pyrido[2,3-d]pyrimidin-4-amine (200 mg, 470 μmol) in THE (5.0 mL) was added sodium hydride (60%, 28.2 mg, 705 μmol) at rt and the mixture was stirred for 10 min. Then, n-butyllithium (2.5M in hexane, 280 μL) was added at −40° C. and the mixture was stirred for 45 min. Then, a solution of tert-butyl 4-oxopiperidine-1-carboxylate (281 mg, 1.41 mmol) in THE (2.0 mL) was added dropwise at −40° C. and the mixture was stirred for 1 h before being slowly warmed to 0° C. over 2 h. Then, a saturated solution of NH₄Cl was carefully added and the mixture was extracted with ethyl acetate. The combined org. phases were washed with brine, dried over Na₂SO₄, filtered, and concentrated under reduced pressure. The titled compound (4.90 mg, 95% purity, 2% yield) was obtained after purification by HPLC (basic method) and prep. TLC (dichloromethane/EtOH).

LC-MS (LC-MS METHOD 2): Rₜ=1.35 min; MS (ESIpos): m/z=546 [M+H]⁺

¹H-NMR (400 MHz, DMSO-d6) δ[ppm]: 1.427 (16.00), 1.560 (1.80), 1.578 (1.79), 1.736 (0.44), 1.771 (0.53), 2.363 (6.12), 2.518 (0.61), 2.523 (0.42), 2.625 (2.32), 3.166 (0.76), 5.723 (0.43), 7.359 (0.57), 7.533 (0.63), 7.552 (0.50), 7.760 (0.56), 7.779 (0.50), 8.837 (0.83), 8.843 (0.86), 8.897 (0.46), 8.914 (0.44), 9.089 (1.18), 9.095 (1.13).

### Intermediate 16

### tert-butyl 3-[2-methyl-4-({(1R)-1-[2-methyl-3-(trifluoromethyl)phenyl]ethyl}amino)pyrido[2,3-d]pyrimidin-6-yl]azetidine-1-carboxylate

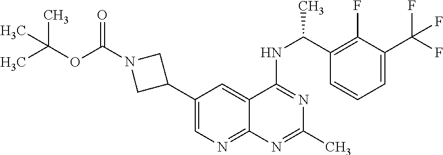 [SMILES: Cc1nc(N[C@H](C)c2cccc(C(F)(F)F)c2F)c2cc(C3CN(C(=O)OC(C)(C)C)C3)cnc2n1]

A solution of 6-bromo-2-methyl-N-{(1R)-1-[2-methyl-3-(trifluoromethyl)phenyl]ethyl}-pyrido[2,3-d]pyrimidin-4-amine (150 mg, 353 μmol), Pd(dppf)Cl₂—CH₂Cl₂ (115 mg, 141 μmol) and copper(I) iodide (13.4 mg, 70.5 μmol) in DMA (4 mL) was heated to 85° C. for 5 min. In parallel, a solution of tert-butyl 3-iodoazetidine-1-carboxylate (499 mg, 1.76 mmol) and zinc (118 mg, 1.80 mmol) in DMA (4 mL) was heated at 65° C. for 20 min. After cooling to rt, both mixtures were combined and heated at 85° C. overnight. The mixture was diluted with ethyl acetate, filtered, washed with water and brine and dried over Na₂SO₄. The mixture was concentrated under reduced pressure. Purification by flash column chromatography and prep. TLC (dichloromethane/EtOH 95:5) gave the titled compound (7.90 mg, 95% purity, 4% yield).

LC-MS (LC-MS METHOD 2): Rₜ=1.40 min; MS (ESIpos): m/z=503 [M+H]⁺

¹H-NMR (400 MHz, DMSO-d6) δ[ppm]: 0.484 (1.44), 0.504 (1.61), 0.523 (0.53), 0.884 (1.73), 0.904 (3.57), 0.924 (1.38), 1.419 (16.00), 1.570 (1.63), 1.587 (1.60), 2.327 (0.57), 2.331 (0.42), 2.364 (4.55), 2.518 (2.56), 2.523 (1.72), 2.624 (2.04), 2.669 (0.57), 2.673 (0.42), 4.026 (0.41), 4.038 (0.52), 4.049 (0.57), 4.062 (0.60), 4.075 (0.48), 7.362 (0.50), 7.539 (0.60), 7.557 (0.50), 7.755 (0.49), 7.774 (0.44), 8.830 (1.16), 8.835 (1.35).

### Intermediate 17

### 6-bromo-N-[(1R)-1-(3-{1,1-difluoro-2-methyl-2-[(triethylsilyl)oxy]propyl}-2-fluorophenyl)ethyl]-2-methylpyrido[2,3-d]pyrimidin-4-amine

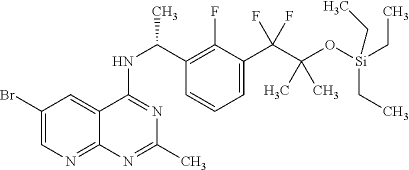 [SMILES: CC[Si](CC)(CC)OC(C)(C)C(F)(F)c1cccc([C@@H](C)Nc2nc(C)nc3ncc(Br)cc23)c1F]

6-bromo-2-methylpyrido[2,3-d]pyrimidin-4-ol (Intermediate 1, 3.89 g, 16.2 mmol) and (1R)-1-(3-{1,1-difluoro-2-methyl-2-[(triethylsilyl)oxy]propyl}-2-fluorophenyl)ethan-1-amine (16.1 g, 40% purity, 17.8 mmol) were dissolved in DMF (120 mL). PyBop (10.96 g, 21.05 mmol) and DBU (9.7 mL, 64.78 mmol) were added, and the reaction was stirred overnight at room temperature. Ethyl acetate was added to the reaction and the organic phase was washed with water twice and with sat. NaCl solution. The organic phase was dried, and the compound was purified by flash column chromatography on SiO₂ first and additionally on a basic column to give the titled compound (5.55 g, 59% yield).

LC-MS (LC-MS METHOD 2): Rₜ=1.72 min; MS (ESIpos): m/z=583 [M+H]⁺

¹H-NMR (400 MHz, DMSO-d6) δ[ppm]: 0.384 (1.14), 0.387 (1.21), 0.405 (5.21), 0.417 (0.57), 0.425 (7.62), 0.434 (0.56), 0.444 (2.69), 0.663 (7.57), 0.674 (0.88), 0.682 (16.00), 0.691 (1.14), 0.703 (5.64), 1.323 (3.86), 1.332 (4.28), 1.566 (2.95), 1.584 (2.98), 2.367 (11.84), 2.518 (5.24), 2.523 (3.80), 3.321 (0.48), 5.753 (0.44), 5.770 (0.71), 5.788 (0.46), 7.199 (0.41), 7.218 (1.02), 7.238 (0.73), 7.272 (0.40), 7.276 (0.50), 7.293 (0.64), 7.622 (0.62), 8.790 (0.76), 8.808 (0.74), 8.991 (2.74), 8.998 (2.85), 9.199 (2.02), 9.205 (1.92).

### Intermediate 18

### 1-(3-{(1R)-1-[(6-bromo-2-methylpyrido[2,3-d]pyrimidin-4-yl)amino]ethyl}-2-fluorophenyl)-1,1-difluoro-2-methylpropan-2-ol

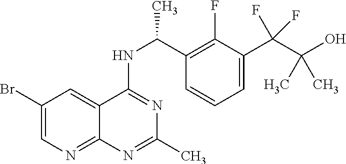 [SMILES: Cc1nc(N[C@H](C)c2cccc(C(F)(F)C(C)(C)O)c2F)c2cc(Br)cnc2n1]

6-bromo-N-[(1R)-1-(3-{1,1-difluoro-2-methyl-2-[(triethylsilyl)oxy]propyl}-2-fluorophenyl)ethyl]-2-methylpyrido[2,3-d]pyrimidin-4-amine (Intermediate 17, 1.00 g, 1.71 mmol) and triethyl silane (27 μL, 170 μmol) were dissolved in dichloromethane (15 mL). At RT, TFA (2.0 mL, 26 mmol) was added dropwise. The mixture was stirred overnight at RT. Toluene was added to the mixture and the solvent was evaporated. The crude was purified by flash column chromatography on silica to give the titled compound (815 mg, quant).

LC-MS (LC-MS METHOD 2): Rₜ=1.15 min; MS (ESIpos): m/z=469 [M+H]⁺

¹H-NMR (400 MHz, DMSO-d6) δ[ppm]: 1.154 (1.56), 1.171 (3.19), 1.189 (1.97), 1.202 (6.12), 1.226 (6.30), 1.563 (4.72), 1.580 (4.71), 1.986 (5.13), 2.326 (1.02), 2.332 (0.76), 2.368 (16.00), 2.518 (4.80), 2.522 (3.15), 2.669 (1.03), 2.673 (0.74), 4.017 (1.18), 4.034 (1.19), 5.338 (2.22), 5.731 (0.73), 5.748 (1.14), 5.766 (0.72), 7.197 (0.70), 7.216 (1.68), 7.235 (1.06), 7.299 (0.64), 7.303 (0.73), 7.321 (1.06), 7.336 (0.51), 7.340 (0.46), 7.588 (0.59), 7.604 (1.04), 7.620 (0.54), 8.803 (1.25), 8.821 (1.19), 8.995 (3.68), 9.002 (3.64), 9.196 (3.17), 9.202 (2.98).

### Intermediate 19

### tert-butyl 4-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-3,6-dihydropyridine-1(2H)-carboxylate

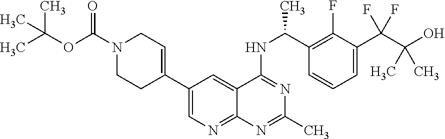 [SMILES: Cc1nc(N[C@H](C)c2cccc(C(F)(F)C(C)(C)O)c2F)c2cc(C3=CCN(C(=O)OC(C)(C)C)CC3)cnc2n1]

A mixture of 1-(3-{(1R)-1-[(6-bromo-2-methylpyrido[2,3-d]pyrimidin-4-yl)amino]ethyl}-2-fluorophenyl)-1,1-difluoro-2-methylpropan-2-ol (Intermediate 18, 200 mg, 426 μmol), tert-butyl 4-(4,4,5,5-tetramethyl-1,3,2-dioxaborolan-2-yl)-3,6-dihydropyridine-1(2H)-carboxylate (158 mg, 511 μmol), bis(triphenylphosphine)palladium(II) dichloride (29.9 mg, 43 μmol) and potassium carbonate (88.3 mg, 639 μmol) in DME (1.9 mL) and EtOH (1.9 mL) was purged with argon and heated in a microwave at 100° C. for 6 h. The mixture was filtered through a syringe filter, concentrated under reduced pressure and purified by HPLC (basic method) to give the titled compound (152 mg, 62% yield) as white solid.

LC-MS (LC-MS METHOD 2): Rₜ=1.27 min; MS (ESIpos): m/z=573 [M+H]⁺

¹H NMR (400 MHz, DMSO-d₆) δ ppm 9.10 (d) 8.80 (d) 8.72 (d) 7.60 (t) 7.32 (t) 7.22 (t) 6.44 (br s) 5.80 (quin) 5.34 (s) 4.09 (br s) 3.62 (br t) 2.57-2.68 (m) 2.52-2.57 (m) 2.37 (s) 1.60 (d) 1.44 (s) 1.22 (d)

### Intermediate 20

### tert-butyl 3-(4-{[(1R)-1-(3-{1,1-difluoro-2-methyl-2-[(triethylsilyl)oxy]propyl}-2-fluorophenyl)ethyl]amino}-2-methylpyrido[2,3-d]pyrimidin-6-yl)-3,6-diazabicyclo[3.1.1]heptane-6-carboxylate

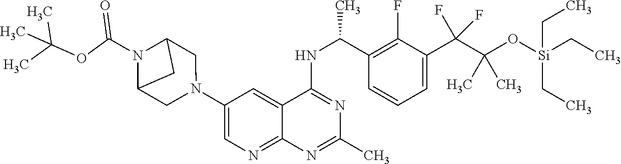 [SMILES: CC[Si](CC)(CC)OC(C)(C)C(F)(F)c1cccc([C@@H](C)Nc2nc(C)nc3ncc(N4CC5CC(C4)N5C(=O)OC(C)(C)C)cc23)c1F]

6-bromo-N-[(1R)-1-(3-{1,1-difluoro-2-methyl-2-[(triethylsilyl)oxy]propyl}-2-fluorophenyl)ethyl]-2-methylpyrido[2,3-d]pyrimidin-4-amine (Intermediate 17, 100 mg, 171 μmol) and tert-butyl 3,6-diazabicyclo[3.1.1]heptane-6-carboxylate (51.0 mg, 257 μmol) were dissolved in dioxane (2.5 mL). Sodium tert-butoxide (65.9 mg, 0.034 mmol) was added, followed by XPhos (16.3 mg, 34.3 μmol). The atmosphere was exchanged to argon and Pd₂(dba)₃ (15.7 mg, 17.1 μmol) was added. The mixture was heated to 100° C. overnight. The mixture was cooled to RT and diluted with dichloromethane and filtered. The solvent was evaporated, and the residue was purified by flash column chromatography on silica to give the titled compound (61.1 mg, 51% yield).

LC-MS (LC-MS METHOD 2): Rₜ=1.70 min; MS (ESIpos): m/z=701 [M+H]⁺

¹H NMR (400 MHz, DMSO-d₆) δ ppm 8.70 (d) 8.38 (br d) 7.77-7.83 (m) 7.55-7.65 (m) 7.16-7.32 (m) 5.77-5.87 (m) 4.28 (br d) 3.89-4.04 (m) 3.38-3.53 (m) 2.54-2.68 (m) 2.28-2.45 (m) 1.42-1.65 (m) 1.31-1.35 (m) 1.27 (s) 0.61-0.86 (m) 0.34-0.51 (m)

### Intermediate 21

### tert-butyl (1S,4S)-5-(4-{[(1R)-1-(3-{1,1-difluoro-2-methyl-2-[(triethylsilyl)oxy]propyl}-2-fluorophenyl)ethyl]amino}-2-methylpyrido[2,3-d]pyrimidin-6-yl)-2,5-diazabicyclo[2.2.1]heptane-2-carboxylate

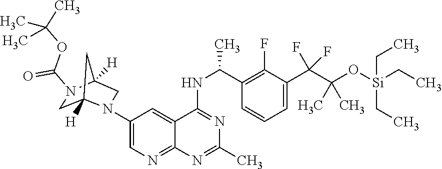 [SMILES: CC[Si](CC)(CC)OC(C)(C)C(F)(F)c1cccc([C@@H](C)Nc2nc(C)nc3ncc(N4C[C@@H]5C[C@H]4CN5C(=O)OC(C)(C)C)cc23)c1F]

6-bromo-N-[(1R)-1-(3-{1,1-difluoro-2-methyl-2-[(triethylsilyl)oxy]propyl}-2-fluorophenyl)ethyl]-2-methylpyrido[2,3-d]pyrimidin-4-amine (Intermediate 17, 100 mg, 171 μmol) and tert-butyl (1S,4S)-2,5-diazabicyclo[2.2.1]heptane-2-carboxylate (51.0 mg, 257 μmol) were dissolved in dioxane (2.5 mL). Sodium tert-butoxide (65.9 mg, 0.685 mml) was added, followed by XPhos (16.3 mg, 34.3 μmol). The atmosphere was exchanged to argon and Pd₂(dba)₃ (15.7 mg, 17.1 μmol) was added, and the mixture was heated to 100° C. overnight. The mixture was cooled to RT and diluted with dichloromethane and filtered. The solvent was evaporated. The residue was purified by flash column chromatography on silica to give the titled compound (76.3 mg, 64% yield).

LC-MS (LC-MS METHOD 2): Rₜ=1.70 min; MS (ESIpos): m/z=701 [M+H]⁺

¹H NMR (400 MHz, DMSO-d₆) δ ppm 8.61 (br s) 8.25 (br d) 7.71 (br d) 7.53-7.62 (m) 7.27 (t) 7.15-7.24 (m) 5.79 (quin) 4.76-4.83 (m) 4.52 (br d) 3.61-3.70 (m) 3.37-3.45 (m) 3.18-3.31 (m) 2.52-2.57 (m) 2.34-2.46 (m) 2.28-2.31 (m) 1.95-2.05 (m) 1.44-1.65 (m) 1.41 (s) 1.29-1.36 (m) 0.63-0.74 (m) 0.42 (q)

### Intermediate 22

### tert-butyl (1 R,4R)-5-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-2,5-diazabicyclo[2.2.1]heptane-2-carboxylate

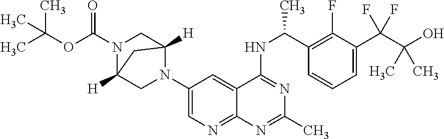 [SMILES: Cc1nc(N[C@H](C)c2cccc(C(F)(F)C(C)(C)O)c2F)c2cc(N3C[C@H]4C[C@@H]3CN4C(=O)OC(C)(C)C)cnc2n1]

1-(3-{(1R)-1-[(6-bromo-2-methylpyrido[2,3-d]pyrimidin-4-yl)amino]ethyl}-2-fluorophenyl)-1,1-difluoro-2-methylpropan-2-ol (Intermediate 18, 100 mg, 213 μmol) and tert-butyl (1 R,4R)-2,5-diazabicyclo[2.2.1]heptane-2-carboxylate (50.7 mg, 256 μmol) were dissolved in dioxane (3.0 mL). Sodium tert-butoxide (28.7 mg, 0.298 mml) was added, followed by XPhos (20.3 mg, 42.6 μmol). The atmosphere was exchanged to argon and Pd₂dba₃ (19.5 mg, 21.3 μmol) was added. The mixture was heated to 100° C. overnight. The mixture was cooled to RT and sat. brine was added, followed by ethyl acetate. The aq. Phase was extracted with ethyl acetate. The organic phase was dried, and the solvent was evaporated. The residue was purified by flash column chromatography on silica to give the titled compound (45 mg, 36% yield).

LC-MS (LC-MS METHOD 2): Rₜ=1.20 min; MS (ESIpos): m/z=587 [M+H]⁺

¹H NMR (400 MHz, DMSO-d₆) δ ppm 8.63 (d) 7.70-7.79 (m) 7.56-7.63 (m) 7.32 (t) 7.19-7.26 (m) 5.75-5.83 (m) 5.32-5.36 (m) 4.79 (br d) 4.53 (br d) 3.62-3.72 (m) 3.36-3.45 (m) 3.17-3.30 (m) 2.52-2.54 (m) 2.33-2.46 (m) 1.95-2.06 (m) 1.55-1.66 (m) 1.40 (s) 1.33 (s) 1.21 (br d)

### Intermediate 23

### N-[(1R)-1-(3-{1,1-difluoro-2-methyl-2-[(triethylsilyl)oxy]propyl}-2-fluorophenyl)ethyl]-2-methyl-6-(4-methylpiperazin-1-yl)pyrido[2,3-d]pyrimidin-4-amine

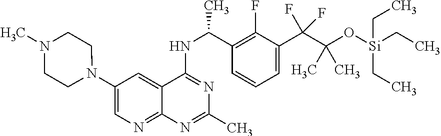 [SMILES: CC[Si](CC)(CC)OC(C)(C)C(F)(F)c1cccc([C@@H](C)Nc2nc(C)nc3ncc(N4CCN(C)CC4)cc23)c1F]

6-bromo-N-[(1R)-1-(3-{1,1-difluoro-2-methyl-2-[(triethylsilyl)oxy]propyl}-2-fluorophenyl)ethyl]-2-methylpyrido[2,3-d]pyrimidin-4-amine (Intermediate 17, 57.0 mg, 97.7 μmol) and 1-methylpiperazine (33 μL, 290 μmol) were dissolved in dioxane (1.5 mL). Sodium tert-butoxide (37.5 mg, 0.391 mmol) was added, followed by XPhos (9.31 mg, 19.5 μmol). The atmosphere was exchanged to argon and Pd₂dba₃ (8.94 mg, 9.77 μmol) was added, and the mixture was heated to 100° C. overnight. The mixture was cooled to RT and dichloromethane was added and filtered. The solvent was evaporated. The crude was purified by preparative TLC using dichloromethane/MeOH 1/1 as eluent to give the titled compound (36.0 mg, 61% yield).

LC-MS (LC-MS METHOD 2): Rₜ=1.61 min; MS (ESIpos): m/z=603 [M+H]⁺

¹H NMR (400 MHz, DMSO-d₆) δ ppm 8.85 (d) 8.41 (d) 8.04 (d) 7.59 (t) 7.28 (t) 7.21 (t) 5.76-5.84 (m) 3.38-3.46 (m) 3.31-3.31 (m) 2.67 (dt) 2.52-2.55 (m) 2.30-2.34 (m) 2.26 (s) 1.47-1.64 (m) 1.21-1.38 (m) 0.63-0.75 (m) 0.38-0.48 (m)

### Intermediate 24

### 1-[4-(4-{[(1R)-1-(3-{1,1-difluoro-2-methyl-2-[(triethylsilyl)oxy]propyl}-2-fluorophenyl)ethyl]amino}-2-methylpyrido[2,3-d]pyrimidin-6-yl)piperazin-1-yl]ethan-1-one

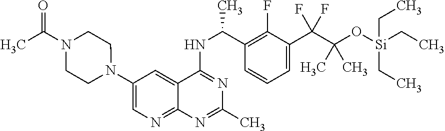 [SMILES: CC[Si](CC)(CC)OC(C)(C)C(F)(F)c1cccc([C@@H](C)Nc2nc(C)nc3ncc(N4CCN(C(C)=O)CC4)cc23)c1F]

6-bromo-N-[(1R)-1-(3-{1,1-difluoro-2-methyl-2-[(triethylsilyl)oxy]propyl}-2-fluorophenyl)ethyl]-2-methylpyrido[2,3-d]pyrimidin-4-amine (Intermediate 17, 138 mg, 236 μmol) and 1-(piperazin-1-yl)ethan-1-one (60.6 mg, 473 μmol) were dissolved in dioxane (3.5 mL). Sodium tert-butoxide (90.9 mg, 946 μmol) and XPhos (22.5 mg, 47.3 μmol) were added, the atmosphere was exchanged to argon and Pd₂dba₃ (21.7 mg, 23.6 μmol) was added. The mixture was heated to 100° C. overnight. The mixture was cooled to RT and dichloromethane was added and the mixture was filtered. The solvent was evaporated, and the residue was purified by flash column chromatography on silica to give the titled compound (46.0 mg, 31% yield).

LC-MS (LC-MS METHOD 2): Rₜ=1.54 min; MS (ESIpos): m/z=632 [M+H]⁺

¹H NMR (400 MHz, DMSO-d₆) δ ppm 8.89 (d) 8.43 (d) 8.09 (d) 7.59 (br t) 7.25-7.31 (m) 7.21 (t) 5.77-5.84 (m) 3.66 (q) 3.36-3.47 (m) 3.23-3.30 (m) 2.67 (dt) 2.52-2.52 (m) 2.32-2.43 (m) 2.32 (s) 2.08 (s) 1.59 (d) 1.33 (br d) 0.66-0.73 (m) 0.38-0.48 (m)

### Intermediate 25

### tert-butyl 6-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-2,6-diazaspiro[3.3]heptane-2-carboxylate

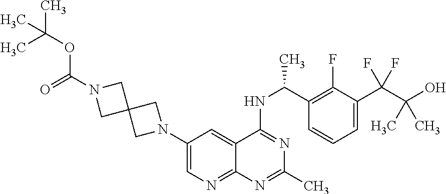 [SMILES: Cc1nc(N[C@H](C)c2cccc(C(F)(F)C(C)(C)O)c2F)c2cc(N3CC4(CN(C(=O)OC(C)(C)C)C4)C3)cnc2n1]

1-(3-{(1R)-1-[(6-bromo-2-methylpyrido[2,3-d]pyrimidin-4-yl)amino]ethyl}-2-fluorophenyl)-1,1-difluoro-2-methylpropan-2-ol (Intermediate 18, 100 mg, 213 μmol) and tert-butyl 2,6-diazaspiro[3.3]heptane-2-carboxylate (50.7 mg, 256 μmol) were dissolved in dioxane (3.0 mL). Sodium tert-butoxide (28.7 mg, 298 μmol) and XPhos (20.3 mg, 42.6 μmol) were added, the atmosphere was exchanged to argon and Pd₂dba₃ (19.5 mg, 21.3 μmol) was added. The mixture was heated to 100° C. overnight. Sat. brine and ethyl acetate were added. The aq. phase was extracted with ethyl acetate. The organic phase was dried, and the solvent was evaporated. The residue was purified by flash column chromatography on silica to give the titled compound (69.0 mg, 95% purity, 52% yield).

LC-MS (LC-MS METHOD 2): Rₜ=1.22 min; MS (ESIpos): m/z=587 [M+H]⁺

¹H NMR (400 MHz, DMSO-d₆) δ ppm 8.37 (d) 7.74 (d) 7.59 (br t) 7.32 (t) 7.24 (t) 5.76-5.85 (m) 5.34 (s) 4.12-4.19 (m) 4.08 (br s) 2.52-2.55 (m) 2.33-2.40 (m) 1.61 (d) 1.39 (s) 1.15-1.30 (m)

### Intermediate 26

### 2-(4-{[(1R)-1-(3-{1,1-difluoro-2-methyl-2-[(triethylsilyl)oxy]propyl}-2-fluorophenyl)ethyl]amino}-2,7-dimethylpyrido[2,3-d]pyrimidin-6-yl)-1 lambda⁶,2-thiazolidine-1,1-dione

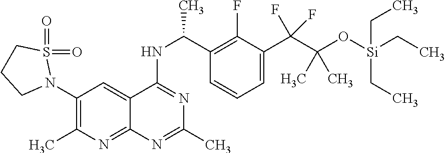 [SMILES: CC[Si](CC)(CC)OC(C)(C)C(F)(F)c1cccc([C@@H](C)Nc2nc(C)nc3nc(C)c(N4CCCS4(=O)=O)cc23)c1F]

Using the method described for Example 33: 6-bromo-N-[(1R)-1-(3-{1,1-difluoro-2-methyl-2-[(triethylsilyl)oxy]propyl}-2-fluorophenyl)ethyl]-2,7-dimethylpyrido[2,3-d]pyrimidin-4-amine (Intermediate 40, 65.0 mg, 109 μmol), 1lambda⁶,2-thiazolidine-1,1-dione (15.8 mg, 131 μmol), copper(I) iodide (4.14 mg, 21.8 μmol), trans-N,N-dimethylcyclohexane-1,2-diamine (3.09 mg, 21.8 μmol) and potassium carbonate (30.1 mg, 218 μmol) in 1,4-dioxane (1.3 ml) at 100° C. overnight gave the titled compound, which was directly used for the following step.

### Intermediate 27

### 1-[6-(4-{[(1R)-1-(3-{1,1-difluoro-2-methyl-2-[(triethylsilyl)oxy]propyl}-2-fluorophenyl)ethyl]amino}-2,7-dimethylpyrido[2,3-d]pyrimidin-6-yl)-2,6-diazaspiro[3.3]heptan-2-yl]ethan-1-one

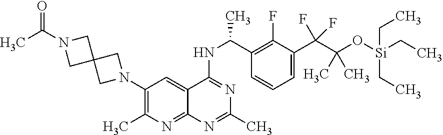 [SMILES: CC[Si](CC)(CC)OC(C)(C)C(F)(F)c1cccc([C@@H](C)Nc2nc(C)nc3nc(C)c(N4CC5(CN(C(C)=O)C5)C4)cc23)c1F]

Using the method described for Example 7, Intermediate 40 (6-bromo-N-[(1R)-1-(3-{1,1-difluoro-2-methyl-2-[(triethylsilyl)oxy]propyl}-2-fluorophenyl)ethyl]-2,7-dimethylpyrido[2,3-d]pyrimidin-4-amine, 100 mg, 167 μmol), oxalic acid/1-(2,6-diazaspiro[3.3]heptan-2-yl)ethan-1-one (½) (93.0 mg, 251 μmol), sodium tert-butoxide (80.4 mg, 837 μmol), XPhos (16.0 mg, 33.5 μmol), Pd₂(dba)₃ (15.3 mg, 16.7 μmol) in 1,4-dioxane (2.0 ml) gave the titled compound, 49.0 mg (45% yield) after preparative TLC with dichloromethane/methanol (1:1) as eluent.

LC-MS (LC-MS METHOD 2): Rₜ=1.56 min; MS (ESIpos): m/z=657 [M+H]⁺

¹H NMR (400 MHz, DMSO-d₆) δ ppm 8.36 (br d) 7.65 (s) 7.58 (t) 7.24-7.31 (m) 7.12-7.24 (m) 5.74-5.84 (m) 4.34 (s) 4.12-4.26 (m) 4.05 (s) 2.52-2.62 (m) 2.45-2.47 (m) 2.26-2.32 (m) 1.67-1.81 (m) 1.58 (d) 1.34 (br d) 0.63-0.76 (m) 0.37-0.50 (m).

### Intermediate 28

### tert-butyl 4-[2-methyl-4-({(1R)-1-[2-methyl-3-(trifluoromethyl)phenyl]ethyl}amino)pyrido[2,3-d]pyrimidin-6-yl]-3-oxopiperazine-1-carboxylate

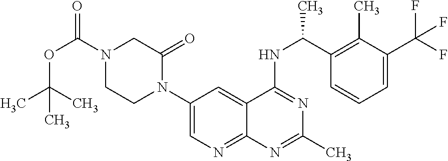 [SMILES: Cc1nc(N[C@H](C)c2cccc(C(F)(F)F)c2C)c2cc(N3CCN(C(=O)OC(C)(C)C)CC3=O)cnc2n1]

Using the method described for Example 33: Intermediate 6 (6-bromo-2-methyl-N-{(1R)-1-[2-methyl-3-(trifluoromethyl)phenyl]ethyl}pyrido[2,3-d]pyrimidin-4-amine (75.0 mg, 176 μmol), tert-butyl 3-oxopiperazine-1-carboxylate (35.3 mg, 176 μmol), copper(I) iodide (10.1 mg, 52.9 μmol), trans-N,N-dimethylcyclohexane-1,2-diamine (7.53 mg, 52.9 μmol) and K₃PO₄ (75 mg, 353 μmol) in 1,4-dioxane (1 ml) at 100° C. overnight gave the titled compound, which was directly used in the following step.

### Intermediate 29

### tert-butyl 4-(4-{[(1R)-1-(3-{1,1-difluoro-2-methyl-2-[(triethylsilyl)oxy]propyl}-2-fluorophenyl)ethyl]amino}-2,7-dimethylpyrido[2,3-d]pyrimidin-6-yl)-5-methyl-3,6-dihydropyridine-1(2H)-carboxylate

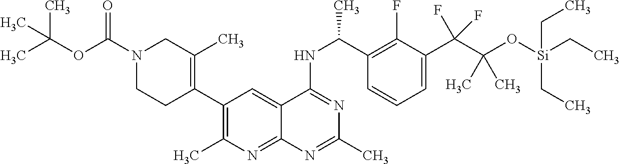 [SMILES: CC[Si](CC)(CC)OC(C)(C)C(F)(F)c1cccc([C@@H](C)Nc2nc(C)nc3nc(C)c(C4=C(C)CN(C(=O)OC(C)(C)C)CC4)cc23)c1F]

Using the method described for Example 21, Intermediate 40 (6-bromo-N-[(1R)-1-(3-{1,1-difluoro-2-methyl-2-[(triethylsilyl)oxy]propyl}-2-fluorophenyl)ethyl]-2,7-dimethylpyrido[2,3-d]pyrimidin-4-amine (250 mg, 418 μmol), tert-butyl 5-methyl-4-(4,4,5,5-tetramethyl-1,3,2-dioxaborolan-2-yl)-3,6-dihydropyridine-1(2H)-carboxylate (149 mg, 460 μmol), bis(triphenylphosphine)-palladium(II) chloride (59 mg, 84 μmol) and potassium carbonate (86.7 mg, 628 μmol) in 1,2-dimethoxyethane (2.0 ml) and ethanol (2.0 ml) gave the titled compound (41.1 mg (14% yield) after flash column chromatography on silica using dichloromethane and ethanol (9:1) as eluent.

LC-MS (LC-MS METHOD 2): Rₜ=1.83 min; MS (ESIpos): m/z=714 [M+H]⁺

¹H-NMR (400 MHz, DMSO-d6) δ[ppm]: 0.381 (0.44), 0.385 (0.48), 0.391 (0.55), 0.395 (0.61), 0.402 (1.68), 0.413 (1.92), 0.423 (2.45), 0.433 (2.59), 0.442 (1.25), 0.452 (1.09), 0.665 (3.39), 0.668 (3.40), 0.684 (6.38), 0.687 (6.59), 0.704 (2.44), 0.707 (2.40), 1.322 (3.32), 1.335 (1.66), 1.412 (1.48), 1.424 (1.63), 1.448 (16.00), 1.541 (1.91), 1.559 (1.89), 2.270 (0.59), 2.323 (0.59), 2.327 (0.84), 2.331 (0.77), 2.339 (3.74), 2.345 (3.51), 2.440 (5.75), 2.518 (2.78), 2.523 (1.90), 2.665 (0.68), 2.669 (0.71), 2.673 (0.49), 7.204 (0.40), 7.218 (0.41), 7.278 (0.42), 7.595 (0.40), 8.477 (0.93), 8.493 (0.90).

### Intermediate 30

### 1-[4-(4-{[(1R)-1-(3-{1,1-difluoro-2-methyl-2-[(triethylsilyl)oxy]propyl}-2-fluorophenyl)ethyl]amino}-2,7-dimethylpyrido[2,3-d]pyrimidin-6-yl)piperazin-1-yl]ethan-1-one

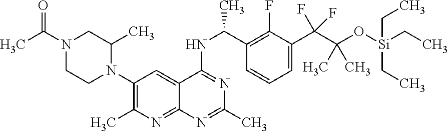 [SMILES: CC[Si](CC)(CC)OC(C)(C)C(F)(F)c1cccc([C@@H](C)Nc2nc(C)nc3nc(C)c(N4CCN(C(C)=O)CC4C)cc23)c1F]

Using the method described for Example 7, Intermediate 40 (6-bromo-N-[(1R)-1-(3-{1,1-difluoro-2-methyl-2-[(triethylsilyl)oxy]propyl}-2-fluorophenyl)ethyl]-2,7-dimethylpyrido[2,3-d]pyrimidin-4-amine (100 mg, 167 μmol), 1-(piperazin-1-yl)ethan-1-one (32.2 mg, 251 μmol), sodium tert-butoxide (69.5 mg, 723 μmol), XPhos (16.0 mg, 33.5 μmol), Pd₂(dba)₃ (15.3 mg, 16.7 μmol) in 1,4-dioxane (1.5 ml) gave the titled compound (31.0 mg, 29% yield) after preparative TLC using dichloromethane/ethanol (9:1) as eluent.

LC-MS (LC-MS METHOD 2): Rₜ=1.59 min; MS (ESIpos): m/z=645 [M+H]⁺

¹H NMR (400 MHz, DMSO-d₆) δ ppm 8.45 (d) 8.32 (s) 7.54-7.61 (m) 7.12-7.31 (m) 5.74-5.84 (m) 3.60-3.71 (m) 3.37-3.49 (m) 3.16-3.30 (m) 2.87-3.00 (m) 2.67 (dt) 2.60 (s) 2.52-2.52 (m) 2.29-2.36 (m) 2.07 (s) 2.01-2.07 (m) 1.46-1.64 (m) 1.33 (br d) 0.64-0.73 (m) 0.36-0.49 (m).

### Intermediate 31

### tert-butyl (1S,4S)-5-(4-{[(1R)-1-(3-{1,1-difluoro-2-methyl-2-[(triethylsilyl)oxy]propyl}-2-fluorophenyl)ethyl]amino}-2,7-dimethylpyrido[2,3-d]pyrimidin-6-yl)-2,5-diazabicyclo[2.2.1]heptane-2-carboxylate

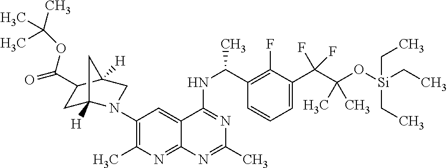 [SMILES: CC[Si](CC)(CC)OC(C)(C)C(F)(F)c1cccc([C@@H](C)Nc2nc(C)nc3nc(C)c(N4C[C@@H]5C[C@H]4CC5C(=O)OC(C)(C)C)cc23)c1F]

Using the method described for Example 7, Intermediate 40 (6-bromo-N-[(1R)-1-(3-{1,1-difluoro-2-methyl-2-[(triethylsilyl)oxy]propyl}-2-fluorophenyl)ethyl]-2,7-dimethylpyrido[2,3-d]pyrimidin-4-amine (135 mg, 244 μmol), tert-butyl (1S,4S)-2,5-diazabicyclo[2.2.1]heptane-2-carboxylate (72.6 mg, 366 μmol), sodium tert-butoxide (93.8 mg, 976 μmol), XPhos (23.3 mg, 48.8 μmol), Pd₂(dba)₃ (22.3 mg, 24.4 μmol) in 1,4-dioxane (2.5 ml) gave the titled compound (28.0 mg, 16% yield) after flash column chromatography on silica (ethyl acetate/hexane).

LC-MS (LC-MS METHOD 2): Rt=1.72 min; MS (ESIpos): m/z=715 [M+H]⁺

1H NMR (400 MHz, DMSO-d6) δ ppm 8.28-8.39 (m) 7.55 (br t) 7.08-7.33 (m) 5.75-5.86 (m) 4.44-4.50 (m) 4.43 (br s) 3.63-3.71 (m) 3.36-3.51 (m) 2.67 (dt) 2.52-2.58 (m) 2.27-2.35 (m) 1.90-2.00 (m) 1.45-1.62 (m) 1.21-1.44 (m) 0.61-0.85 (m) 0.31-0.53 (m).

### Intermediate 32

### tert-butyl 4-(4-{[(1R)-1-(3-{1,1-difluoro-2-methyl-2-[(triethylsilyl)oxy]propyl}-2-fluorophenyl)ethyl]amino}-2,7-dimethylpyrido[2,3-d]pyrimidin-6-yl)-3,6-dihydropyridine-1(2H)-carboxylate

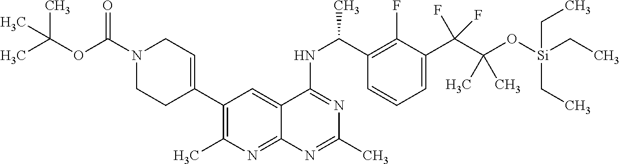 [SMILES: CC[Si](CC)(CC)OC(C)(C)C(F)(F)c1cccc([C@@H](C)Nc2nc(C)nc3nc(C)c(C4=CCN(C(=O)OC(C)(C)C)CC4)cc23)c1F]

Using the method described for Example 21, Intermediate 31 (6-bromo-N-[(1R)-1-(3-{1,1-difluoro-2-methyl-2-[(triethylsilyl)oxy]propyl}-2-fluorophenyl)ethyl]-2,7-dimethylpyrido[2,3-d]pyrimidin-4-amine (200 mg, 335 μmol), tert-butyl 4-(4,4,5,5-tetramethyl-1,3,2-dioxaborolan-2-yl)-3,6-dihydropyridine-1(2H)-carboxylate (114 mg, 368 μmol), bis(triphenylphosphine)-palladium(II) chloride (23 mg, 33 μmol) and potassium carbonate (69.4 mg, 502 μmol) in 1,2-dimethoxyethane (1.7 ml) and ethanol (1.7 ml) gave the titled compound (116 mg, 50% yield) after flash column chromatography on silica(ethyl acetate/hexane).

LC-MS (LC-MS METHOD 2): Rₜ=1.76 min; MS (ESIpos): m/z=701 [M+H]⁺

¹H NMR (400 MHz, DMSO-d₆) δ ppm 8.51-8.60 (m) 7.52-7.65 (m) 7.25-7.30 (m) 7.21 (t) 5.75-5.83 (m) 4.00-4.06 (m) 3.61 (br t) 2.52-2.68 (m) 2.40 (br s) 2.31-2.36 (m) 1.56 (d) 1.45 (s) 1.33 (br d) 0.65-0.73 (m) 0.38-0.48 (m).

### Intermediate 33

### tert-butyl 6-[4-({(1R)-1-[3-(difluoromethyl)-2-fluorophenyl]ethyl}amino)-2-methyl-7-(trifluoromethyl)pyrido[2,3-d]pyrimidin-6-yl]-2,6-diazaspiro[3.3]heptane-2-carboxylate

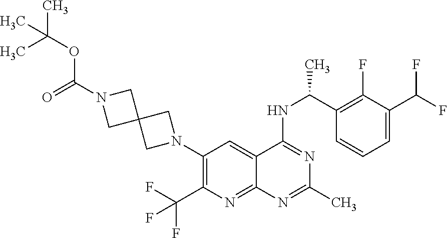 [SMILES: Cc1nc(N[C@H](C)c2cccc(C(F)F)c2F)c2cc(N3CC4(CN(C(=O)OC(C)(C)C)C4)C3)c(C(F)(F)F)nc2n1]

Using the method described for Example 2, intermediate 33 (6-bromo-N-{(1R)-1-[3-(difluoromethyl)-2-fluorophenyl]ethyl}-2-methyl-7-(trifluoromethyl)pyrido[2,3-d]pyrimidin-4-amine (100 mg, 209 μmol), tert-butyl 2,6-diazaspiro[3.3]heptane-2-carboxylate (45.5 mg, 230 μmol), caesium carbonate (81.6 mg, 250 μmol), XPhos (7.96 mg, 16.7 μmol), palladium(II) acetate (2.34 mg, 10.4 μmol) in toluene (2.7 ml) gave the titled compound (40.0 mg, 32% yield) after flash column chromatography on silica u (ethyl acetate/hexane).

LC-MS (LC-MS METHOD 2): Rₜ=1.44 min; MS (ESIpos): m/z=598 [M+H]⁺

### Intermediate 34

### tert-butyl 6-[4-{[(1R)-1-(3-{1,1-difluoro-2-methyl-2-[(triethylsilyl)oxy]propyl}-2-fluorophenyl)ethyl]amino}-2-methyl-7-(trifluoromethyl)pyrido[2,3-d]pyrimidin-6-yl]-2,6-diazaspiro[3.3]heptane-2-carboxylate

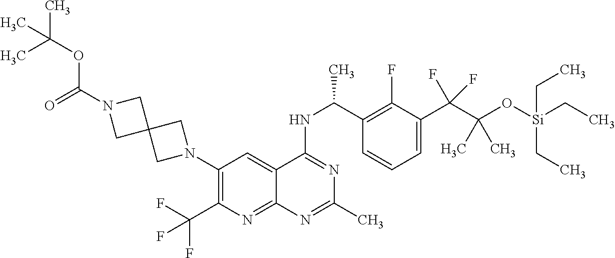 [SMILES: CC[Si](CC)(CC)OC(C)(C)C(F)(F)c1cccc([C@@H](C)Nc2nc(C)nc3nc(C(F)(F)F)c(N4CC5(CN(C(=O)OC(C)(C)C)C5)C4)cc23)c1F]

Using the method described for Example 2, Intermediate 34 (6-bromo-N-[(1R)-1-(3-{1,1-difluoro-2-methyl-2-[(triethylsilyl)oxy]propyl}-2-fluorophenyl)ethyl]-2-methyl-7-(trifluoromethyl)pyrido[2,3-d]pyrimidin-4-amine (70.0 mg, 107 μmol), tert-butyl 2,6-diazaspiro[3.3]heptane-2-carboxylate (23.4 mg, 118 μmol), caesium carbonate (42.0 mg, 129 μmol), XPhos (4.10 mg, 8.59 μmol), palladium(II) acetate (1.21 mg, 5.37 μmol) in toluene (1.4 ml) gave the titled compound (80 mg), which was used directly for the subsequent step.

### Intermediate 35

### tert-butyl 6-[2-methyl-4-({(1R)-1-[2-methyl-3-(trifluoromethyl)phenyl]ethyl}amino)-7-(trifluoromethyl)pyrido[2,3-d]pyrimidin-6-yl]-2,6-diazaspiro[3.3]heptane-2-carboxylate

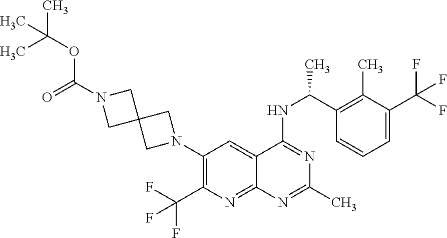 [SMILES: Cc1nc(N[C@H](C)c2cccc(C(F)(F)F)c2C)c2cc(N3CC4(CN(C(=O)OC(C)(C)C)C4)C3)c(C(F)(F)F)nc2n1]

Using the method described for Example 2, Intermediate 35 (6-bromo-2-methyl-N-{(1R)-1-[2-methyl-3-(trifluoromethyl)phenyl]ethyl}-7-(trifluoromethyl)pyrido[2,3-d]pyrimidin-4-amine (60.0 mg, 122 μmol), tert-butyl 2,6-diazaspiro[3.3]heptane-2-carboxylate (36.2 mg, 182 μmol), caesium carbonate (63.4 mg, 195 μmol), XPhos (4.64 mg, 9.73 μmol), palladium(II) acetate (1.37 mg, 6.08 μmol) in toluene (1.6 ml) gave the titled compound (20.0 mg, 27% yield) after preparative HPLC (basic method).

LC-MS (LC-MS METHOD 2): Rₜ=1.53 min; MS (ESIpos): m/z=612 [M+H]⁺

¹H-NMR (400 MHz, DMSO-d6) δ[ppm]: 1.390 (16.00), 1.577 (1.66), 1.594 (1.65), 2.323 (0.97), 2.328 (6.13), 2.518 (3.37), 2.523 (2.45), 2.608 (2.12), 2.665 (0.67), 2.669 (0.96), 2.673 (0.68), 4.080 (1.35), 4.227 (1.96), 7.367 (0.54), 7.546 (0.59), 7.565 (0.47), 7.738 (0.51), 7.757 (0.46), 7.970 (1.18), 8.891 (0.41).

### Intermediate 36

### 2-amino-5-bromo-6-methoxynicotinamide

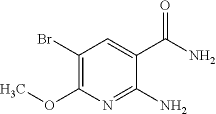

To a solution of 2-amino-6-methoxynicotinamide (CAS 1298123-77-9, 30 g, 175 mmol) in N,N-dimethylformamide was added N-bromosuccinimide at 25° C., the reaction mixture was stirred at 25° C. for 2 hours. The mixture was concentrated, and the residue was washed with water (200 ml) and dried under reduced pressure to give the titled compound (19 g, 65%) as a brown solid.

LC-MS (LC-MS METHOD 3): Rₜ=0.80 min; MS (ESIpos): m/z=244.0/246.0 [M+H]⁺

### Intermediate 37

### 6-bromo-7-methoxy-2-methylpyrido[2,3-d]pyrimidin-4-ol

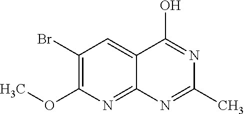

A mixture of Intermediate 36 (5 g, 20.3 mmol), 2-methoxyethanol (3 ml) and 1,1,1-triethoxyethane (50 ml) was stirred at 120° C. for 2 hours. The mixture was cooled to room temperature and the resulting suspension was filtered. The filter cake was washed with methanol to give the titled compound (2.9 g, 53%) as a brown solid.

LC-MS (LC-MS METHOD 3): Rₜ=0.67 min; MS (ESIpos): m/z=269.8/271.8 [M+H]⁺

### Intermediate 38

### 6-bromo-7-methoxy-2-methyl-N-{(1R)-1-[2-methyl-3-(trifluoromethyl)phenyl]ethyl}pyrido[2,3-d]pyrimidin-4-amine

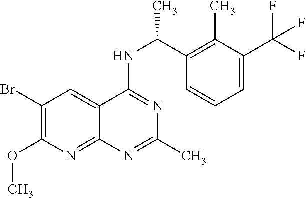 [SMILES: COc1nc2nc(C)nc(N[C@H](C)c3cccc(C(F)(F)F)c3C)c2cc1Br]

To a solution of 6-bromo-7-methoxy-2-methylpyrido[2,3-d]pyrimidin-4-ol (Intermediate 37, 2.5 g, 9.3 mmol), (1R)-1-[2-methyl-3-(trifluoromethyl)phenyl]ethan-1-amine (1.2 g, 11.1 mmol) and pyBOP (6.3 g, 12.0 mmol) in DMF (47 mL) was added DBU (5.5 mL, 37.0 mmol) and the reaction mixture was stirred at room temperature overnight. The mixture was diluted with water and extracted with dichloromethane. The organic phase was washed with water, brine, dried over Na₂SO₄, filtered, and concentrated under reduced pressure. The titled compound (4.2 g, 99% yield) was obtained after purification by flash column chromatography (hexane/ethyl acetate).

LC-MS (LC-MS METHOD 2): Rₜ=0.67 min; MS (ESIpos): m/z=453.4/455.4 [M+H]⁺

¹H NMR (400 MHz, DMSO-d₆) δ ppm 9.10 (s) 8.49-8.65 (m) 8.42-8.47 (m) 7.86 (d) 7.74 (d) 7.43-7.62 (m) 7.32-7.42 (m) 5.65 (quin) 4.32 (q) 3.99-4.08 (m) 3.99 (s) 3.70-3.83 (m) 3.48-3.69 (m) 3.05-3.31 (m) 2.57-2.61 (m) 2.36-2.48 (m) 2.32 (s) 1.89-1.96 (m) 1.66-1.88 (m) 1.52 (d) 1.14-1.43 (m).

### Intermediate 39

### 6-bromo-2,7-dimethylpyrido[2,3-d]pyrimidin-4-ol

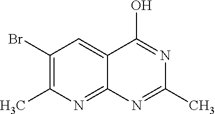

### Step 1: 2-amino-5-bromo-6-methylnicotinic acid

To a solution of methyl-2-amino-5-bromo-6-methylnicotinate trifluoroacetate (15.1 g, 92% purity, 38.7 mmol) in methanol (77 ml) was added aqueous sodium hydroxide (5%, 147 ml, 193 mmol) and the mixture was stirred at room temperature overnight. Methanol was then removed under reduced pressure and the residue was titurated with 2.5 M hydrochloric acid until pH was adjusted to pH 7-8. The pH was then adjusted to pH 4-5 with acetic acid and the resulting precipitate was filtered, washed with water and tert-butyl methyl ether and dried in vacuum at 70° C.. The crude product was directly used in the following reaction.

### Step 2: 6-bromo-2,7-dimethylpyrido[2,3-d]pyrimidin-4-ol

To a solution of the crude product from the previous reaction and ethanimidamide hydrochloride (1:1) (11 g, 116 mmol) in sulfolan (20 ml) and 2-methoxyethanol (80 ml) was added sodium acetate (8.5 g, 104 mmol) and the mixture was stirred for three days at 140° C. 2-Methoxyethanol was removed under reduced pressure and further sulfolan (80 ml), ethanimidamide hydrochloride (1:1) (8.7 g, 93 mmol), and sodium acetate (6.6 g, 81 mmol) were added and the mixture was stirred at 140° C. overnight and at 160° C. for 1 day and at 175° C. for 2 days. After cooling to room temperature, the mixture was poured on ice water (1.5 I) and the resulting precipitate was filtered, washed with water and dried at 70° C. in a vacuum oven overnight, to yield 3.1 g (51%) of the titled compound. The filtrate of the reaction mixture was concentrated under reduced pressure until further solid precipitated. The solid was filtered, washed and dried as described above to yield another batch of the desired product (1.78 g, 30%).

LC-MS (LC-MS METHOD 1): Rₜ=0.71 min; MS (ESIpos): m/z=254.0/256.0 [M+H]⁺

¹H NMR (400 MHz, DMSO-d₆) δ ppm 12.59 (br s) 8.48 (s) 2.68 (s) 2.52-2.58 (m) 2.38 (s)

### Intermediate 40

### 6-bromo-N-[(1R)-1-(3-{1,1-difluoro-2-methyl-2-[(triethylsilyl)oxy]propyl}-2-fluorophenyl)ethyl]-2,7-dimethylpyrido[2,3-d]pyrimidin-4-amine

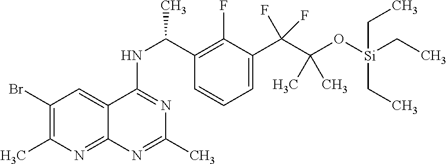 [SMILES: CC[Si](CC)(CC)OC(C)(C)C(F)(F)c1cccc([C@@H](C)Nc2nc(C)nc3nc(C)c(Br)cc23)c1F]

To a solution of 6-bromo-7-methoxy-2-methylpyrido[2,3-d]pyrimidin-4-ol (Intermediate 37, 2.5 g, 9.3 mmol), (1R)-1-(3-{1,1-difluoro-2-methyl-2-[(triethylsilyl)oxy]propyl}-2-fluorophenyl)ethan-1-amine (1.14 g, 3.1 mmol) and pyBOP (1.4 g, 2.7 mmol) in DMF (16 mL) was added DBU (1.25 mL, 8.4 mmol) and the reaction mixture was stirred at room temperature overnight. The mixture was diluted with water and extracted with dichloromethane. The organic phase was washed with water, brine, dried over Na₂SO₄, filtered, and concentrated under reduced pressure. The titled compound (784 mg, 63% yield) was obtained after purification by flash column chromatography (hexane/ethyl acetate).

LC-MS (LC-MS METHOD 2): Rₜ=1.76 min; MS (ESIpos): m/z=597.5/599.5 [M+H]⁺

¹H NMR (400 MHz, DMSO-d₆) δ ppm 9.15 (s) 8.72 (d) 7.67-7.84 (m) 7.61 (br t) 7.36-7.47 (m) 7.26-7.31 (m) 7.21 (t) 5.76 (quin) 4.62 (q) 2.68-2.69 (m) 2.52-2.52 (m) 2.33-2.35 (m) 1.56 (d) 1.46 (d) 1.28-1.37 (m) 0.76-0.83 (m) 0.66-0.71 (m) 0.39-0.54 (m).

### Intermediate 41

### 6-bromo-N-{(1R)-1-[2-methyl-3-(trifluoromethyl)phenyl]ethyl}pyrido[2,3-d]pyrimidin-4-amine

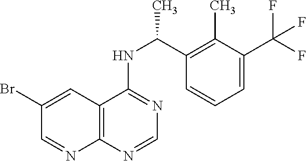 [SMILES: Cc1c([C@@H](C)Nc2ncnc3ncc(Br)cc23)cccc1C(F)(F)F]

To a suspension of 6-bromo-4-chloropyrido[2,3-d]pyrimidine (970 mg, 3.97 mmol) in DMA (6 mL) was added Et₃N (1.1 mL, 7.9 mmol), followed by (1R)-1-[2-methyl-3-(trifluoromethyl)phenyl]ethan-1-amine (887 mg, 4.36 mmol). The resulting reaction mixture was stirred overnight at RT. The reaction mixture was subsequently diluted with H₂O and extracted with ethyl acetate. The organic phase was washed with brine, filtered through a hydrophobic filter, and concentrated under reduced pressure. The titled compound (1.46 g, 95% purity, 85% yield) was isolated after flash column chromatography (hexane/ethyl acetate).

LC-MS (LC-MS METHOD 2): Rt=1.25 min; MS (ESIpos): m/z=411.3, 413.1 [M+H]⁺

¹H NMR (DMSO-d6) δ: 9.27 (d, 1H), 9.06 (d, 1H), 8.94 (d, 1H), 8.58 (s, 1H), 7.77 (d, 1H), 7.57 (d, 1H), 7.37 (m, 1H), 5.73 (m, 1H), 2.52-2.56 (m, 3H), 1.56 (d, 3H)

### Intermediate 42

### 6-bromo-N-{(1R)-1-[3-(difluoromethyl)-2-fluorophenyl]ethyl}-2-methylpyrido[2,3-d]pyrimidin-4-amine

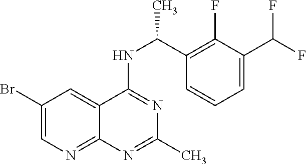 [SMILES: Cc1nc(N[C@H](C)c2cccc(C(F)F)c2F)c2cc(Br)cnc2n1]

To a solution of 6-bromo-2-methylpyrido[2,3-d]pyrimidin-4-ol (Intermediate 1, 200 mg, 833 μmol), (1R)-1-[2-methyl-3-(difluoromethyl)-2-fluorophenyl]ethanamine hydrochloride (226 mg, 1.0 mmol) and pyBOP (564 mg, 1.1 mmol) in DMF (6 mL) was added DBU (497 μl, 3.3 mmol) and the reaction mixture was stirred at RT overnight. The mixture was diluted with water and extracted with ethyl acetate. The organic phase was dried over Na₂SO₄ and concentrated under reduced pressure. The titled compound (267 mg, 78%) was obtained after purification by flash column chromatography (ethyl acetate/hexane).

LC-MS (LC-MS METHOD 2): Rₜ=1.17 min; MS (ESIpos): m/z=411.2/413.2 [M+H]⁺

1H NMR (DMSO-d6) δ: 9.19 (d, 4H), 9.00 (d, 4H), 8.82 (d, 4H), 7.69 (br m, 4H), 7.51 (br m, 4H), 7.37 (s, 1H), 7.23-7.33 (m, 6H), 7.10 (s, 1H), 5.75 (m, 4H), 4.03 (m, 1H), 3.37-3.43 (m, 3H), 3.21-3.31 (m, 2H), 2.38 (s, 13H), 1.99 (s, 2H), 1.60 (d, 12H), 1.15-1.24 (m, 2H)

### EXPERIMENTAL—EXAMPLES

### Example 1

### 6-bromo-N-{(1R)-1-[3-(trifluoromethyl)phenyl]ethyl}pyrido[2,3-d]pyrimidin-4-amine

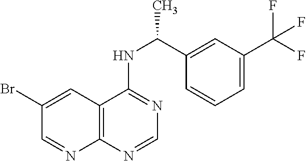 [SMILES: C[C@@H](Nc1ncnc2ncc(Br)cc12)c1cccc(C(F)(F)F)c1]

To a suspension of 6-bromo-4-chloropyrido[2,3-d]pyrimidine (200 mg, 818 μmol) in 2 ml DMA was added Et₃N (230 μl, 1.6 mmol), followed by (1R)-1-[3-(trifluoromethyl)phenyl]ethan-1-amine (170 mg, 900 μmol). The resulting reaction mixture was stirred overnight at RT. The reaction mixture was subsequently diluted with H₂O and extracted with ethyl acetate. The organic phase was washed with H₂O, filtered through a hydrophobic filter, and concentrated under reduced pressure. The titled compound (160 mg, 47%) was isolated after silica chromatography (hexane/ethyl acetate).

LC-MS (LC-MS METHOD 2): Rₜ=1.18 min; MS (ESIpos): m/z=397.3, 399.3 [M+H]⁺

¹H-NMR (400 MHz, DMSO-d6) δ[ppm]: 1.595 (14.95), 1.613 (14.89), 1.987 (0.52), 2.518 (3.53), 2.523 (2.31), 5.576 (0.47), 5.593 (1.98), 5.611 (3.01), 5.628 (1.94), 5.646 (0.43), 7.544 (1.32), 7.563 (3.94), 7.582 (4.05), 7.596 (4.48), 7.616 (1.63), 7.742 (3.30), 7.760 (2.62), 7.798 (5.26), 8.579 (16.00), 8.896 (2.87), 8.914 (2.77), 9.065 (10.03), 9.071 (11.73), 9.230 (9.87), 9.236 (9.10).

### Example 2

### N-{(3R)-1-[4-({(1R)-1-[3-(trifluoromethyl)phenyl]ethyl}amino)pyrido[2,3-d]pyrimidin-6-yl]pyrrolidin-3-yl}acetamide

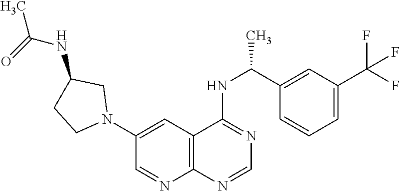 [SMILES: CC(=O)N[C@@H]1CCN(c2cnc3ncnc(N[C@H](C)c4cccc(C(F)(F)F)c4)c3c2)C1]

To a suspension of Example 1 (50.0 mg, 126 μmol) and N-[(3R)-pyrrolidin-3-yl]acetamide (48.4 mg, 378 μmol) in toluene (1 mL) was added Pd₂dba₃ (11.5 mg, 12.6 μmol), 2-(dicyclohexylphosphino)-2′,4′,6′-tri-1-propyl-1,1′-biphenyl (18 mg, 37.8 μmol) and caesium carbonate (164 mg, 504 μmol) under argon. The reaction mixture was stirred at 105° C. overnight. The mixture was filtered through a hydrophobic filter, H₂O was added, and the aqueous phase was extracted with ethyl acetate. The org. phase was concentrated under reduced pressure. The titled compound (10 mg, 17%) was obtained after purification by preparative HPLC (basic method).

LC-MS (LC-MS METHOD 2): Rₜ=1.01 min; MS (ESIpos): m/z=445.5 [M+H]⁺

¹H-NMR (400 MHz, DMSO-d6) δ[ppm]: 1.610 (5.57), 1.627 (5.56), 1.830 (16.00), 1.951 (0.47), 1.968 (0.75), 1.981 (0.81), 1.998 (0.58), 2.216 (0.64), 2.233 (0.77), 2.249 (0.70), 2.264 (0.54), 3.228 (0.94), 3.238 (0.99), 3.254 (1.14), 3.263 (1.16), 3.451 (0.43), 3.475 (0.83), 3.488 (0.90), 3.509 (0.53), 3.558 (0.51), 3.576 (1.12), 3.599 (0.83), 3.641 (0.94), 3.657 (1.10), 3.666 (1.01), 3.682 (0.86), 4.433 (0.82), 4.446 (0.79), 5.632 (0.84), 5.650 (1.26), 5.668 (0.84), 7.544 (0.51), 7.563 (1.69), 7.581 (3.31), 7.605 (0.71), 7.621 (2.21), 7.627 (2.16), 7.721 (1.52), 7.740 (1.24), 7.763 (2.68), 8.215 (1.43), 8.232 (1.39), 8.278 (5.92), 8.415 (1.44), 8.434 (1.39), 8.569 (2.71), 8.575 (2.63).

### Example 3

### 6-(4-methylpiperazin-1-yl)—N-{(1R)-1-[3-(trifluoromethyl)phenyl]ethyl}pyrido[2,3-d]pyrimidin-4-amine

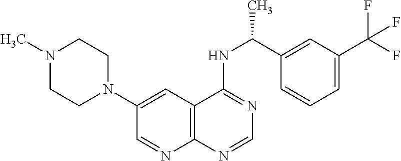 [SMILES: C[C@@H](Nc1ncnc2ncc(N3CCN(C)CC3)cc12)c1cccc(C(F)(F)F)c1]

Using the method described for Example 2: Example 1 (50 mg, 126 μmol) and 1-methylpiperazine (25.2 mg, 252 μmol) gave the titled compound (10 mg, 18%) after purification by preparative HPLC (acidic method).

LC-MS (LC-MS METHOD 2): Rₜ=1.06 min; MS (ESIpos): m/z=417.5 [M+H]⁺

¹H-NMR (400 MHz, DMSO-d6) δ[ppm]: 1.613 (7.03), 1.631 (7.07), 2.262 (16.00), 2.336 (0.75), 2.453 (0.46), 2.518 (12.36), 2.523 (10.80), 2.534 (5.57), 2.546 (3.96), 2.678 (0.78), 3.344 (4.53), 3.358 (4.75), 3.370 (3.51), 5.627 (0.98), 5.644 (1.45), 5.662 (0.95), 7.544 (0.63), 7.564 (1.97), 7.582 (2.70), 7.589 (2.72), 7.609 (0.69), 7.725 (1.71), 7.743 (1.33), 7.766 (2.81), 8.025 (2.60), 8.033 (2.59), 8.352 (7.93), 8.503 (1.57), 8.522 (1.53), 8.925 (3.41), 8.933 (3.28).

### Example 4

### 1-{4-[4-({(1R)-1-[3-(trifluoromethyl)phenyl]ethyl}amino)pyrido[2,3-d]pyrimidin-6-yl]piperazin-1-yl}ethan-1-one

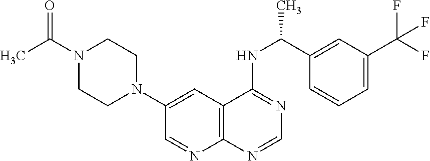 [SMILES: CC(=O)N1CCN(c2cnc3ncnc(N[C@H](C)c4cccc(C(F)(F)F)c4)c3c2)CC1]

Using the method described for Example 2: Example 1 (50 mg, 126 μmol) and 1-(piperazin-1-yl)ethan-1-one (32.3 mg, 252 μmol) gave the titled compound (6.0 mg, 10%) after purification by preparative HPLC (basic method).

LC-MS (LC-MS METHOD 2): Rₜ=1.01 min; MS (ESIpos): m/z=445.5 [M+H]⁺

¹H-NMR (400 MHz, DMSO-d6) δ[ppm]: 1.230 (0.46), 1.627 (5.18), 1.644 (5.16), 2.075 (16.00), 2.523 (4.31), 2.673 (1.13), 3.397 (2.59), 3.410 (2.80), 3.422 (2.24), 3.651 (3.20), 3.664 (3.28), 5.627 (0.74), 5.645 (1.14), 5.663 (0.75), 7.540 (0.52), 7.559 (1.52), 7.578 (2.34), 7.584 (2.27), 7.604 (0.58), 7.754 (1.31), 7.772 (1.08), 7.792 (2.25), 8.185 (1.47), 8.363 (6.27), 8.711 (0.73), 8.728 (0.76), 8.941 (2.53), 8.948 (2.45).

### Example 5

### 1-{4-[4-({(1R)-1-[3-(trifluoromethyl)phenyl]ethyl}amino)pyrido[2,3-d]pyrimidin-6-yl]-3,6-dihydropyridin-1(2H)-yl}ethan-1-one

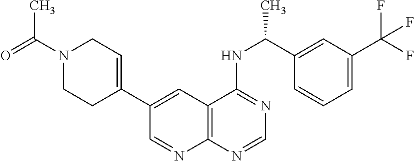 [SMILES: CC(=O)N1CC=C(c2cnc3ncnc(N[C@H](C)c4cccc(C(F)(F)F)c4)c3c2)CC1]

To a solution of Example 1 (110 mg, 277 μmol) and 1-[4-(4,4,5,5-tetramethyl-1,3,2-dioxaborolan-2-yl)-3,6-dihydropyridin-1(2H)-yl]ethan-1-one (188 mg, 748 μmol) in 1,4-dioxane (3.2 mL) was added K₃PO₄ solution (830 μl, 0.50 M, 420 μmol) and XPhosPdG2 (32.7 mg, 41.5 μmol) under argon. The reaction mixture was stirred at 100° C. overnight. The mixture was diluted with CH₂Cl₂, the org. phase was filtered through a hydrophobic filter and concentrated under reduced pressure. The titled compound (73.7 mg, 57%) was obtained after purification by preperative HPLC (basic method).

LC-MS (LC-MS METHOD 2): Rₜ=1.06 min; MS (ESIpos): m/z=442.5 [M+H]⁺

¹H-NMR (400 MHz, DMSO-d6) δ[ppm]: 1.626 (9.99), 1.644 (9.90), 2.069 (12.40), 2.108 (16.00), 2.185 (0.48), 2.327 (0.53), 2.518 (1.96), 2.523 (1.30), 2.624 (0.84), 2.665 (0.59), 2.669 (0.74), 2.673 (0.57), 2.708 (1.19), 3.697 (1.65), 3.711 (3.52), 3.725 (2.39), 3.738 (2.48), 3.752 (1.16), 4.185 (2.42), 4.192 (2.43), 4.232 (1.98), 4.240 (1.95), 5.648 (1.40), 5.666 (2.09), 5.684 (1.33), 6.502 (2.51), 6.506 (2.36), 7.550 (0.95), 7.570 (2.90), 7.588 (3.44), 7.598 (3.69), 7.618 (1.12), 7.743 (2.50), 7.761 (1.98), 7.785 (4.09), 8.517 (12.56), 8.835 (3.87), 8.840 (4.30), 8.860 (1.23), 9.154 (2.87), 9.160 (2.85), 9.184 (2.31), 9.190 (2.20).

### Example 6

### 6-bromo-2-methyl-N-{(1R)-1-[2-methyl-3-(trifluoromethyl)phenyl]ethyl}pyrido[2,3-d]pyrimidin-4-amine

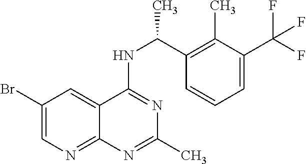 [SMILES: Cc1nc(N[C@H](C)c2cccc(C(F)(F)F)c2C)c2cc(Br)cnc2n1]

To a solution of 6-bromo-2-methylpyrido[2,3-d]pyrimidin-4-ol (Intermediate 1, 140 mg, 583 μmol), (1R)-1-[2-methyl-3-(trifluoromethyl)phenyl]ethan-1-amine (142 mg, 700 μmol) and pyBOP (395 mg, 758 μmol) in DMF (4.5 mL) was added DBU (350 μl, 2.3 mmol) and the reaction mixture was stirred at RT overnight. The mixture was diluted with water and extracted with CH₂Cl₂. The org. phase was dried over Na₂SO₄ and concentrated under reduced pressure. The titled compound (76.0 mg, 31%) was obtained after purification by silica chromatography and subsequent recrystallization from CH₂Cl₂/Et₂O.

LC-MS (LC-MS METHOD 2): Rₜ=1.32 min; MS (ESIpos): m/z=425.3 [M+H]⁺

1H-NMR (400 MHz, DMSO-d6) δ[ppm]: 1.543 (4.61), 1.560 (4.53), 2.375 (16.00), 2.518 (1.54), 2.523 (1.00), 2.610 (5.18), 5.675 (0.68), 5.692 (1.04), 5.710 (0.66), 7.341 (0.57), 7.361 (1.28), 7.381 (0.74), 7.543 (1.42), 7.561 (1.13), 7.751 (1.23), 7.770 (1.10), 8.903 (0.94), 8.920 (0.91), 8.982 (3.32), 8.988 (3.53), 9.176 (3.04), 9.182 (2.80).

### Example 7

### tert-butyl 6-[2-methyl-4-({(1R)-1-[2-methyl-3-(trifluoromethyl)phenyl]ethyl}amino)pyrido[2,3-d]pyrimidin-6-yl]-2,6-diazaspiro[3.3]heptane-2-carboxylate

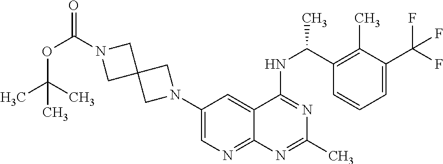 [SMILES: Cc1nc(N[C@H](C)c2cccc(C(F)(F)F)c2C)c2cc(N3CC4(CN(C(=O)OC(C)(C)C)C4)C3)cnc2n1]

To a solution of Example 6 (45 mg, 106 μmol), tert-butyl 2,6-diazaspiro[3.3]heptane-2-carboxylate (42 mg, 212 μmol) and XPhos (10.1 mg, 21.2 μmol) in 1,4-dioxane (0.6 mL) was added NaOtBu (22.4 mg, 233 μmol) under argon, followed by Pd₂dba₃ (8.33 mg, 10.6 μmol). The reaction mixture was stirred at 100° C. for 2 h. The mixture was filtered through a syringe filter and concentrated under reduced pressure. The titled compound (26.2 mg, 43%) was obtained after purification by preparative HPLC (basic method).

LC-MS (LC-MS METHOD 2): Rₜ=1.39 min; MS (ESIpos): m/z=543.8 [M+H]⁺

1H-NMR (400 MHz, DMSO-d6) δ[ppm]: 1.390 (16.00), 1.546 (1.91), 1.563 (1.89), 2.305 (5.45), 2.327 (0.40), 2.522 (1.13), 2.615 (2.48), 4.073 (1.69), 4.107 (0.42), 4.128 (3.30), 5.694 (0.46), 7.348 (0.62), 7.523 (0.66), 7.542 (0.54), 7.661 (0.87), 7.669 (0.87), 7.736 (0.60), 7.756 (0.54), 8.316 (1.19), 8.323 (1.15), 8.485 (0.50), 8.503 (0.48).

### Example 8

### 1-{6-[2-methyl-4-({(1R)-1-[2-methyl-3-(trifluoromethyl)phenyl]ethyl}amino)pyrido[2,3-d]pyrimidin-6-yl]-2,6-diazaspiro[3.3]heptan-2-yl}ethan-1-one

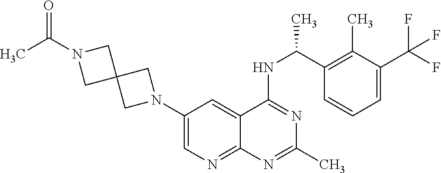 [SMILES: CC(=O)N1CC2(C1)CN(c1cnc3nc(C)nc(N[C@H](C)c4cccc(C(F)(F)F)c4C)c3c1)C2]

To a solution of Example 7 (21.0 mg, 38.7 μmol) in CH₂Cl₂ (0.2 mL) was added Et₃SiH (0.62 μl, 3.9 μmol) followed by trifluoroacetic acid (38 μl) at 0° C. The mixture was stirred at room temperature for 6 h. Toluene (1 mL) was added, and the mixture was concentrated under reduced pressure. The residue was dissolved in CH₂Cl₂ (0.2 mL), DIPEA (14.5 μl, 85.1 μmol) and Ac₂O (4.02 μl, 42.6 μmol) were added and the reaction mixture was stirred at RT for 1 h. Toluene (1 mL) was added, and the mixture was concentrated under reduced pressure. The titled compound (14.0 mg, 67%) was obtained after purification by preparative TLC (CH₂Cl₂/EtOH 9:1).

LC-MS (LC-MS METHOD 2): Rₜ=1.16 min; MS (ESIpos): m/z=485.5 [M+H]⁺

1H-NMR (400 MHz, DMSO-d6) δ[ppm]: 1.222 (0.72), 1.240 (3.66), 1.255 (4.29), 1.271 (2.32), 1.557 (4.84), 1.575 (4.88), 1.767 (16.00), 1.907 (1.05), 2.330 (12.93), 2.518 (4.29), 2.523 (2.86), 2.612 (6.19), 2.660 (0.42), 2.665 (0.88), 2.669 (1.22), 2.673 (0.84), 4.061 (4.76), 4.131 (0.76), 4.156 (7.28), 4.181 (0.76), 4.339 (4.93), 5.699 (0.72), 5.716 (1.09), 5.734 (0.67), 7.338 (0.72), 7.358 (1.52), 7.377 (0.88), 7.534 (1.68), 7.552 (1.35), 7.702 (1.98), 7.709 (1.98), 7.744 (1.52), 7.763 (1.35), 8.347 (3.28), 8.354 (3.28).

### Example 9

### 1-{4-[4-({(1R)-1-[3-(trifluoromethyl)phenyl]ethyl}amino)pyrido[2,3-d]pyrimidin-6-yl]piperidin-1-yl}ethan-1-one

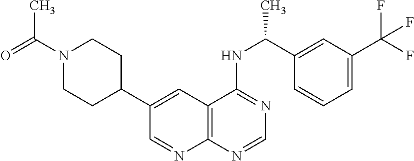 [SMILES: CC(=O)N1CCC(c2cnc3ncnc(N[C@H](C)c4cccc(C(F)(F)F)c4)c3c2)CC1]

To a solution of 1-{4-[4-({(1R)-1-[3-(trifluoromethyl)phenyl]ethyl}amino)pyrido[2,3-d]pyrimidin-6-yl]-3,6-dihydropyridin-1(2H)-yl}ethan-1-one (Example 5, 65.0 mg, 147 μmol) in MeOH (3 mL) was added Pd/C (10%, 15.7 mg, 14.7 μmol) and the mixture was stirred under H₂ atmosphere at rt for 3 h. The mixture was filtered and concentrated under reduced pressure. Purification by HPLC (acidic method) gave the titled compound (6.4 mg, 9% yield).

LC-MS (LC-MS METHOD 1): Rₜ=0.90 min; MS (ESIpos): m/z=444.7 [M+H]⁺

¹H NMR (DMSO-d6) δ: 8.95 (d, 1H), 8.71-8.79 (m, 2H), 8.51 (s, 1H), 7.72-7.79 (m, 2H), 7.55-7.62 (m, 2H), 5.65 (m, 1H), 4.62 (br d, 1H), 4.00 (br d, 1H), 3.15-3.23 (m, 1H), 3.03 (m, 1H), 2.60-2.68 (m, 1H), 1.88-2.07 (m, 5H), 1.59-1.73 (m, 5H), 1.23 (br s, 1H)

### Example 10

### 1-{4-[4-({(1R)-1-[2-methyl-3-(trifluoromethyl)phenyl]ethyl}amino)pyrido[2,3-d]pyrimidin-6-yl]piperazin-1-yl}ethan-1-one

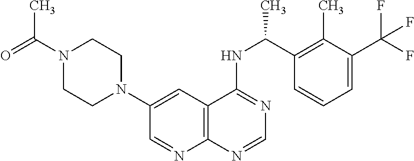 [SMILES: CC(=O)N1CCN(c2cnc3ncnc(N[C@H](C)c4cccc(C(F)(F)F)c4C)c3c2)CC1]

Using the method described for Example 7: Intermediate 41 (6-bromo-N-{(1R)-1-[2-methyl-3-(trifluoromethyl)phenyl]ethyl}pyrido[2,3-d]pyrimidin-4-amine (100 mg, 243 μmol), 1-(piperazin-1-yl)ethan-1-one (93.5 mg, 730 μmol), NaOtBu (46.7 mg, 486 μmol), Pd₂dba₃(11.1 mg, 12.2 μmol), XPhos (11.6 mg, 24.3 μmol) in 1,4-dioxane (2 mL) gave the titled compound (56.2 mg, 95% purity, 48% yield) after purification by HPLC (MeCN/H₂O).

LC-MS (LC-MS METHOD 2): Rₜ=1.08 min; MS (ESIpos): m/z=459.6 [M+H]⁺

¹H NMR (DMSO-d6) δ: 8.95 (d, 1H), 8.56 (d, 1H), 8.37 (s, 1H), 8.10 (d, 1H), 7.76 (d, 1H), 7.57 (d, 1H), 7.37 (m, 1H), 5.76 (m, 1H), 3.66 (m, 4H), 3.35-3.43 (m, 2H), 3.26-3.31 (m, 1H), 2.52-2.56 (m, 4H), 2.08 (s, 3H), 1.57 (d, 3H), 1.23 (br s, 1H)

### Example 11

### N-{(3R)-1-[4-({(1R)-1-[2-methyl-3-(trifluoromethyl)phenyl]ethyl}amino)pyrido[2,3-d]pyrimidin-6-yl]pyrrolidin-3-yl}acetamide

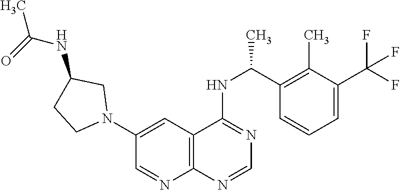 [SMILES: CC(=O)N[C@@H]1CCN(c2cnc3ncnc(N[C@H](C)c4cccc(C(F)(F)F)c4C)c3c2)C1]

Using the method described for Example 7: Intermediate 41 (6-bromo-N-{(1R)-1-[2-methyl-3-(trifluoromethyl)phenyl]ethyl}pyrido[2,3-d]pyrimidin-4-amine (75.0 mg, 182 μmol), N-[(3R)-pyrrolidin-3-yl]acetamide (70.1 mg, 547 μmol), NaOtBu (35.1 mg, 365 μmol), Pd₂dba₃(8.35 mg, 9.12 μmol), XPhos (8.69 mg, 18.2 μmol) in 1,4-dioxane (1.5 mL) gave the titled compound (9.50 mg, 95% purity, 11% yield) after purification by HPLC (basic method).

LC-MS (LC-MS METHOD 2): Rₜ=1.09 min; MS (ESIpos): m/z=459.6 [M+H]⁺

¹H NMR (DMSO-d6) δ: 8.56 (d, 1H), 8.45 (d, 1H), 8.27 (s, 1H), 8.22 (d, 1H), 7.75 (d, 1H), 7.65 (d, 1H), 7.56 (d, 1H), 7.37 (m, 1H), 5.76 (m, 1H), 4.40-4.47 (m, 1H), 3.55-3.69 (m, 2H), 3.36-3.51 (m, 1H), 3.22-3.31 (m, 1H), 2.52-2.56 (m, 3H), 2.18-2.29 (m, 1H), 1.93-2.02 (m, 1H), 1.80-1.86 (m, 3H), 1.57 (d, 3H)

### Example 12

### 1-{4-[4-({(1R)-1-[2-methyl-3-(trifluoromethyl)phenyl]ethyl}amino)pyrido[2,3-d]pyrimidin-6-yl]-3,6-dihydropyridin-1(2H)-yl}ethan-1-one

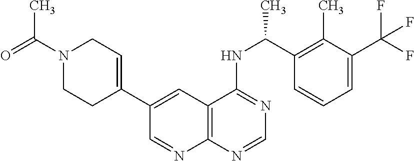 [SMILES: CC(=O)N1CC=C(c2cnc3ncnc(N[C@H](C)c4cccc(C(F)(F)F)c4C)c3c2)CC1]

Using the method described for Example 5: Intermediate 41 (6-bromo-N-{(1R)-1-[2-methyl-3-(trifluoromethyl)phenyl]ethyl}pyrido[2,3-d]pyrimidin-4-amine (100 mg, 243 μmol), 1-[4-(4,4,5,5-tetramethyl-1,3,2-dioxaborolan-2-yl)-3,6-dihydropyridin-1(2H)-yl]ethan-1-one (165 mg, 657 μmol), XPhosPdG2 (28.7 mg, 36.5 μmol), K₃PO₄ solution (730 μL, 0.50 M, 360 μmol) in 1,4-dioxane (2.5 mL) gave the titled compound (41.0 mg, 95% purity, 35% yield) after purification by HPLC (basic method).

LC-MS (LC-MS METHOD 2): Rₜ=1.12 min; MS (ESIpos): m/z=456.6 [M+H]⁺

¹H NMR (DMSO-d6) δ: 9.16 (m, 1H), 8.84-8.89 (m, 2H), 8.51 (s, 1H), 7.77 (d, 1H), 7.58 (d, 1H), 7.38 (m, 1H), 6.50 (br s, 1H), 5.77 (m, 1H), 4.21 (br m, 2H), 3.69-3.76 (m, 2H), 2.58-2.75 (m, 2H), 2.52-2.56 (m, 4H), 2.09 (d, 3H), 1.58 (d, 3H)

### Example 13

### 1-{6-[2-methyl-4-({(1R)-1-[2-methyl-3-(trifluoromethyl)phenyl]ethyl}amino)pyrido[2,3-d]pyrimidin-6-yl]-2,6-diazaspiro[3.3]heptan-2-yl}ethan-1-one-hydrogen chloride (1/1)

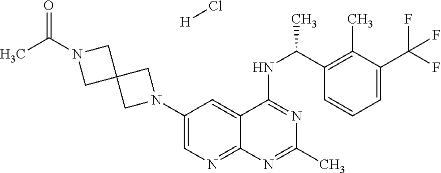 [SMILES: CC(=O)N1CC2(C1)CN(c1cnc3nc(C)nc(N[C@H](C)c4cccc(C(F)(F)F)c4C)c3c1)C2 ; Cl]

To a solution of Example 8 (1-{6-[2-methyl-4-({(1R)-1-[2-methyl-3-(trifluoromethyl)phenyl]ethyl}amino)pyrido[2,3-d]pyrimidin-6-yl]-2,6-diazaspiro[3.3]heptan-2-yl}ethan-1-one (20.5 mg, 42.3 μmol) in dichloromethane (1.0 mL) and MeOH (510 μL) was added HCl/1,4-dioxane (12 μL, 4.0 M, 47 μmol) and the mixture was stirred at RT for 30 min. The mixture was concentrated under reduced pressure and dried at 50° C. to give the titled compound (22.0 mg, 95% purity, 95% yield).

LC-MS (LC-MS METHOD 2): Rₜ=1.13 min; MS (ESIpos): m/z=485 [M+H]⁺

¹H NMR (DMSO-d6) δ: 14.52 (br s, 1H), 10.40 (br s, 1H), 8.41 (d, 1H), 8.14 (d, 1H), 7.90 (d, 1H), 7.60 (d, 1H), 7.42 (m, 1H), 5.88 (m, 1H), 4.35 (s, 2H), 4.18-4.27 (m, 4H), 4.04-4.11 (m, 2H), 3.38-3.60 (m, 1H), 2.59 (s, 3H), 2.52-2.54 (m, 1H), 1.77 (s, 3H), 1.61-1.72 (m, 3H)

### Example 14

### 1-{(1S,4S)-5-[2-methyl-4-({(1R)-1-[2-methyl-3-(trifluoromethyl)phenyl]ethyl}amino)pyrido[2,3-d]pyrimidin-6-yl]-2,5-diazabicyclo[2.2.1]heptan-2-yl}ethan-1-one

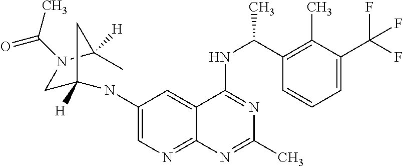

To a solution of Intermediate 2 (tert-butyl (1 S,4S)-5-[2-methyl-4-({(1R)-1-[2-methyl-3-(trifluoromethyl)phenyl]ethyl}amino)pyrido[2,3-d]pyrimidin-6-yl]-2,5-diazabicyclo[2.2.1]heptane-2-carboxylate, 45.2 mg, 83.3 μmol) and Et₃SiH (1.3 μL, 8.3 μmol) in dichloromethane (340 μL) was added TFA (81 μL, 400 μmol) dropwise. The reaction mixture was stirred at RT for 6 h. Toluene (1 mL) was added, and the mixture was concentrated under reduced pressure. The residue was dissolved in dichloromethane (420 μL) and DIPEA (32 μL, 180 μmol) and Ac₂O (8.7 μL, 92 μmol) were added successively at 0° C. The mixture was stirred at RT overnight. Then, toluene (1 mL) was added and the mixture was concentrated under reduced pressure. Purification by HPLC (basic method) gave the titled compound (26.0 mg, 95% purity, 61% yield).

LC-MS (LC-MS METHOD 2): Rₜ=1.15 min; MS (ESIpos): m/z=486 [M+H]⁺

¹H NMR (DMSO-d6) δ: 8.60 (d, 1H), 8.56 (d, 1H), 8.37 (m, 2H), 7.67-7.76 (m, 4H), 7.53 (d, 2H), 7.35 (m, 2H), 5.66-5.74 (m, 2H), 4.89 (s, 1H), 4.84 (s, 1H), 4.76 (br d, 2H), 3.72 (m, 1H), 3.60-3.66 (m, 2H), 3.37-3.45 (m, 2H), 3.20 (d, 1H), 2.61-2.68 (m, 7H), 2.52-2.54 (m, 1H), 2.29-2.34 (m, 6H), 1.93-2.09 (m, 7H), 1.82 (s, 3H), 1.56 (m, 6H)

### Example 15

### 2-methyl-6-(4-methylpiperazin-1-yl)—N-{(1R)-1-[2-methyl-3-(trifluoromethyl)phenyl]ethyl}pyrido[2,3-d]pyrimidin-4-amine

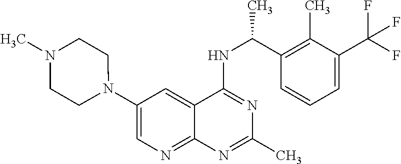 [SMILES: Cc1nc(N[C@H](C)c2cccc(C(F)(F)F)c2C)c2cc(N3CCN(C)CC3)cnc2n1]

Using the method described for Example 7: Example 6 (6-bromo-2-methyl-N-{(1R)-1-[2-methyl-3-(trifluoromethyl)phenyl]ethyl}pyrido[2,3-d]pyrimidin-4-amine, 50.0 mg, 118 μmol), 1-methylpiperazine (35.3 mg, 353 μmol), NaOtBu (45.2 mg, 470 μmol), Pd₂dba₃ (10.8 mg, 11.8 μmol), XPhos (11.2 mg, 23.5 μmol) in 1,4-dioxane (1.2 mL) at 100° C. overnight gave the titled compound (13.8 mg, 95% purity, 25% yield) after purification by HPLC and prep. TLC (dichloromethane/MeOH).

LC-MS (LC-MS METHOD 2): Rₜ=1.20 min; MS (ESIneg): m/z=443 [M−H]⁻

¹H NMR (DMSO-d6) δ: 8.84 (d, 1H), 8.54 (d, 1H), 8.02 (d, 1H), 7.75 (d, 1H), 7.54 (d, 1H), 7.35 (m, 1H), 5.71 (m, 1H), 3.26-3.31 (m, 2H), 2.60-2.68 (m, 3H), 2.52-2.56 (m, 4H), 2.32 (s, 3H), 2.26 (s, 3H), 1.56 (d, 3H)

### Example 16

### N-{(3R)-1-[2-methyl-4-({(1R)-1-[2-methyl-3-(trifluoromethyl)phenyl]ethyl}amino)pyrido[2,3-d]pyrimidin-6-yl]pyrrolidin-3-yl}acetamide

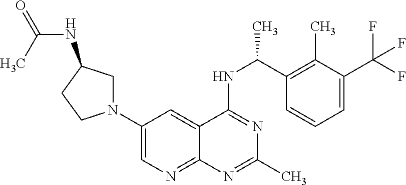 [SMILES: CC(=O)N[C@@H]1CCN(c2cnc3nc(C)nc(N[C@H](C)c4cccc(C(F)(F)F)c4C)c3c2)C1]

Using the method described for Example 7: Example 6 (6-bromo-2-methyl-N-{(1R)-1-[2-methyl-3-(trifluoromethyl)phenyl]ethyl}pyrido[2,3-d]pyrimidin-4-amine, 40.0 mg, 94.1 μmol), N-[(3R)-pyrrolidin-3-yl]acetamide (24.1 mg, 188 μmol), NaOtBu (18.1 mg, 188 μmol), Pd₂dba₃ (4.31 mg, 4.70 μmol), XPhos (4.48 mg, 9.41 μmol) in 1,4-dioxane (890 μL) at 100° C. for 6 h gave the titled compound (6.00 mg, 95% purity, 13% yield) after purification by HPLC (basic method) and prep. TLC (dichloromethane/MeOH 9:1).

LC-MS (LC-MS METHOD 2): Rₜ=1.16 min; MS (ESIpos): m/z=474 [M+H]⁺

¹H NMR (DMSO-d6) δ: 8.49 (d, 1H), 8.44 (d, 1H), 8.21 (d, 1H), 7.75 (d, 1H), 7.62 (d, 1H), 7.53 (d, 1H), 7.36 (m, 1H), 5.72 (m, 1H), 4.39-4.47 (m, 1H), 3.53-3.68 (m, 2H), 3.36-3.49 (m, 1H), 3.20-3.30 (m, 1H), 2.60-2.68 (m, 4H), 2.52-2.54 (m, 3H), 2.19-2.34 (m, 5H), 1.92-2.01 (m, 1H), 1.70-1.88 (m, 4H), 1.56 (d, 3H), 1.06-1.30 (m, 2H), 0.71-0.90 (m, 1H)

### Example 17

### 1-{4-[2-methyl-4-({(1R)-1-[2-methyl-3-(trifluoromethyl)phenyl]ethyl}amino)pyrido[2,3-d]pyrimidine-6-sulfonyl]piperazin-1-yl}ethan-1-one

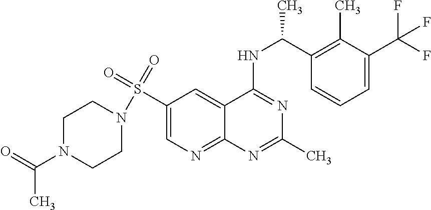 [SMILES: CC(=O)N1CCN(S(=O)(=O)c2cnc3nc(C)nc(N[C@H](C)c4cccc(C(F)(F)F)c4C)c3c2)CC1]

To a solution of 2-methyl-4-({(1R)-1-[2-methyl-3-(trifluoromethyl)phenyl]ethyl}amino)pyrido[2,3-d]pyrimidine-6-sulfonyl chloride (Intermediate 4, 244 mg, 42% purity, 230 μmol) in dichloromethane (2.7 mL) was added 1-(piperazin-1-yl)ethan-1-one (88.6 mg, 691 μmol) and triethylamine (96 μL) and the mixture was stirred at rt for 1 h. The mixture was diluted with dichloromethane and the org. phase was washed with water, sat. NaHCO₃ solution and brine, filtered through a hydrophobic filter and concentrated under reduced pressure. Purification by HPLC (acidic method) gave the titled compound (8.00 mg, 90% purity, 6% yield).

LC-MS (LC-MS METHOD 1): Rₜ=1.10 min; MS (ESIpos): m/z=537.6 [M+H]⁺

¹H NMR (DMSO-d6) δ: 9.39 (d, 1H), 9.35 (d, 1H), 9.11 (d, 1H), 7.76 (d, 1H), 7.56 (d, 1H), 7.37 (m, 1H), 5.69-5.76 (m, 1H), 4.03 (m, 2H), 3.38-3.61 (m, 5H), 2.94-3.08 (m, 4H), 2.52-2.63 (m, 4H), 2.37-2.43 (m, 3H), 1.91-2.02 (m, 6H), 1.59 (d, 3H), 1.15-1.42 (m, 3H)

### Example 18

### N-{(3R)-1-[2,7-dimethyl-4-({(1R)-1-[2-methyl-3-(trifluoromethyl)phenyl]ethyl}amino)pyrido[2,3-d]pyrimidin-6-yl]pyrrolidin-3-yl}acetamide

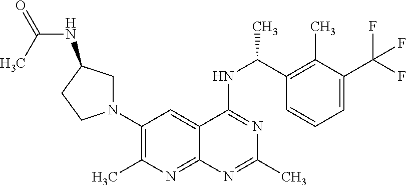 [SMILES: CC(=O)N[C@@H]1CCN(c2cc3c(N[C@H](C)c4cccc(C(F)(F)F)c4C)nc(C)nc3nc2C)C1]

Using the method described for Example 7: 6-bromo-2,7-dimethyl-N-{(1R)-1-[2-methyl-3-(trifluoromethyl)phenyl]ethyl}pyrido[2,3-d]pyrimidin-4-amine (Intermediate 5, 75.0 mg, 171 μmol), N-[(3R)-pyrrolidin-3-yl]acetamide (43.8 mg, 341 μmol), NaOtBu (34.5 mg, 359 μmol), Pd₂dba₃ (15.6 mg, 17.1 μmol), XPhos (16.3 mg, 34.1 μmol) in 1,4-dioxane (890 μL) at 100° C. overnight gave the titled compound (24.0 mg, 95% purity, 27% yield) after purification by HPLC (basic method).

LC-MS (LC-MS METHOD 2): Rₜ=1.17 min; MS (ESIpos): m/z=488 [M+H]⁺

¹H NMR (DMSO-d6) δ: 8.52 (d, 1H), 8.21 (d, 1H), 7.88 (s, 1H), 7.75 (d, 1H), 7.53 (d, 1H), 7.35 (m, 1H), 5.71 (m, 1H), 4.35-4.43 (m, 1H), 3.39-3.53 (m, 2H), 3.22-3.31 (m, 1H), 3.09 (m, 1H), 2.52-2.68 (m, 7H), 2.18-2.34 (m, 4H), 1.82-1.91 (m, 4H), 1.55 (d, 3H)

### Example 19

### 1-{4-[2,7-dimethyl-4-({(1R)-1-[2-methyl-3-(trifluoromethyl)phenyl]ethyl}amino)pyrido[2,3-d]pyrimidin-6-yl]piperazin-1-yl}ethan-1-one

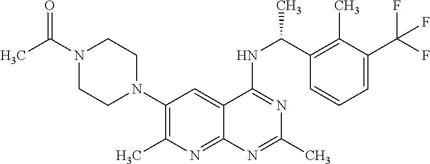 [SMILES: CC(=O)N1CCN(c2cc3c(N[C@H](C)c4cccc(C(F)(F)F)c4C)nc(C)nc3nc2C)CC1]

Using the method described for Example 7: 6-bromo-2,7-dimethyl-N-{(1R)-1-[2-methyl-3-(trifluoromethyl)phenyl]ethyl}pyrido[2,3-d]pyrimidin-4-amine (Intermediate 5, 75.0 mg, 171 μmol), 1-(piperazin-1-yl)ethan-1-one (65.7 mg, 512 μmol), NaOtBu (50.9 mg, 529 μmol), Pd₂dba₃ (7.82 mg, 8.54 μmol), XPhos (8.14 mg, 17.1 μmol) in 1,4-dioxane (1.7 mL) at 100° C. for 24 h gave the titled compound (14.0 mg, 95% purity, 16% yield) after purification by prep. TLC (dichloromethane/EtOH).

LC-MS (LC-MS METHOD 2): Rₜ=1.19 min; MS (ESIpos): m/z=487 [M+H]⁺

¹H NMR (DMSO-d6) δ: 8.56 (d, 1H), 8.30 (s, 1H), 7.75 (d, 1H), 7.54 (d, 1H), 7.36 (m, 1H), 5.71 (m, 1H), 3.62-3.70 (m, 4H), 2.89-3.00 (m, 4H), 2.58-2.63 (m, 6H), 2.52-2.54 (m, 2H), 2.33 (s, 3H), 2.08 (s, 3H), 1.56 (d, 3H)

### Example 20

### 1-{6-[7-methoxy-2-methyl-4-({(1R)-1-[2-methyl-3-(trifluoromethyl)phenyl]ethyl}amino)pyrido[2,3-d]pyrimidin-6-yl]-2,6-diazaspiro[3.3]heptan-2-yl}ethan-1-one

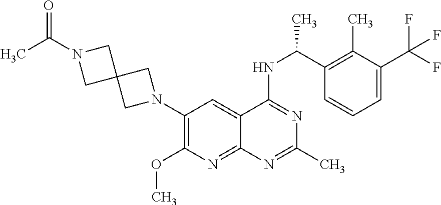 [SMILES: COc1nc2nc(C)nc(N[C@H](C)c3cccc(C(F)(F)F)c3C)c2cc1N1CC2(CN(C(C)=O)C2)C1]

Using the method described for Example 7: 6-bromo-7-methoxy-2-methyl-N-{(1R)-1-[2-methyl-3-(trifluoromethyl)phenyl]ethyl}pyrido[2,3-d]pyrimidin-4-amine (Intermediate 38, 75.0 mg, 165 μmol), oxalic acid 1-(2,6-diazaspiro[3.3]heptan-2-yl)ethan-1-one (½) (45.8 mg, 124 μmol), sodium tert-butoxide (63.3 mg, 659 μmol), Pd₂dba₃ (25.9 mg, 32.9 μmol), XPhos (31.4 mg, 65.9 μmol) in 1,4-dioxane (1.9 mL) at 100° C. overnight gave the titled compound (22.2 mg, 98% purity, 26% yield) after purification by HPLC (basic method).

LC-MS (LC-MS METHOD 2): Rₜ=1.22 min; MS (ESIpos): m/z=515 [M+H]⁺

¹H-NMR (400 MHz, DMSO-d6) δ[ppm]: 1.529 (4.36), 1.546 (4.36), 1.762 (13.67), 1.907 (0.50), 2.279 (14.34), 2.337 (0.59), 2.518 (6.72), 2.523 (4.82), 2.612 (5.46), 2.678 (0.61), 3.926 (16.00), 4.026 (4.29), 4.072 (0.78), 4.095 (3.19), 4.101 (5.90), 4.107 (3.10), 4.130 (0.77), 4.307 (4.40), 5.669 (0.67), 5.687 (1.04), 5.704 (0.67), 7.329 (0.63), 7.349 (1.39), 7.369 (0.81), 7.475 (3.82), 7.519 (1.50), 7.537 (1.21), 7.738 (1.33), 7.758 (1.20), 8.211 (1.14), 8.229 (1.11).

### Example 21

### 1-{4-[7-methoxy-2-methyl-4-({(1R)-1-[2-methyl-3-(trifluoromethyl)phenyl]ethyl}amino)pyrido[2,3-d]pyrimidin-6-yl]-3,6-dihydropyridin-1(2H)-yl}ethan-1-one

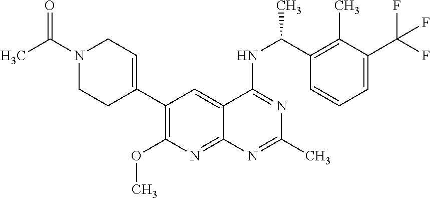 [SMILES: COc1nc2nc(C)nc(N[C@H](C)c3cccc(C(F)(F)F)c3C)c2cc1C1=CCN(C(C)=O)CC1]

A mixture of 6-bromo-7-methoxy-2-methyl-N-{(1R)-1-[2-methyl-3-(trifluoromethyl)phenyl]-ethyl}pyrido[2,3-d]pyrimidin-4-amine (Intermediate 38, 150 mg, 329 μmol), 1-[4-(4,4,5,5-tetramethyl-1,3,2-dioxaborolan-2-yl)-3,6-dihydropyridin-1(2H)-yl]ethan-1-one (99.3 mg, 395 μmol), bis(triphenylphosphine)palladium(II) chloride (23.1 mg, 33 μmol) and potassium carbonate (68.3 mg, 494 μmol) in 1,2-dimethoxyethane (1.4 mL) and ethanol (1.4 mL) was purged with argon and heated in a microwave at 100° C. for 6 h. The mixture was filtered and concentrated under reduced pressure. Purification by HPLC (basic method) and prep. TLC (dichloromethane/EtOH) gave the titled compound (51.9 mg, 95% purity, 30% yield).

LC-MS (LC-MS METHOD 2): Rₜ=1.29 min; MS (ESIpos): m/z=500 [M+H]⁺

1H NMR (DMSO-d6) δ: 8.58 (d, 1H), 8.55 (d, 1H), 7.76 (d, 1H), 7.54 (d, 1H), 7.35 (m, 1H), 6.04-6.09 (m, 1H), 5.70 (m, 1H), 4.11-4.19 (m, 2H), 3.95 (s, 3H), 3.60-3.72 (m, 2H), 2.61 (s, 3H), 2.52-2.58 (m, 2H), 2.40-2.46 (m, 1H), 2.30-2.34 (m, 4H), 2.05-2.10 (m, 3H), 1.53 (d, 3H)

### Example 22

### 1-{4-[7-methoxy-2-methyl-4-({(1R)-1-[2-methyl-3-(trifluoromethyl)phenyl]ethyl}amino)pyrido[2,3-d]pyrimidin-6-yl]piperidin-1-yl}ethan-1-one

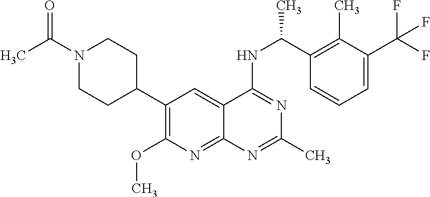 [SMILES: COc1nc2nc(C)nc(N[C@H](C)c3cccc(C(F)(F)F)c3C)c2cc1C1CCN(C(C)=O)CC1]

To a solution of 1-{4-[7-methoxy-2-methyl-4-({(1R)-1-[2-methyl-3-(trifluoromethyl)-phenyl]ethyl}amino)pyrido[2,3-d]pyrimidin-6-yl]-3,6-dihydropyridin-1(2H)-yl}ethan-1-one (Example 21, 70.0 mg, 140 μmol) in EtOH (3 mL) was added Pd/C (10%, 14.9 mg, 14.0 μmol) and the mixture was stirred under hydrogen atmosphere at rt overnight. The mixture was diluted with dichloromethane, filtered, and concentrated under reduced pressure. Purification by prep. TLC (dichloromethane/EtOH) gave the titled compound (54.3 mg, 95% purity, 73% yield).

LC-MS (LC-MS METHOD 2): Rₜ=1.29 min; MS (ESIpos): m/z=502 [M+H]⁺

1H NMR (DMSO-d6) δ: 8.49 (s, 2H), 7.74 (d, 1H), 7.54 (d, 1H), 7.36 (m, 1H), 5.70 (br m, 1H), 4.61 (br d, 1H), 3.97 (s, 4H), 3.27-3.32 (m, 1H), 3.06-3.22 (m, 2H), 2.67 (br d, 1H), 2.61 (s, 4H), 2.32 (s, 4H), 2.05 (s, 3H), 1.80-1.93 (m, 2H), 1.51-1.67 (m, 5H)

### Example 23

### 2-[7-methoxy-2-methyl-4-({(1R)-1-[2-methyl-3-(trifluoromethyl)phenyl]ethyl}amino)pyrido[2,3-d]pyrimidin-6-yl]-1 lambda⁶,2-thiazolidine-1,1-dione

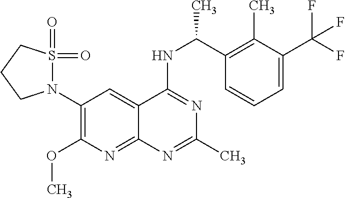 [SMILES: COc1nc2nc(C)nc(N[C@H](C)c3cccc(C(F)(F)F)c3C)c2cc1N1CCCS1(=O)=O]

To a solution of 6-bromo-7-methoxy-2-methyl-N-{(1R)-1-[2-methyl-3-(trifluoromethyl)phenyl]-ethyl}pyrido[2,3-d]pyrimidin-4-amine (Intermediate 38, 125 mg, 275 μmol) and 1,1-dioxoisothiazolidine (49.9 mg, 412 μmol) in 1,4-dioxane (3.1 mL) was added potassium phosphate (117 mg, 549 μmol), N,N-dimethylethylenediamine (48 μL, 440 μmol) and copper(I) iodide (41.8 mg, 220 μmol) and the mixture was stirred at 90° C. overnight. Purification by HPLC (basic method) gave the titled compound (12.5 mg, 98% purity, 9% yield).

LC-MS (LC-MS METHOD 2): Rₜ=1.25 min; MS (ESIpos): m/z=496 [M+H]⁺

¹H-NMR (400 MHz, DMSO-d6) δ[ppm]: 1.527 (3.91), 1.544 (3.96), 2.318 (0.44), 2.342 (14.32), 2.451 (0.85), 2.455 (0.83), 2.468 (1.58), 2.473 (1.68), 2.518 (4.39), 2.523 (3.33), 2.615 (4.84), 2.660 (0.44), 3.373 (1.13), 3.377 (1.03), 3.392 (2.27), 3.396 (2.20), 3.410 (1.00), 3.414 (1.06), 3.713 (0.51), 3.721 (0.72), 3.737 (1.15), 3.753 (0.94), 3.769 (1.33), 3.775 (0.44), 3.786 (0.70), 3.793 (0.62), 3.970 (16.00), 5.678 (0.59), 5.696 (0.94), 5.713 (0.59), 7.343 (0.57), 7.362 (1.21), 7.382 (0.71), 7.533 (1.33), 7.551 (1.05), 7.752 (1.17), 7.772 (1.04), 8.683 (1.06), 8.701 (1.17), 8.706 (5.02).

### Example 24

### 1-{6-[4-({(1R)-1-[3-(1,1-difluoro-2-hydroxyethyl)-2-fluorophenyl]ethyl}amino)-2-methyl-7-(trifluoromethyl)pyrido[2,3-d]pyrimidin-6-yl]-2,6-diazaspiro[3.3]heptan-2-yl}ethan-1-one

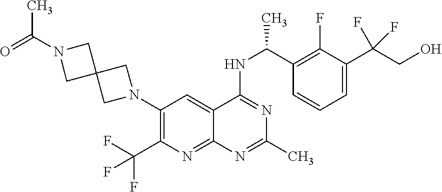 [SMILES: CC(=O)N1CC2(C1)CN(c1cc3c(N[C@H](C)c4cccc(C(F)(F)CO)c4F)nc(C)nc3nc1C(F)(F)F)C2]

To a solution of tert-butyl 6-[4-({(1R)-1-[3-(2-{[tert-butyl(dimethyl)silyl]oxy}-1,1-difluoroethyl)-2-fluorophenyl]ethyl}amino)-2-methyl-7-(trifluoromethyl)pyrido[2,3-d]pyrimidin-6-yl]-2,6-diazaspiro[3.3]heptane-2-carboxylate (Intermediate 11, 130 mg, 175 μmol) and triethyl silane (2.8 μL, 18 μmol) in dichloromethane (1.1 mL) was added trifluoroacetic acid (270 μL, 3.5 mmol) and the mixture was stirred at rt overnight. Then, toluene (1 mL) was added, and the solution was concentrated under reduced pressure. The residue was dissolved in dichloromethane (2 mL), N,N-diisopropylethylamine (67 μL, 390 μmol) and acetic anhydride (18 μL, 190 μmol) were added and the mixture was stirred at rt for 2 h. Toluene was added, and the mixture was concentrated under reduced pressure. The titled compound (24.0 mg, 95% purity, 23% yield) was obtained after purification by HPLC (basic method).

LC-MS (LC-MS METHOD 2): Rₜ=1.02 min; MS (ESIpos): m/z=569 [M+H]⁺

¹H-NMR (400 MHz, DMSO-d6) δ[ppm]: 1.286 (0.44), 1.304 (0.43), 1.615 (4.82), 1.633 (4.80), 1.762 (15.29), 2.322 (0.64), 2.326 (0.83), 2.332 (0.68), 2.347 (16.00), 2.518 (2.40), 2.522 (1.62), 2.664 (0.53), 2.668 (0.73), 2.673 (0.51), 3.894 (0.64), 3.910 (0.70), 3.930 (1.18), 3.946 (1.25), 3.966 (0.57), 3.982 (0.56), 4.059 (4.76), 4.218 (0.58), 4.244 (5.80), 4.268 (0.56), 4.343 (4.89), 5.710 (1.24), 5.718 (0.41), 5.726 (2.97), 5.742 (1.19), 5.775 (0.77), 5.794 (1.20), 5.812 (0.75), 7.245 (0.91), 7.264 (2.05), 7.283 (1.29), 7.414 (0.80), 7.431 (1.19), 7.448 (0.56), 7.611 (0.62), 7.629 (1.10), 7.646 (0.57), 7.991 (3.48), 8.798 (1.28), 8.816 (1.21).

### Example 25

### 1-{4-[2-methyl-4-({(1R)-1-[2-methyl-3-(trifluoromethyl)phenyl]ethyl}amino)pyrido[2,3-d]pyrimidin-6-yl]-3,6-dihydropyridin-1(2H)-yl}ethan-1-one

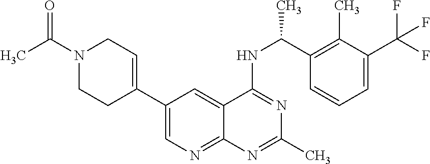 [SMILES: CC(=O)N1CC=C(c2cnc3nc(C)nc(N[C@H](C)c4cccc(C(F)(F)F)c4C)c3c2)CC1]

Using the method described for Example 24: tert-butyl 4-[2-methyl-4-({(1R)-1-[2-methyl-3-(trifluoromethyl)phenyl]ethyl}amino)pyrido[2,3-d]pyrimidin-6-yl]-3,6-dihydropyridine-1(2H)-carboxylate (Intermediate 12, 80.7 mg, 153 μmol), triethyl silane (2.4 μL, 15 μmol), trifluoroacetic acid (180 μL, 2.3 mmol), N,N-diisopropylethylamine (59 μL, 340 μmol) and acetic anhydride (16 μL, 170 μmol) in dichloromethane (1 mL) gave the titled compound (58.1 mg, 95% purity, 77% yield) after purification by preparative TLC (dichloromethane/ethanol).

LC-MS (LC-MS METHOD 2): Rₜ=1.19 min; MS (ESIneg): m/z=468 [M−H]⁻

1H NMR (DMSO-d6) δ: 9.09 (m, 1H), 8.79-8.87 (m, 2H), 7.77 (d, 1H), 7.55 (d, 1H), 7.36 (m, 1H), 6.45-6.48 (m, 1H), 5.70-5.77 (m, 1H), 4.20 (br m, 2H), 3.69-3.77 (m, 2H), 2.59-2.75 (m, 5H), 2.52-2.58 (m, 1H), 2.32-2.38 (m, 3H), 2.09 (d, 3H), 1.58 (d, 3H)

### Example 26

### 1-{4-[2-methyl-4-({(1R)-1-[2-methyl-3-(trifluoromethyl)phenyl]ethyl}amino)pyrido[2,3-d]pyrimidin-6-yl]-3,6-dihydropyridin-1(2H)-yl}ethan-1-one hydrogen

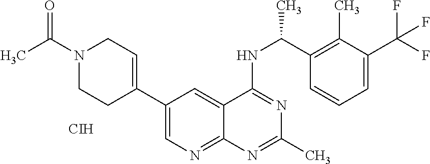

To a solution of 1-{4-[2-methyl-4-({(1R)-1-[2-methyl-3-(trifluoromethyl)phenyl]ethyl}amino)-pyrido[2,3-d]pyrimidin-6-yl]-3,6-dihydropyridin-1(2H)-yl}ethan-1-one (Example 25, 15.0 mg, 31.9 μmol) in 1,4-dioxane (500 μL) was added HCl in 1,4-dioxane (8.8 μL, 4.0 M, 35 μmol) and the mixture was stirred at rt for 10 min. The mixture was concentrated under reduced pressure and dried at 60° C. under reduced pressure to give the titled compound (15.9 mg, 95% purity, 93% yield).

¹H NMR (DMSO-d6) δ: 10.71 (br s, 1H), 9.27 (br s, 1H), 9.20 (m, 1H), 7.92 (d, 1H), 7.61 (d, 1H), 7.43 (m, 1H), 6.58-6.63 (m, 1H), 5.92 (m, 1H), 4.22 (br m, 2H), 3.72 (m, 2H), 3.56 (s, 1H), 2.63-2.75 (m, 2H), 2.52-2.62 (m, 7H), 2.09 (d, 3H), 1.68 (d, 3H)

### Example 27

### 1-{7-[2-methyl-4-({(1R)-1-[2-methyl-3-(trifluoromethyl)phenyl]ethyl}amino)pyrido[2,3-d]pyrimidin-6-yl]-2-azaspiro[3.5]non-6-en-2-yl}ethan-1-one

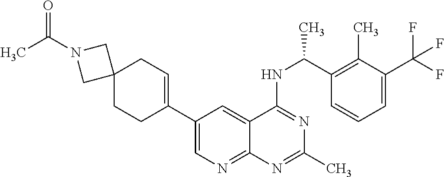 [SMILES: CC(=O)N1CC2(CC=C(c3cnc4nc(C)nc(N[C@H](C)c5cccc(C(F)(F)F)c5C)c4c3)CC2)C1]

Using the method described for Example 24: tert-butyl 7-[2-methyl-4-({(1R)-1-[2-methyl-3-(trifluoromethyl)phenyl]ethyl}amino)pyrido[2,3-d]pyrimidin-6-yl]-2-azaspiro[3.5]non-6-ene-2-carboxylate (Intermediate 13, 128 mg, 225 μmol), trifluoroacetic acid (260 μL, 3.4 mmol), N,N-diisopropylethylamine (86 μL, 500 μmol) and acetic anhydride (23 μL, 250 μmol) in dichloromethane (1.5 mL) gave the titled compound (19.7 mg, 97% purity, 17% yield) after purification by HPLC (basic method).

LC-MS (LC-MS METHOD 2): Rₜ=1.25 min; MS (ESIpos): m/z=510 [M+H]⁺

¹H NMR (DMSO-d6) δ: 9.07 (m, 1H), 8.83 (d, 1H), 8.77 (d, 1H), 7.77 (d, 1H), 7.55 (d, 1H), 7.36 (m, 1H), 6.38-6.42 (m, 1H), 5.73 (m, 1H), 3.93 (d, 1H), 3.84 (d, 1H), 3.66 (d, 1H), 3.57 (d, 1H), 2.58-2.68 (m, 5H), 2.52-2.54 (m, 1H), 2.32-2.39 (m, 4H), 1.91-2.01 (m, 2H), 1.77 (s, 3H), 1.57 (d, 3H), 1.23 (s, 1H)

### Example 28

### 1-{7-[2-methyl-4-({(1R)-1-[2-methyl-3-(trifluoromethyl)phenyl]ethyl}amino)pyrido[2,3-d]pyrimidin-6-yl]-2-azaspiro[3.5]nonan-2-yl}ethan-1-one

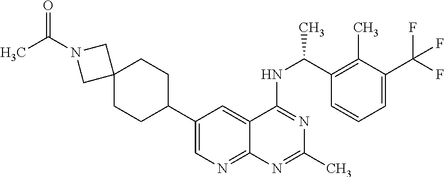 [SMILES: CC(=O)N1CC2(CCC(c3cnc4nc(C)nc(N[C@H](C)c5cccc(C(F)(F)F)c5C)c4c3)CC2)C1]

Using the method described for Example 22: 1-{7-[2-methyl-4-({(1R)-1-[2-methyl-3-(trifluoromethyl)phenyl]ethyl}amino)pyrido[2,3-d]pyrimidin-6-yl]-2-azaspiro[3.5]non-6-en-2-yl}ethan-1-one (Example 27, 140 mg, 275 μmol) and Pd/C (10%, 29.2 mg, 27.5 μmol) in ethanol (6.0 mL) for 3 h gave the titled compound (35.0 mg, 90% purity, 22% yield) after purification by HPLC (basic method).

LC-MS (LC-MS METHOD 2): Rₜ=1.24 min; MS (ESIpos): m/z=512 [M+H]⁺

¹H-NMR (400 MHz, DMSO-d6) δ[ppm]: 1.533 (0.70), 1.561 (6.63), 1.579 (6.32), 1.602 (1.53), 1.635 (1.45), 1.668 (0.56), 1.765 (8.18), 1.771 (1.48), 1.785 (10.05), 1.862 (1.18), 1.894 (1.16), 1.988 (1.29), 2.016 (1.01), 2.318 (0.45), 2.322 (1.01), 2.326 (1.46), 2.331 (1.04), 2.336 (0.57), 2.354 (16.00), 2.364 (1.17), 2.518 (5.11), 2.522 (3.46), 2.621 (7.43), 2.660 (0.79), 2.664 (1.41), 2.668 (1.66), 2.673 (1.27), 2.678 (0.78), 2.684 (0.63), 2.692 (0.68), 3.515 (3.68), 3.637 (3.32), 3.786 (3.75), 3.910 (3.82), 5.702 (0.88), 5.720 (1.38), 5.737 (0.87), 7.341 (0.80), 7.361 (1.76), 7.381 (1.02), 7.535 (1.97), 7.554 (1.57), 7.760 (1.77), 7.780 (1.59), 8.592 (2.05), 8.597 (2.06), 8.709 (0.80), 8.717 (0.94), 8.726 (0.88), 8.735 (0.84), 8.853 (2.08), 8.856 (2.49), 8.858 (2.46), 8.862 (1.90).

### Example 29

### 1-{6-[4-({(1R)-1-[3-(difluoromethyl)-2-fluorophenyl]ethyl}amino)-2-methylpyrido[2,3-d]pyrimidin-6-yl]-2,6-diazaspiro[3.3]heptan-2-yl}ethan-1-one

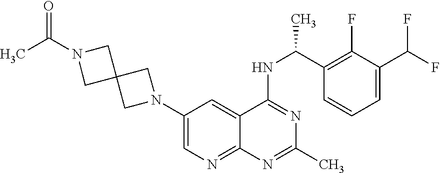 [SMILES: CC(=O)N1CC2(C1)CN(c1cnc3nc(C)nc(N[C@H](C)c4cccc(C(F)F)c4F)c3c1)C2]

Using the method described for Example 7: 6-bromo-N-{(1R)-1-[3-(difluoromethyl)-2-fluorophenyl]ethyl}-2-methylpyrido[2,3-d]pyrimidin-4-amine (Intermediate 42, 100 mg, 243 μmol), oxalic acid 1-(2,6-diazaspiro[3.3]heptan-2-yl)ethan-1-one (½) (180 mg, 486 μmol), sodium tert-butoxide (117 mg, 1.22 mmol), Pd₂dba₃ (19.1 mg, 24.3 μmol), XPhos (23.2 mg, 48.6 μmol) in 1,4-dioxane (3.0 mL) at 100° C. overnight gave the titled compound (12.4 mg, 95% purity, 10% yield) after purification by HPLC and prep. TLC (dichloromethane/EtOH).

LC-MS (LC-MS METHOD 2): Rₜ=1.02 min; MS (ESIneg): m/z=469 [M−H]⁻

¹H-NMR (400 MHz, DMSO-d6) δ[ppm]: 1.231 (0.50), 1.595 (5.70), 1.612 (5.67), 1.766 (15.42), 2.314 (16.00), 2.327 (1.73), 2.331 (1.18), 2.523 (4.87), 2.665 (1.02), 2.669 (1.35), 2.673 (0.98), 4.060 (5.65), 4.126 (0.86), 4.149 (9.66), 4.174 (0.80), 4.338 (5.82), 5.754 (0.90), 5.772 (1.38), 5.790 (0.90), 7.101 (1.24), 7.236 (2.60), 7.267 (1.00), 7.286 (2.17), 7.305 (1.25), 7.372 (1.11), 7.481 (0.81), 7.498 (1.34), 7.516 (0.66), 7.628 (0.76), 7.646 (1.36), 7.664 (0.71), 7.687 (2.60), 7.694 (2.61), 8.356 (3.44), 8.363 (3.29), 8.387 (1.55), 8.405 (1.47).

### Example 30

### 1-{3-[2-methyl-4-({(1R)-1-[2-methyl-3-(trifluoromethyl)phenyl]ethyl}amino)pyrido[2,3-d]pyrimidin-6-yl]-2,5-dihydro-1H-pyrrol-1-yl}ethan-1-one

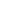

Using the method described for Example 24: tert-butyl 3-[2-methyl-4-({(1R)-1-[2-methyl-3-(trifluoromethyl)phenyl]ethyl}amino)pyrido[2,3-d]pyrimidin-6-yl]-2,5-dihydro-1H-pyrrole-1-carboxylate (265 mg, 516 μmol), triethyl silane (8.2 μL, 52 μmol), trifluoroacetic acid (600 μL, 7.7 mmol), N,N-diisopropylethylamine (200 μL, 1.1 mmol) and acetic anhydride (54 μL, 570 μmol) in dichloromethane (4 mL) gave the titled compound (167 mg, 95% purity, 68% yield) after purification by HPLC (basic method).

LC-MS (LC-MS METHOD 2): Rₜ=1.17 min; MS (ESIneg): m/z=454 [M−H]⁻

1H NMR (DMSO-d6) δ: 9.32 (d, 2H), 9.19 (d, 1H), 8.94 (d, 2H), 8.82 (d, 1H), 8.76 (d, 1H), 8.68 (d, 2H), 7.72-7.79 (m, 3H), 7.55 (d, 3H), 7.37 (m, 3H), 6.70-6.76 (m, 3H), 5.68-5.77 (m, 3H), 4.75-4.81 (m, 2H), 4.59 (br s, 4H), 4.50-4.56 (m, 4H), 4.31 (br s, 2H), 2.62 (s, 9H), 2.52-2.59 (m, 1H), 2.33-2.42 (m, 9H), 2.12 (s, 3H), 2.05 (s, 6H), 1.55-1.62 (m, 9H)

### Example 31

### 1-{(3RS)-3-[2-methyl-4-({(1R)-1-[2-methyl-3-(trifluoromethyl)phenyl]ethyl}amino)pyrido[2,3-d]pyrimidin-6-yl]pyrrolidin-1-yl}ethan-1-one

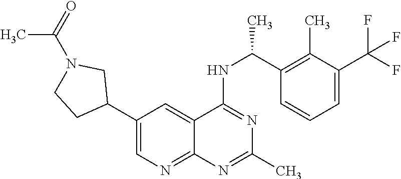 [SMILES: CC(=O)N1CCC(c2cnc3nc(C)nc(N[C@H](C)c4cccc(C(F)(F)F)c4C)c3c2)C1]

Using the method described for Example 22: 1-{3-[2-methyl-4-({(1R)-1-[2-methyl-3-(trifluoromethyl)phenyl]ethyl}amino)pyrido[2,3-d]pyrimidin-6-yl]-2,5-dihydro-1H-pyrrol-1-yl}ethan-1-one (134 mg, 294 μmol) and Pd/C (10%, 31.3 mg, 29.4 μmol) in ethanol (5.0 mL) for 6 h gave the titled compound (62.0 mg, 95% purity, 44% yield) after purification by HPLC (basic method) and prep. TLC (dichloromethane/EtOH).

LC-MS (LC-MS METHOD 2): Rₜ=1.15 min; MS (ESIpos): m/z=458 [M+H]⁺

1H NMR (DMSO-d6) δ: 8.87-8.92 (m, 2H), 8.73-8.82 (m, 4H), 7.76 (br m, 2H), 7.55 (br d, 2H), 7.36 (m, 2H), 5.69-5.77 (m, 3H), 3.97-4.05 (m, 2H), 3.66-3.78 (m, 2H), 3.44-3.64 (m, 4H), 3.35-3.39 (m, 1H), 2.60-2.71 (m, 7H), 2.53-2.57 (m, 4H), 2.30-2.44 (m, 9H), 1.98-2.16 (m, 9H), 1.90-1.97 (m, 1H), 1.57 (d, 6H)

### Example 32

### 6-methoxy-2-methyl-N-{(1R)-1-[2-methyl-3-(trifluoromethyl)phenyl]ethyl}pyrido[2,3-d]pyrimidin-4-amine

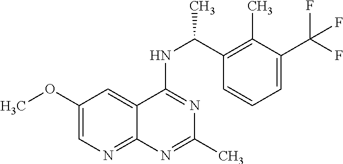 [SMILES: COc1cnc2nc(C)nc(N[C@H](C)c3cccc(C(F)(F)F)c3C)c2c1]

To a solution of 6-bromo-2-methyl-N-{(1R)-1-[2-methyl-3-(trifluoromethyl)-phenyl]ethyl}pyrido[2,3-d]pyrimidin-4-amine (75.0 mg, 176 μmol) and copper(I) iodide (16.8 mg, 88.2 μmol) in DMF (1.0 mL) was added a solution of sodium methoxide in methanol (130 μL, 5.4 M, 710 μmol) and the mixture was stirred at 105° C. for 2 h. Purification by HPLC (basic method) gave the titled compound (21.0 mg, 95% purity, 30% yield).

LC-MS (LC-MS METHOD 2): Rₜ=1.21 min; MS (ESIpos): m/z=377 [M+H]⁺

¹H-NMR (400 MHz, DMSO-d6) δ[ppm]: 1.567 (4.41), 1.584 (4.39), 2.074 (0.55), 2.331 (1.33), 2.337 (0.73), 2.350 (15.55), 2.518 (6.72), 2.523 (4.67), 2.625 (4.93), 2.673 (1.21), 2.678 (0.54), 3.960 (16.00), 5.703 (0.63), 5.720 (0.99), 5.737 (0.63), 7.343 (0.56), 7.362 (1.23), 7.381 (0.71), 7.537 (1.30), 7.555 (1.06), 7.755 (1.18), 7.774 (1.05), 8.275 (2.10), 8.283 (2.14), 8.618 (0.99), 8.636 (0.98), 8.683 (3.34), 8.691 (3.26).

### Example 33

### N-methyl-N-[2-methyl-4-({(1R)-1-[2-methyl-3-(trifluoromethyl)phenyl]ethyl}amino)pyrido[2,3-d]pyrimidin-6-yl]methanesulfonamide

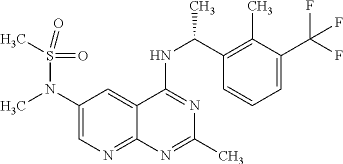 [SMILES: Cc1nc(N[C@H](C)c2cccc(C(F)(F)F)c2C)c2cc(N(C)S(C)(=O)=O)cnc2n1]

To a solution of 6-bromo-2-methyl-N-{(1R)-1-[2-methyl-3-(trifluoromethyl)phenyl]ethyl}-pyrido[2,3-d]pyrimidin-4-amine (100 mg, 235 μmol) and N-methylmethanesulfonamide (38.5 mg, 353 μmol) in 1,4-dioxane (1 mL) were added potassium carbonate (65.0 mg, 470 μmol), trans-N,N-dimethylcyclohexane-1,2-diamine (7.4 μL, 47 μmol) and copper(I) iodide (8.96 mg, 47.0 μmol) and the mixture was stirred at 110° C. overnight. The mixture was diluted with dichloromethane, filtered, and concentrated under reduced pressure. Purification by flash column chromatography and prep. TLC (dichloromethane/EtOH) gave the titled compound (18.0 mg, 95% purity, 16% yield).

LC-MS (LC-MS METHOD 2): Rₜ=1.19 min; MS (ESIpos): m/z=454 [M+H]⁺

¹H-NMR (400 MHz, DMSO-d6) δ[ppm]: 1.154 (0.40), 1.172 (0.82), 1.564 (3.78), 1.582 (3.78), 1.988 (1.29), 2.331 (1.24), 2.336 (0.56), 2.382 (9.74), 2.518 (6.85), 2.523 (4.84), 2.620 (4.57), 2.673 (1.22), 2.678 (0.55), 3.119 (13.93), 3.378 (16.00), 5.700 (0.56), 5.717 (0.86), 5.735 (0.56), 5.759 (3.19), 7.345 (0.52), 7.364 (1.13), 7.384 (0.64), 7.544 (1.22), 7.562 (0.99), 7.750 (1.08), 7.769 (0.99), 8.853 (1.64), 8.860 (2.02), 8.881 (0.82), 8.960 (1.03), 8.965 (0.96).

### Example 34

### 2-[2-methyl-4-({(1R)-1-[2-methyl-3-(trifluoromethyl)phenyl]ethyl}amino)pyrido[2,3-d]pyrimidin-6-yl]-1 lambda⁶,2-thiazolidine-1,1-dione

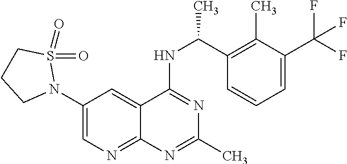 [SMILES: Cc1nc(N[C@H](C)c2cccc(C(F)(F)F)c2C)c2cc(N3CCCS3(=O)=O)cnc2n1]

To a solution of 6-bromo-2-methyl-N-{(1R)-1-[2-methyl-3-(trifluoromethyl)phenyl]ethyl}-pyrido[2,3-d]pyrimidin-4-amine (100 mg, 235 μmol) and 1,1-dioxoisothiazolidine (42.7 mg, 353 μmol) in 1,4-dioxane (2.0 mL) was added caesium carbonate (115 mg, 353 μmol), Xantphos (20.4 mg, 35.3 μmol) and palladium(II) acetate (5.28 mg, 23.5 μmol) and the mixture was stirred at 100° C. overnight. The mixture was diluted with dichloromethane, filtered, and concentrated under reduced pressure. Purification by HPLC (basic method) and preparative TLC (dichloromethane/MeOH) gave the titled compound (15.0 mg, 95% purity, 13% yield).

LC-MS (LC-MS METHOD 2): Rₜ=1.19 min; MS (ESIpos): m/z=466 [M+H]⁺

¹H-NMR (400 MHz, DMSO-d6) δ[ppm]: 1.052 (0.79), 1.232 (0.62), 1.569 (4.58), 1.586 (4.49), 2.331 (1.12), 2.336 (0.50), 2.371 (16.00), 2.518 (7.94), 2.523 (4.72), 2.619 (5.47), 2.673 (1.08), 2.678 (0.48), 3.604 (2.03), 3.623 (3.83), 3.641 (1.87), 3.875 (0.55), 3.882 (0.89), 3.898 (2.27), 3.915 (2.24), 3.931 (0.82), 3.937 (0.56), 5.710 (0.68), 5.727 (1.06), 5.744 (0.68), 5.760 (1.60), 7.344 (0.60), 7.363 (1.36), 7.383 (0.79), 7.543 (1.48), 7.561 (1.18), 7.750 (1.31), 7.769 (1.20), 8.471 (2.33), 8.479 (2.34), 8.837 (1.13), 8.854 (1.12), 8.932 (3.68), 8.939 (3.65).

### Example 35

### 1-{4-[2-methyl-4-({(1R)-1-[2-methyl-3-(trifluoromethyl)phenyl]ethyl}amino)pyrido[2,3-d]pyrimidin-6-yl]piperazin-1-yl}ethan-1-one

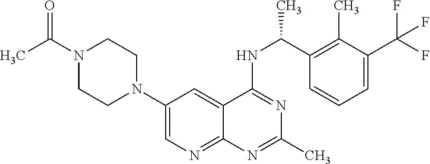 [SMILES: CC(=O)N1CCN(c2cnc3nc(C)nc(N[C@H](C)c4cccc(C(F)(F)F)c4C)c3c2)CC1]

Using the method described for Example 7: 6-bromo-2-methyl-N-{(1R)-1-[2-methyl-3-(trifluoromethyl)phenyl]ethyl}pyrido[2,3-d]pyrimidin-4-amine (40.0 mg, 94.1 μmol), 1-(piperazin-1-yl)ethan-1-one (24.1 mg, 188 μmol), sodium tert-butoxide (18.1 mg, 188 μmol), Pd₂dba₃ (4.31 mg, 4.70 μmol), XPhos (4.48 mg, 9.41 μmol) in 1,4-dioxane (890 μL) at 100° C. for 24 h gave the titled compound (17.0 mg, 95% purity, 36% yield) after purification by HPLC (basic method).

LC-MS (LC-MS METHOD 2): Rₜ=1.17 min; MS (ESIpos): m/z=474 [M+H]⁺

¹H-NMR (400 MHz, DMSO-d6) δ[ppm]: 1.557 (4.73), 1.575 (4.70), 2.075 (16.00), 2.328 (15.81), 2.518 (2.49), 2.523 (1.58), 2.540 (5.74), 2.620 (5.98), 2.665 (0.48), 2.669 (0.65), 2.673 (0.46), 3.254 (0.73), 3.266 (1.34), 3.278 (1.39), 3.287 (0.96), 3.363 (1.77), 3.637 (1.42), 3.651 (2.99), 3.663 (3.23), 3.677 (1.21), 5.699 (0.72), 5.716 (1.11), 5.734 (0.71), 7.336 (0.68), 7.355 (1.47), 7.375 (0.85), 7.532 (1.60), 7.550 (1.30), 7.743 (1.43), 7.762 (1.28), 8.065 (1.95), 8.073 (1.93), 8.543 (1.23), 8.561 (1.25), 8.870 (2.59), 8.878 (2.54).

### Example 36

### 1-{4-[2-methyl-4-({(1R)-1-[2-methyl-3-(trifluoromethyl)phenyl]ethyl}amino)pyrido[2,3-d]pyrimidin-6-yl]-4-oxo-4lambda⁵-piperazin-1-yl}ethan-1-one

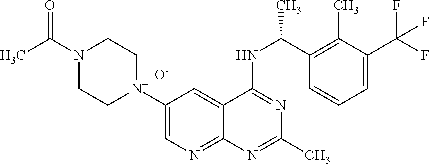

To a solution of 1-{4-[2-methyl-4-({(1R)-1-[2-methyl-3-(trifluoromethyl)phenyl]ethyl}amino)-pyrido[2,3-d]pyrimidin-6-yl]piperazin-1-yl}ethan-1-one (115 mg, 243 μmol) in dichloromethane (5.0 mL) was added 3-chlorobenzene-1-carboperoxoic acid (75%, 168 mg, 730 μmol) and the mixture was stirred at rt overnight. The mixture was diluted with dichloromethane and washed with saturated aqueous sodium hydrogen carbonate solution. The aqueous phase was basified with Et₃N and extracted with dichloromethane/MeOH 9:1. The combined org. phases were washed with brine, dried over Na₂SO₄, filtered, and concentrated under reduced pressure. Purification by HPLC (basic method) gave the titled compound (32.3 mg, 95% purity, 26% yield).

LC-MS (LC-MS METHOD 2): Rₜ=1.00 min; MS (ESIpos): m/z=489 [M+H]⁺

¹H-NMR (400 MHz, DMSO-d6) δ[ppm]: 1.564 (5.44), 1.582 (5.37), 2.109 (16.00), 2.323 (0.96), 2.327 (1.43), 2.332 (1.02), 2.401 (13.76), 2.518 (7.82), 2.523 (4.97), 2.624 (7.60), 2.665 (1.00), 2.669 (1.34), 2.673 (0.99), 3.057 (1.40), 3.079 (1.41), 3.382 (0.51), 3.595 (0.46), 3.624 (0.88), 3.654 (0.59), 3.880 (0.89), 3.909 (0.93), 3.980 (0.48), 4.009 (0.80), 4.038 (0.41), 4.085 (0.80), 4.117 (1.86), 4.149 (0.88), 4.437 (0.85), 4.469 (0.78), 5.711 (0.82), 5.729 (1.24), 5.746 (0.81), 7.343 (0.82), 7.363 (1.80), 7.383 (1.05), 7.542 (1.98), 7.561 (1.62), 7.787 (1.75), 7.806 (1.59), 9.141 (0.79), 9.150 (0.96), 9.157 (0.92), 9.167 (0.78), 9.631 (2.02), 9.637 (4.06), 9.649 (2.99), 9.655 (1.64).

### Example 37

### 1-{6-[4-({(1R)-1-[3-(difluoromethyl)-2-fluorophenyl]ethyl}amino)-2,7-dimethylpyrido[2,3-d]pyrimidin-6-yl]-2,6-diazaspiro[3.3]heptan-2-yl}ethan-1-one

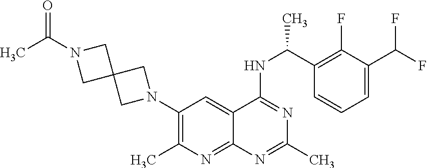 [SMILES: CC(=O)N1CC2(C1)CN(c1cc3c(N[C@H](C)c4cccc(C(F)F)c4F)nc(C)nc3nc1C)C2]

Using the method described for Example 7: 6-bromo-N-{(1R)-1-[3-(difluoromethyl)-2-fluorophenyl]ethyl}-2,7-dimethylpyrido[2,3-d]pyrimidin-4-amine (Intermediate 6, 100 mg, 235 μmol), oxalic acid 1-(2,6-diazaspiro[3.3]heptan-2-yl)ethan-1-one (½) (174 mg, 470 μmol), sodium tert-butoxide (90.4 mg, 941 μmol), Pd₂dba₃ (18.5 mg, 23.5 μmol), XPhos (22.4 mg, 47.0 μmol) in 1,4-dioxane (2.2 mL) gave the titled compound (15.3 mg, 95% purity, 13% yield) after purification by prep. TLC (dichloromethane/MeOH 9:1).

LC-MS (LC-MS METHOD 2): Rₜ=1.02 min; MS (ESIpos): m/z=485 [M+H]⁺

¹H-NMR (400 MHz, DMSO-d6) δ[ppm]: 1.595 (4.82), 1.612 (4.83), 1.719 (0.73), 1.772 (14.88), 2.303 (16.00), 2.318 (0.53), 2.463 (15.06), 2.518 (4.30), 2.523 (3.00), 4.052 (4.70), 4.113 (0.88), 4.135 (5.83), 4.141 (5.56), 4.162 (0.81), 4.332 (4.89), 5.763 (0.75), 5.781 (1.14), 5.799 (0.73), 7.102 (1.12), 7.238 (2.40), 7.268 (0.85), 7.287 (1.82), 7.306 (1.05), 7.374 (0.98), 7.481 (0.63), 7.497 (1.05), 7.516 (0.51), 7.634 (4.31), 7.652 (1.07), 7.669 (0.53), 8.375 (1.24), 8.393 (1.20).

### Example 38

### 6-methoxy-2,7-dimethyl-N-{(1R)-1-[2-methyl-3-(trifluoromethyl)phenyl]ethyl}pyrido[2,3-d]pyrimidin-4-amine

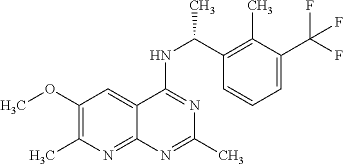 [SMILES: COc1cc2c(N[C@H](C)c3cccc(C(F)(F)F)c3C)nc(C)nc2nc1C]

Using the method described for Example 32: 6-bromo-2,7-dimethyl-N-{(1R)-1-[2-methyl-3-(trifluoromethyl)phenyl]ethyl}pyrido[2,3-d]pyrimidin-4-amine (75.0 mg, 171 μmol), copper(I) iodide (16.3 mg, 85.4 μmol) and a solution of sodium methoxide in methanol (130 μL, 5.4 M, 680 μmol) in DMF (1.0 mL) gave the titled compound (17.0 mg, 95% purity, 24% yield) after purification by HPLC (basic method).

LC-MS (LC-MS METHOD 2): Rₜ=1.27 min; MS (ESIneg): m/z=389 [M−H]⁻

¹H-NMR (400 MHz, DMSO-d6) δ[ppm]: 1.560 (3.85), 1.577 (3.83), 2.326 (16.00), 2.481 (14.47), 2.518 (5.85), 2.522 (3.85), 2.621 (4.31), 2.660 (0.41), 2.664 (0.94), 2.669 (1.26), 2.673 (0.92), 2.678 (0.40), 3.970 (12.12), 5.694 (0.56), 5.712 (0.86), 5.730 (0.56), 7.338 (0.50), 7.358 (1.09), 7.378 (0.63), 7.532 (1.17), 7.549 (0.94), 7.749 (1.03), 7.768 (0.94), 8.135 (3.18), 8.508 (0.88), 8.526 (0.85).

### Example 39

### 2-[4-({(1R)-1-[3-(difluoromethyl)-2-fluorophenyl]ethyl}amino)-2,7-dimethylpyrido[2,3-d]pyrimidin-6-yl]-1 lambda⁶,2-thiazolidine-1,1-dione

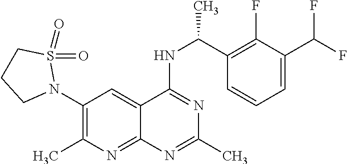 [SMILES: Cc1nc(N[C@H](C)c2cccc(C(F)F)c2F)c2cc(N3CCCS3(=O)=O)c(C)nc2n1]

Using the method described for Example 33: 6-bromo-N-{(1R)-1-[3-(difluoromethyl)-2-fluorophenyl]ethyl}-2,7-dimethylpyrido[2,3-d]pyrimidin-4-amine (100 mg, 235 μmol), 1,1-dioxoisothiazolidine (42.7 mg, 353 μmol), copper(I) iodide (8.96 mg, 47.0 μmol), trans —N,N-dimethylcyclohexane-1,2-diamine (7.3 μL, 47 μmol) and potassium carbonate (65.0 mg, 470 μmol) in 1,4-dioxane (1 mL) at 110° C. overnight gave the titled compound (7.00 mg, 95% purity, 6% yield) after purification by HPLC (basic method) and prep. TLC (dichloromethane/EtOH 9:1).

LC-MS (LC-MS METHOD 2): Rₜ=1.07 min; MS (ESIpos): m/z=466 [M+H]⁺

¹H-NMR (400 MHz, DMSO-d6) δ[ppm]: 1.232 (1.26), 1.606 (3.69), 1.624 (3.66), 2.332 (3.05), 2.336 (1.33), 2.374 (13.83), 2.518 (16.00), 2.522 (10.42), 2.629 (12.04), 2.673 (3.07), 2.678 (1.31), 3.499 (1.68), 3.518 (2.35), 3.537 (1.40), 3.750 (0.89), 3.767 (1.79), 3.786 (0.87), 5.766 (0.54), 5.784 (0.86), 5.801 (0.56), 7.107 (0.82), 7.242 (1.82), 7.287 (0.59), 7.307 (1.37), 7.326 (0.78), 7.378 (0.76), 7.497 (0.45), 7.514 (0.75), 7.651 (0.41), 7.668 (0.74), 7.687 (0.41), 8.775 (0.92), 8.794 (0.88), 8.857 (3.62).

### Example 40

### 1-{3-[2-methyl-4-({(1R)-1-[2-methyl-3-(trifluoromethyl)phenyl]ethyl}amino)pyrido[2,3-d]pyrimidin-6-yl]azetidin-1-yl}ethan-1-one

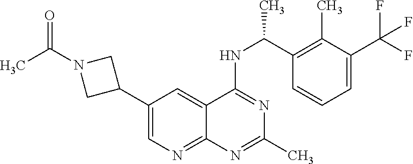 [SMILES: CC(=O)N1CC(c2cnc3nc(C)nc(N[C@H](C)c4cccc(C(F)(F)F)c4C)c3c2)C1]

Using the method described for Example 24: tert-butyl 3-[2-methyl-4-({(1R)-1-[2-methyl-3-(trifluoromethyl)phenyl]ethyl}amino)pyrido[2,3-d]pyrimidin-6-yl]azetidine-1-carboxylate (62.0 mg, 124 μmol), triethyl silane (2.0 μL, 12 μmol), trifluoroacetic acid (191 μL), N,N-diisopropylethylamine (110 μL, 620 μmol) and acetic anhydride (13 μL, 140 μmol) in dichloromethane (1.5 mL) gave the titled compound (26.6 mg, 95% purity, 46% yield) after purification by HPLC (basic method).

LC-MS (LC-MS METHOD 2): Rₜ=1.13 min; MS (ESIpos): m/z=444 [M+H]⁺

¹H NMR (DMSO-d6) δ: 8.90 (m, 1H), 8.80-8.86 (m, 2H), 7.76 (d, 1H), 7.55 (d, 1H), 7.36 (m, 1H), 5.72 (m, 1H), 4.56-4.61 (m, 1H), 4.21-4.34 (m, 2H), 4.03-4.14 (m, 2H), 2.62 (s, 3H), 2.52-2.54 (m, 1H), 2.34-2.38 (m, 3H), 1.82-1.85 (m, 3H), 1.58 (d, 3H)

### Example 41

### 1-{4-[2-methyl-4-({(1R)-1-[2-methyl-3-(trifluoromethyl)phenyl]ethyl}amino)-7-(trifluoromethyl)pyrido[2,3-d]pyrimidin-6-yl]-3,6-dihydropyridin-1(2H)-yl}ethan-1-one

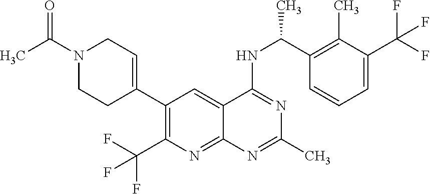 [SMILES: CC(=O)N1CC=C(c2cc3c(N[C@H](C)c4cccc(C(F)(F)F)c4C)nc(C)nc3nc2C(F)(F)F)CC1]

Using the method described for Example 5: 6-bromo-2-methyl-N-{(1R)-1-[2-methyl-3-(trifluoromethyl)phenyl]ethyl}-7-(trifluoromethyl)pyrido[2,3-d]pyrimidin-4-amine (70.0 mg, 142 μmol), 1-[4-(4,4,5,5-tetramethyl-1,3,2-dioxaborolan-2-yl)-3,6-dihydropyridin-1(2H)-yl]ethan-1-one (53.5 mg, 213 μmol), XPhosPdG2 (5.58 mg, 7.10 μmol) and aq. K₃PO₄ solution (570 μL, 0.50 M, 280 μmol) in 1,4-dioxane (3.5 mL) gave the titled compound (63.1 mg, 95% purity, 83% yield) after purification by flash column chromatography.

LC-MS (LC-MS METHOD 2): Rₜ=1.35 min; MS (ESIpos): m/z=538 [M+H]⁺

¹H-NMR (400 MHz, DMSO-d6) δ[ppm]: 1.052 (0.48), 1.065 (16.00), 1.556 (0.78), 1.574 (0.77), 2.070 (1.05), 2.081 (0.47), 2.103 (1.42), 2.409 (2.78), 2.518 (1.68), 2.523 (1.08), 2.614 (1.21), 3.939 (2.65).

### Example 42

### 1-{4-[2-methyl-4-({(1R)-1-[2-methyl-3-(trifluoromethyl)phenyl]ethyl}amino)-7-(trifluoromethyl)pyrido[2,3-d]pyrimidin-6-yl]piperidin-1-yl}ethan-1-one

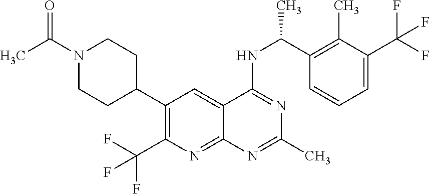 [SMILES: CC(=O)N1CCC(c2cc3c(N[C@H](C)c4cccc(C(F)(F)F)c4C)nc(C)nc3nc2C(F)(F)F)CC1]

Using the method described for Example 22: 1-{4-[2-methyl-4-({(1R)-1-[2-methyl-3-(trifluoromethyl)phenyl]ethyl}amino)-7-(trifluoromethyl)pyrido[2,3-d]pyrimidin-6-yl]-3,6-dihydropyridin-1(2H)-yl}ethan-1-one (50.0 mg, 93.0 μmol) and Pd/C (10%, 9.90 mg, 9.30 μmol) in EtOH (2.0 mL) for 16 h gave the titled compound (22.0 mg, 95% purity, 42% yield) after purification by prep. TLC (dichloromethane/MeOH 9:1).

LC-MS (LC-MS METHOD 2): Rₜ=1.35 min; MS (ESIpos): m/z=540 [M+H]⁺

¹H-NMR (400 MHz, DMSO-d6) δ[ppm]: 1.230 (0.70), 1.585 (4.27), 1.602 (4.29), 1.745 (0.49), 1.777 (0.85), 1.802 (1.72), 1.822 (1.66), 1.854 (0.45), 2.078 (16.00), 2.318 (0.68), 2.323 (1.53), 2.327 (2.14), 2.331 (1.50), 2.337 (0.67), 2.382 (8.28), 2.388 (8.11), 2.409 (0.69), 2.518 (8.30), 2.523 (5.74), 2.614 (7.02), 2.660 (1.08), 2.665 (1.92), 2.669 (2.49), 2.673 (1.77), 2.678 (0.91), 3.166 (0.76), 3.198 (0.77), 3.230 (0.43), 4.015 (0.67), 4.047 (0.61), 4.642 (0.71), 4.673 (0.67), 5.695 (0.46), 5.713 (0.78), 5.725 (0.77), 5.741 (0.46), 7.360 (0.72), 7.379 (1.55), 7.399 (0.93), 7.552 (1.28), 7.571 (1.03), 7.728 (1.75), 7.747 (1.63), 9.076 (4.29), 9.094 (0.85), 9.102 (0.89), 9.119 (0.70).

### Example 43

### 1-{4-[4-({(1R)-1-[3-(difluoromethyl)-2-fluorophenyl]ethyl}amino)-2-methylpyrido[2,3-d]pyrimidin-6-yl]-3,6-dihydropyridin-1(2H)-yl}ethan-1-one

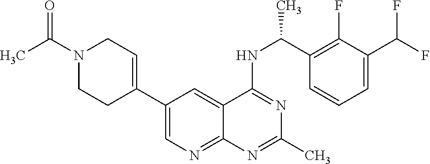 [SMILES: CC(=O)N1CC=C(c2cnc3nc(C)nc(N[C@H](C)c4cccc(C(F)F)c4F)c3c2)CC1]

Using the method described for Example 21: 6-bromo-N-{(1R)-1-[3-(difluoromethyl)-2-fluorophenyl]ethyl}-2-methylpyrido[2,3-d]pyrimidin-4-amine (Intermediate 42, 175 mg, 426 μmol), 1-[4-(4,4,5,5-tetramethyl-1,3,2-dioxaborolan-2-yl)-3,6-dihydropyridin-1(2H)-yl]ethan-1-one (128 mg, 511 μmol), bis(triphenylphosphine)palladium(II) chloride (29.9 mg, 43 μmol), potassium carbonate (88.2 mg, 638 μmol) in 1,2-dimethoxyethane (1.9 mL) and ethanol (1.9 mL) at 100° C. for 6 h gave the titled compound (153 mg, 90% purity, 71% yield)) after purification by flash column chromatography (dichloromethane/EtOH).

LC-MS (LC-MS METHOD 2): Rₜ=1.07 min; MS (ESIneg): m/z=454 [M−H]⁻

¹H-NMR (400 MHz, DMSO-d6) δ[ppm]: 0.797 (0.49), 0.814 (0.54), 0.821 (0.54), 0.904 (0.62), 1.066 (1.11), 1.156 (0.40), 1.620 (5.82), 1.638 (5.77), 2.068 (7.77), 2.107 (9.64), 2.323 (0.61), 2.327 (0.87), 2.332 (0.61), 2.393 (16.00), 2.518 (3.33), 2.523 (2.43), 2.599 (0.50), 2.614 (0.50), 2.660 (0.40), 2.665 (0.74), 2.669 (1.00), 2.673 (0.80), 2.679 (0.54), 2.705 (0.77), 2.729 (0.89), 2.888 (0.93), 3.691 (1.02), 3.706 (2.19), 3.719 (1.57), 3.732 (1.63), 3.746 (0.76), 4.176 (1.53), 4.184 (1.56), 4.224 (1.26), 4.231 (1.25), 5.759 (2.94), 5.795 (0.83), 5.813 (1.26), 5.831 (0.81), 6.474 (1.62), 7.107 (1.33), 7.242 (2.84), 7.283 (0.99), 7.302 (2.18), 7.321 (1.25), 7.378 (1.18), 7.500 (0.75), 7.517 (1.27), 7.534 (0.63), 7.669 (0.69), 7.687 (1.27), 7.706 (0.64), 8.820 (2.47), 9.097 (1.63), 9.103 (1.64), 9.129 (1.34), 9.135 (1.29).

### Example 44

### 1-{4-[4-({(1R)-1-[3-(difluoromethyl)-2-fluorophenyl]ethyl}amino)-2-methylpyrido[2,3-d]pyrimidin-6-yl]piperidin-1-yl}ethan-1-one

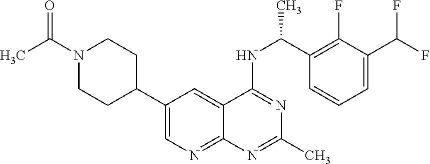 [SMILES: CC(=O)N1CCC(c2cnc3nc(C)nc(N[C@H](C)c4cccc(C(F)F)c4F)c3c2)CC1]

Using the method described for Example 22: 1-{4-[4-({(1R)-1-[3-(difluoromethyl)-2-fluorophenyl]ethyl}amino)-2-methylpyrido[2,3-d]pyrimidin-6-yl]-3,6-dihydropyridin-1(2H)-yl}ethan-1-one (Example 43, 52.0 mg, 114 μmol) and Pd/C (10%, 12.1 mg, 11.4 μmol) in EtOH (2.5 mL) for 4 h gave the titled compound (35.1 mg, 95% purity, 64% yield) after purification by HPLC (basic method).

LC-MS (LC-MS METHOD 2): Rₜ=1.06 min; MS (ESIpos): m/z=458 [M+H]⁺

¹H-NMR (400 MHz, DMSO-d6) δ[ppm]: 1.602 (4.60), 1.619 (4.91), 1.640 (0.88), 1.650 (0.69), 1.672 (0.68), 1.682 (0.44), 1.866 (0.60), 1.905 (0.83), 1.943 (0.46), 2.057 (16.00), 2.323 (0.78), 2.327 (1.13), 2.331 (0.80), 2.367 (12.55), 2.518 (4.28), 2.523 (3.03), 2.608 (0.43), 2.634 (0.75), 2.640 (0.73), 2.660 (0.47), 2.665 (1.16), 2.669 (1.45), 2.673 (1.08), 2.982 (0.43), 2.991 (0.74), 3.000 (0.42), 3.154 (0.46), 3.181 (0.76), 3.214 (0.45), 3.973 (0.58), 4.008 (0.52), 4.597 (0.55), 4.630 (0.52), 5.768 (0.69), 5.786 (1.05), 5.803 (0.67), 7.106 (1.17), 7.241 (2.49), 7.279 (0.83), 7.299 (1.83), 7.318 (1.06), 7.377 (1.03), 7.491 (0.61), 7.509 (1.02), 7.526 (0.49), 7.650 (0.56), 7.668 (1.03), 7.686 (0.51), 8.637 (0.71), 8.643 (0.74), 8.655 (0.73), 8.689 (1.68), 8.693 (1.73), 8.873 (3.08), 8.878 (2.80).

### Example 45

### 2-methyl-N-{(1R)-1-[2-methyl-3-(trifluoromethyl)phenyl]ethyl}-6-(pyrimidin-5-yl)pyrido[2,3-d]pyrimidin-4-amine

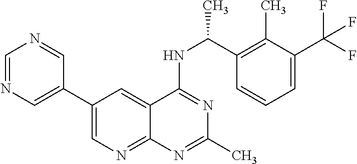 [SMILES: Cc1nc(N[C@H](C)c2cccc(C(F)(F)F)c2C)c2cc(-c3cncnc3)cnc2n1]

To a solution of 6-bromo-2-methyl-N-{(1R)-1-[2-methyl-3-(trifluoromethyl)phenyl]ethyl}-pyrido[2,3-d]pyrimidin-4-amine (Example 6, 50.0 mg, 118 μmol) and pyrimidin-5-ylboronic acid (39.3 mg, 317 μmol) in 1,4-dioxane (1.4 mL) was added aq. K₃PO₄ sol. (350 μL, 0.50 M, 180 μmol) and XPhosPdG2 (13.9 mg, 17.6 μmol) and the mixture was stirred at 100° C. overnight. The mixture was filtered, diluted with ethyl acetate and washed with water. The aq. phase was extracted with ethyl acetate, the combined org. phases were washed with brine, filtered through a hydrophobic filter, and concentrated under reduced pressure. Purification by HPLC (basic method) gave the titled compound (35.0 mg, 95% purity, 67% yield).

LC-MS (LC-MS METHOD 2): Rₜ=1.18 min; MS (ESIpos): m/z=425 [M+H]⁺

¹H NMR (400 MHz, DMSO-d₆) δ ppm 9.41 (d) 9.36 (s) 9.29 (d) 9.28 (s) 8.90 (d) 7.79 (d) 7.56 (d) 7.37 (t) 5.76 (quin) 2.63 (s) 2.52-2.52 (m) 2.42 (s) 1.60 (d)

### Example 46

### 1-{4-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-3,6-dihydropyridin-1(2H)-yl}ethan-1-one

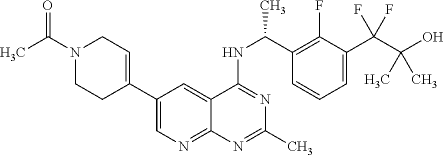 [SMILES: CC(=O)N1CC=C(c2cnc3nc(C)nc(N[C@H](C)c4cccc(C(F)(F)C(C)(C)O)c4F)c3c2)CC1]

tert-butyl 4-(4-{[(1R)-1-(3-{1,1-difluoro-2-methyl-2-[(triethylsilyl)oxy]propyl}-2-fluorophenyl)ethyl]-amino}-2-methylpyrido[2,3-d]pyrimidin-6-yl)-3,6-dihydropyridine-1(2H)-carboxylate (Intermediate 19, 188 mg, 274 μmol) and triethyl silane (4.4 μL, 27 μmol) were dissolved in dichloromethane (2.0 mL) and cooled to 0° C. TFA (320 μL, 4.1 mmol) was added dropwise. The mixture was allowed to warm to RT and was stirred overnight. Toluene was added and the solvent was evaporated. The residue was dissolved in dichloromethane (1.2 mL) and N,N-diisopropylethylamine (110 μL, 600 μmol) was added, followed by acetic anhydride (28 μL, 300 μmol) at RT. The mixture was stirred overnight. Toluene was added and the solvent was evaporated. Purification by HPLC (basic method) to give the titled compound (79.5 mg, 95% purity, 54% yield).

LC-MS (LC-MS METHOD 2): Rₜ=1.05 min; MS (ESIpos): m/z=514 [M+H]⁺

¹H NMR (400 MHz, DMSO-d₆) δ ppm 9.07-9.13 (m) 8.80-8.83 (m) 8.71-8.78 (m) 7.60 (br t) 7.31 (t) 7.22 (t) 6.45-6.51 (m) 5.80 (quin) 5.34 (s) 4.20 (br dd) 3.72 (dt) 3.38-3.45 (m) 3.21-3.30 (m) 2.98 (t) 2.67-2.75 (m) 2.52-2.64 (m) 2.33-2.40 (m) 2.06-2.12 (m) 1.60 (d) 1.22 (d)

### Example 47

### 1-{4-[4-({(1R)-1-[3-(1,1-difluoro-2-hydroxy-2-methylpropyl)-2-fluorophenyl]ethyl}amino)-2-methylpyrido[2,3-d]pyrimidin-6-yl]piperidin-1-yl}ethan-1-one

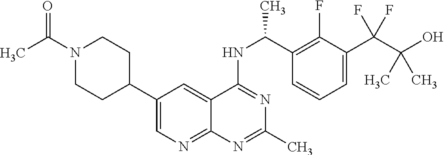 [SMILES: CC(=O)N1CCC(c2cnc3nc(C)nc(N[C@H](C)c4cccc(C(F)(F)C(C)(C)O)c4F)c3c2)CC1]

1-{4-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-3,6-dihydropyridin-1 (2H)-yl}ethan-1-one (Example 46, 58.0 mg, 113 μmol) was dissolved in EtOH (2.0 mL) and THF. Pd on carbon (12.0 mg, 10 wt %, 11.3 μmol) was added under argon. The atmosphere was exchanged to hydrogen gas and the mixture was stirred for 6 hours at RT. The mixture was diluted with dichloromethane and filtered. The solvent was evaporated, and the residue was purified by HPLC (basic method) to give the titled compound (13.5 mg, 22% yield).

LC-MS (LC-MS METHOD 2): Rₜ=0.97 min; MS (ESIpos): m/z=516 [M+H]⁺

¹H NMR (400 MHz, DMSO-d₆) δ ppm 8.87 (d) 8.70 (d) 8.64 (dd) 7.58 (t) 7.32 (t) 7.22 (t) 5.78 (quin) 5.34 (s) 4.59-4.65 (m) 3.96-4.02 (m) 3.15-3.23 (m) 2.99 (tt) 2.60-2.65 (m) 2.52-2.55 (m) 2.33-2.37 (m) 2.06 (s) 1.85-1.95 (m) 1.54-1.72 (m) 1.17-1.26 (m)

### Example 48

### 6-[2-methyl-4-({(1R)-1-[2-methyl-3-(trifluoromethyl)phenyl]ethyl}amino)pyrido[2,3-d]pyrimidin-6-yl]-2,6-diazaspiro[3.3]heptane-2-carboxamide

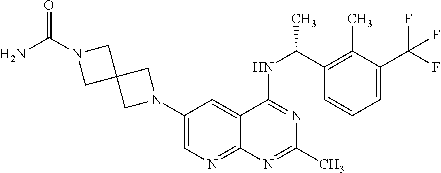 [SMILES: Cc1nc(N[C@H](C)c2cccc(C(F)(F)F)c2C)c2cc(N3CC4(CN(C(N)=O)C4)C3)cnc2n1]

Tert-butyl 6-[2-methyl-4-({(1R)-1-[2-methyl-3-(trifluoromethyl)phenyl]ethyl}amino)pyrido[2,3-d]pyrimidin-6-yl]-2,6-diazaspiro[3.3]heptane-2-carboxylate (Example 7, 60.0 mg, 111 μmol) and triethyl silane (1.8 μL, 11 μmol) were dissolved in dichloromethane (0.8 mL) and cooled to 0° C. TFA (130 μL, 1.7 mmol) was added dropwise. The mixture was allowed to warm to RT and was stirred for 7 hours. Toluene was added and the solvent was evaporated. N,N-diisopropylethylamine (42 μL, 240 μmol) was added to the residue, followed by trimethylsilyl isocyanate (16 μL, 0.122 mmol). The mixture was stirred at RT overnight. Toluene was added and the solvent was evaporated. The residue was purified by HPLC and preparative TLC using dichloromethane/MeOH 9:1 as eluent to give the titled compound (2.8 mg, 95% purity, 5% yield).

LC-MS (LC-MS METHOD 2): Rₜ=1.10 min; MS (ESIpos): m/z=486 [M+H]⁺

1H NMR (400 MHz, DMSO-d6) δ ppm 8.49 (d) 8.33 (d) 7.76 (d) 7.67 (d) 7.53 (d) 7.35 (t) 5.92 (s) 5.70 (quin) 4.11-4.16 (m) 4.00 (s) 2.60-2.68 (m) 2.52-2.55 (m) 2.29-2.36 (m) 1.56 (d) 1.17-1.30 (m)

### Example 49

### 1-{3-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-3,6-diazabicyclo[3.1.1]heptan-6-yl}ethan-1-one

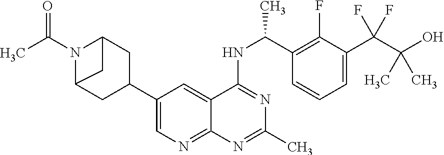 [SMILES: CC(=O)N1C2CC(c3cnc4nc(C)nc(N[C@H](C)c5cccc(C(F)(F)C(C)(C)O)c5F)c4c3)CC1C2]

tert-butyl 3-(4-{[(1R)-1-(3-{1,1-difluoro-2-methyl-2-[(triethylsilyl)oxy]propyl}-2-fluorophenyl)ethyl]-amino}-2-methylpyrido[2,3-d]pyrimidin-6-yl)-3,6-diazabicyclo[3.1.1]heptane-6-carboxylate (Intermediate 20, 59.0 mg, 84.2 μmol) and triethyl silane (1.3 μL, 8.4 μmol) were dissolved in dichloromethane (0.6 mL) and cooled to 0° C. TFA (97 μL, 1.3 mmol) was added dropwise and the mixture was allowed to warm to RT and was stirred overnight. Toluene (1 mL) was added, the solvent was evaporated, and the residue was redissolved in dichloromethane (0.6 mL). N,N-diisopropylethylamine (32 μL, 190 μmol) was added, followed by acetic anhydride (8.7 μL, 93 μmol). The mixture was stirred at RT overnight. The crude was purified by HPLC and by preparative TLC using dichloromethane/EtOH 9:1 as eluent to give the titled compound (16.2 mg, 95% purity, 35% yield).

LC-MS (LC-MS METHOD 2): Rₜ=0.98 min; MS (ESIpos): m/z=529 [M+H]⁺

¹H NMR (400 MHz, DMSO-d₆) δ ppm 8.67 (d) 8.39 (t) 7.77 (d) 7.57 (q) 7.27-7.33 (m) 7.17-7.24 (m) 5.74-5.84 (m) 5.34 (s) 4.71 (br s) 4.44-4.50 (m) 3.87-3.96 (m) 3.67-3.85 (m) 3.46-3.56 (m) 2.62-2.73 (m) 2.52-2.52 (m) 2.26-2.45 (m) 1.86 (d) 1.67 (d) 1.59 (d) 1.22 (br d)

### Example 50

### 1-{(1 S,4S)-5-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-2,5-diazabicyclo[2.2.1]heptan-2-yl}ethan-1-one

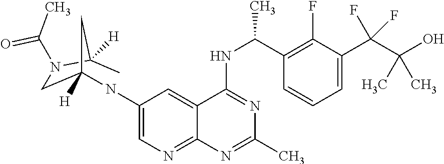

Tert-butyl (1S,4S)-5-(4-{[(1R)-1-(3-{1,1-difluoro-2-methyl-2-[(triethylsilyl)oxy]propyl}-2-fluorophenyl)-ethyl]amino}-2-methylpyrido[2,3-d]pyrimidin-6-yl)-2,5-diazabicyclo[2.2.1]heptane-2-carboxylate (Intermediate 21, 74.0 mg, 106 μmol) and triethyl silane (1.7 μL, 11 μmol) were dissolved in dichloromethane (0.7 mL) and cooled to 0° C. TFA (120 μL, 1.6 mmol) was added dropwise and the mixture was allowed to warm to RT and was stirred overnight. Toluene (1 mL) was added, and the solvent was evaporated. The residue was dissolved in dichloromethane (1.2 mL) at RT. N,N-diisopropylethylamine (40 μL, 230 μmol) was added, followed by acetic anhydride (11 μL, 120 μmol). The mixture was stirred overnight. Toluene was added and the solvent was evaporated. The crude was purified by HPLC and additionally by preparative TLC using dichloromethane/MeOH 1:1 as eluent to give the titled compound (13.9 mg, 95% purity, 24% yield).

LC-MS (LC-MS METHOD 2): Rₜ=0.96 min; MS (ESIpos): m/z=529 [M+H]⁺

¹H NMR (400 MHz, DMSO-d₆) δ ppm 8.61 (d) 8.57 (s) 8.24-8.32 (m) 7.74 (d) 7.71 (d) 7.57 (q) 7.27-7.33 (m) 7.21 (td) 5.73-5.81 (m) 5.34 (s) 4.89 (s) 4.84 (s) 4.76 (br d) 3.73 (dd) 3.59-3.67 (m) 3.37-3.46 (m) 3.29-3.30 (m) 3.18-3.29 (m) 2.67 (dt) 2.52-2.52 (m) 2.38-2.45 (m) 2.26-2.35 (m) 1.87-2.09 (m) 1.82 (s) 1.59 (dd) 1.22 (br d)

### Example 51

### 1-{(1 R,4R)-5-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-2,5-diazabicyclo[2.2.1]heptan-2-yl}ethan-1-one

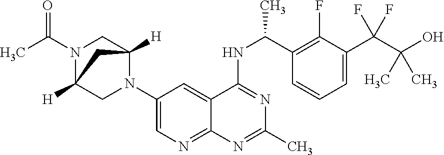 [SMILES: CC(=O)N1C[C@H]2C[C@@H]1CN2c1cnc2nc(C)nc(N[C@H](C)c3cccc(C(F)(F)C(C)(C)O)c3F)c2c1]

Tert-butyl (1 R,4R)-5-[4-({(1R)-1-[3-(1,1-difluoro-2-hydroxy-2-methylpropyl)-2-fluorophenyl]ethyl}-amino)-2-methylpyrido[2,3-d]pyrimidin-6-yl]-2,5-diazabicyclo[2.2.1]heptane-2-carboxylate (Intermediate 22, 43.0 mg, 73.3 μmol) and triethyl silane (1.2 μL, 7.3 μmol) were dissolved in dichloromethane and cooled to 0° C. TFA (85 μL, 1.1 mmol) was added dropwise. The mixture was allowed to warm to RT and was stirred overnight. Toluene (1 mL) was added, and the solvent was evaporated. The residue was dissolved in dichloromethane (1.2 mL) at RT. N,N-diisopropylethylamine (51 μL, 290 μmol) was added, followed by acetic anhydride (7.6 μL, 81 μmol). The mixture was stirred overnight. Toluene was added and the solvent was evaporated. The crude was purified by HPLC and additionally by preparative TLC using dichloromethane/MeOH 1:1 as eluent to give the titled compound (14.1 mg, 95% purity, 35% yield).

LC-MS (LC-MS METHOD 2): Rₜ=0.96 min; MS (ESIpos): m/z=529 [M+H]⁺

¹H NMR (400 MHz, DMSO-d₆) δ ppm 8.62 (d) 8.57 (d) 8.29 (br d) 8.25 (br d) 7.73 (br d) 7.70 (br d) 7.58 (br t) 7.31 (br t) 7.21 (t) 5.74-5.81 (m) 5.34 (s) 4.89 (s) 4.83 (s) 4.75 (br d) 3.73 (dd) 3.63 (td) 3.37-3.46 (m) 3.18-3.31 (m) 2.67 (dt) 2.52-2.55 (m) 2.28-2.34 (m) 1.93-2.08 (m) 1.82 (s) 1.58 (dd) 1.22 (br d)

### Example 52

### 1,1-difluoro-1-{2-fluoro-3-[(1R)-1-{[2-methyl-6-(4-methylpiperazin-1-yl)pyrido[2,3-d]pyrimidin-4-yl]amino}ethyl]phenyl}-2-methylpropan-2-ol

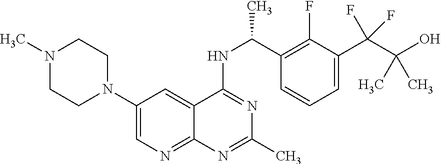 [SMILES: Cc1nc(N[C@H](C)c2cccc(C(F)(F)C(C)(C)O)c2F)c2cc(N3CCN(C)CC3)cnc2n1]

N-[(1R)-1-(3-{1,1-difluoro-2-methyl-2-[(triethylsilyl)oxy]propyl}-2-fluorophenyl)ethyl]-2-methyl-6-(4-methylpiperazin-1-yl)pyrido[2,3-d]pyrimidin-4-amine (Intermediate 23, 34.0 mg, 56.4 μmol) and triethyl silane (0.90 μL, 5.6 μmol) were dissolved in dichloromethane(0.4 mL) and cooled to 0° C. TFA (65 μL, 850 μmol) was added dropwise. The mixture was allowed to warm to RT and was stirred overnight. Toluene was added and the solvent was evaporated. The residue was purified by HPLC purification to give the titled compound (13.0 mg, 95% purity, 45% yield).

LC-MS (LC-MS METHOD 2): Rₜ=1.05 min; MS (ESIpos): m/z=489 [M+H]⁺

¹H NMR (400 MHz, DMSO-d₆) δ ppm 8.86 (d) 8.43 (d) 8.04 (d) 7.58 (t) 7.31 (t) 7.21 (t) 5.75-5.82 (m) 5.34 (s) 3.27-3.32 (m) 2.52-2.59 (m) 2.32-2.34 (m) 2.31 (s) 2.26 (s) 1.58 (d) 1.23 (s) 1.20 (s)

### Example 53

### 1-{4-[4-({(1R)-1-[3-(1,1-difluoro-2-hydroxy-2-methylpropyl)-2-fluorophenyl]ethyl}amino)-2-methylpyrido[2,3-d]pyrimidin-6-yl]piperazin-1-yl}ethan-1-one

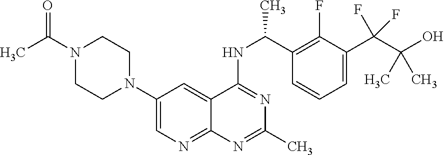 [SMILES: CC(=O)N1CCN(c2cnc3nc(C)nc(N[C@H](C)c4cccc(C(F)(F)C(C)(C)O)c4F)c3c2)CC1]

1-[4-(4-{[(1R)-1-(3-{1,1-difluoro-2-methyl-2-[(triethylsilyl)oxy]propyl}-2-fluorophenyl)ethyl]amino}-2-methylpyrido[2,3-d]pyrimidin-6-yl)piperazin-1-yl]ethan-1-one (Intermediate 24, 45.0 mg, 71.3 μmol) and triethyl silane (1.1 μL, 7.1 μmol) were dissolved in dichloromethane (0.7 mL) and cooled to 0° C. TFA (82 μL, 1.1 mmol) was added dropwise. The mixture was allowed to warm to RT and was stirred overnight. Toluene was added and the solvent was evaporated. The residue was purified by HPLC to give the titled compound (27.5 mg, 95% purity, 71% yield).

LC-MS (LC-MS METHOD 2): Rₜ=0.98 min; MS (ESIpos): m/z=517 [M+H]⁺

¹H NMR (400 MHz, DMSO-d₆) δ ppm 8.89 (d) 8.45 (d) 8.08 (d) 7.58 (t) 7.31 (t) 7.21 (t) 5.79 (quin) 5.34 (s) 3.66 (q) 3.37-3.42 (m) 3.23-3.30 (m) 2.67 (dt) 2.52-2.55 (m) 2.33-2.34 (m) 2.32 (s) 2.08 (s) 1.59 (d) 1.23 (s) 1.20 (s)

### Example 54

### 1-{6-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-2,6-diazaspiro[3.3]heptan-2-yl}ethan-1-one

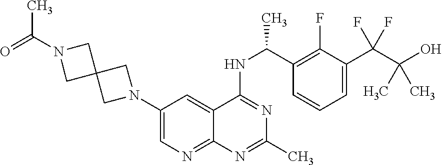 [SMILES: CC(=O)N1CC2(C1)CN(c1cnc3nc(C)nc(N[C@H](C)c4cccc(C(F)(F)C(C)(C)O)c4F)c3c1)C2]

Tert-butyl 6-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-2,6-diazaspiro[3.3]heptane-2-carboxylate (Intermediate 25, 59.0 mg, 101 μmol) and triethyl silane (1.6 μL, 10 μmol) were dissolved in dichloromethane (0.75 mL) and cooled to 0° C. TFA (120 μL, 1.5 mmol) was added dropwise. The mixture was allowed to warm to RT and was stirred overnight. Toluene (1 mL) was added, and the solvent was evaporated. The residue was dissolved in dichloromethane (1.2 mL) at RT. N,N-diisopropylethylamine (39 μL, 220 μmol) was added, followed by acetic anhydride (10 μL, 110 μmol). The mixture was stirred for 3 days at RT. Toluene was added and the solvent was evaporated. The crude was purified by HPLC to give the titled compound (22.9 mg, 95% purity, 41% yield).

LC-MS (LC-MS METHOD 2): Rₜ=0.98 min; MS (ESIpos): m/z=529 [M+H]⁺

¹H NMR (400 MHz, DMSO-d₆) δ ppm 8.39 (d) 8.36 (d) 7.70 (d) 7.56 (t) 7.30 (t) 7.20 (t) 5.77 (quin) 5.34 (s) 4.34 (s) 4.11-4.18 (m) 4.06 (s) 2.52-2.55 (m) 2.27-2.46 (m) 1.77 (s) 1.58 (d) 1.17-1.26 (m) 1.22 (d)

### Example 55

### N-{(3R)-1-[4-({(1R)-1-[3-(1,1-difluoro-2-hydroxy-2-methylpropyl)-2-fluorophenyl]ethyl}amino)-2-methylpyrido[2,3-d]pyrimidin-6-yl]pyrrolidin-3-yl}acetamide

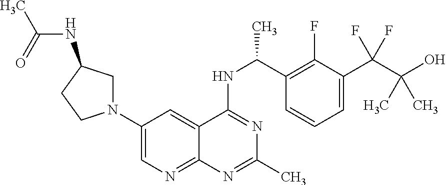 [SMILES: CC(=O)N[C@@H]1CCN(c2cnc3nc(C)nc(N[C@H](C)c4cccc(C(F)(F)C(C)(C)O)c4F)c3c2)C1]

1-(3-{(1R)-1-[(6-bromo-2-methylpyrido[2,3-d]pyrimidin-4-yl)amino]ethyl}-2-fluorophenyl)-1,1-difluoro-2-methylpropan-2-ol (Intermediate 18, 50.0 mg, 107 μmol) and N-[(3R)-pyrrolidin-3-yl]acetamide (16.4 mg, 128 μmol) were dissolved in dioxane (1.0 mL). Sodium tert-butoxide (13.3 mg, 139 μmol) and XPhos (10.2 mg, 21.3 μmol) were added, the atmosphere was exchanged to argon and Pd₂dba₃ (9.76 mg, 10.7 μmol) was added. The mixture was heated to 100° C. overnight. The mixture was cooled to RT and sat. brine and ethyl acetate were added. The aq. phase was extracted with ethyl acetate. The organic phase was dried, and the solvent was evaporated. The residue was purified by HPLC and additionally by preparative TLC using dichloromethane/MeOH 9:1 as eluent to give the titled compound (13.0 mg, 95% purity, 24% yield).

LC-MS (LC-MS METHOD 2): Rₜ=0.99 min; MS (ESIpos): m/z=517 [M+H]⁺

¹H NMR (400 MHz, DMSO-d₆) δ ppm 8.51 (d) 8.34 (d) 8.22 (d) 7.63 (d) 7.56 (t) 7.30 (t) 7.21 (t) 5.75-5.83 (m) 5.34 (s) 4.40-4.47 (m) 3.53-3.67 (m) 3.39-3.49 (m) 3.21-3.30 (m) 2.52-2.56 (m) 2.19-2.34 (m) 1.92-2.01 (m) 1.83 (s) 1.58 (d) 1.27 (br d) 1.22 (d)

### Example 56

### 1-{4-[4-({(1R)-1-[3-(difluoromethyl)-2-fluorophenyl]ethyl}amino)-2,7-dimethylpyrido[2,3-d]pyrimidin-6-yl]-3,6-dihydropyridin-1(2H)-yl}ethan-1-one

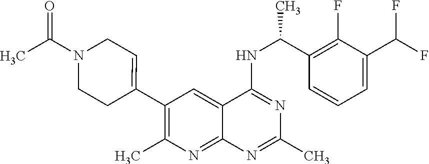 [SMILES: CC(=O)N1CC=C(c2cc3c(N[C@H](C)c4cccc(C(F)F)c4F)nc(C)nc3nc2C)CC1]

6-Bromo-N-{(1R)-1-[3-(difluoromethyl)-2-fluorophenyl]ethyl}-2,7-dimethylpyrido[2,3-d]pyrimidin-4-amine (Intermediate 6, 167 mg, 393 μmol), 1-[4-(4,4,5,5-tetramethyl-1,3,2-dioxaborolan-2-yl)-3,6-dihydropyridin-1(2H)-yl]ethan-1-one (118 mg, 471 μmol) and bis(triphenylphosphine)palladium(II) dichloride (27.6 mg, 0.039 mmol) were dissolved in DME (1.7 mL) and EtOH (1.7 mL). Potassium carbonate (81.4 mg, 589 μmol) was added, and the atmosphere was exchanged to argon. The mixture was heated to 100° C. for 6 h in a microwave. The mixture was cooled to RT and ethyl acetate and sat. brine was added. The aq. phase was extracted with ethyl acetate and the organic phase was dried. The solvent was evaporated, and the residue was purified by flash column chromatography on silica to give the titled compound (131 mg, 90% purity, 64% yield).

LC-MS (LC-MS METHOD 2): Rₜ=1.07 min; MS (ESIneg): m/z=468 [M−H]⁻

¹H-NMR (400 MHz, DMSO-d6) δ[ppm]: 0.797 (0.48), 0.814 (0.52), 0.821 (0.53), 0.903 (0.60), 1.066 (0.76), 1.572 (3.77), 1.589 (3.80), 2.075 (6.37), 2.102 (8.20), 2.332 (0.46), 2.359 (16.00), 2.387 (0.70), 2.392 (0.75), 2.518 (2.64), 2.523 (1.84), 2.560 (6.29), 2.673 (0.41), 3.672 (0.86), 3.686 (1.74), 3.701 (1.15), 3.717 (0.84), 3.721 (0.81), 3.731 (0.41), 3.736 (0.41), 4.121 (1.34), 4.127 (1.36), 4.164 (1.05), 4.171 (1.05), 5.758 (2.16), 5.777 (1.16), 5.795 (0.75), 5.820 (1.55), 7.099 (1.14), 7.235 (2.35), 7.271 (0.79), 7.290 (1.73), 7.309 (0.99), 7.371 (1.00), 7.485 (0.64), 7.502 (1.07), 7.520 (0.52), 7.651 (0.58), 7.668 (1.06), 7.687 (0.53), 8.533 (2.39), 8.543 (1.82), 8.622 (0.82), 8.640 (0.80).

### Example 57

### 1-{4-[4-({(1R)-1-[3-(difluoromethyl)-2-fluorophenyl]ethyl}amino)-2,7-dimethylpyrido[2,3-d]pyrimidin-6-yl]piperidin-1-yl}ethan-1-one

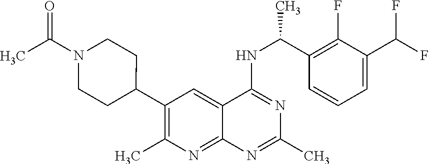 [SMILES: CC(=O)N1CCC(c2cc3c(N[C@H](C)c4cccc(C(F)F)c4F)nc(C)nc3nc2C)CC1]

1-{4-[4-({(1R)-1-[3-(difluoromethyl)-2-fluorophenyl]ethyl}amino)-2,7-dimethylpyrido[2,3-d]pyrimidin-6-yl]-3,6-dihydropyridin-1(2H)-yl}ethan-1-one (Example 56, 51.0 mg, 109 μmol) was dissolved in EtOH (2.3 mL) and THE under argon. Pd on carbon (11.6 mg, 10 wt %, 10.9 μmol) was added and the atmosphere was exchanged to hydrogen and the mixture was stirred for 4 hours. The mixture was diluted with dichloromethane and then filtered. The solvent was evaporated, and the residue was purified by HPLC to give the titled compound (28.4 mg, 95% purity, 53% yield).

LC-MS (LC-MS METHOD 2): Rₜ=1.08 min; MS (ESIneg): m/z=470 [M−H]⁻

¹H-NMR (400 MHz, DMSO-d6) δ[ppm]: 1.594 (4.04), 1.612 (4.07), 1.633 (0.46), 1.644 (0.41), 1.653 (0.59), 1.684 (0.53), 1.807 (0.60), 1.839 (0.44), 1.862 (0.57), 1.900 (0.45), 2.062 (14.73), 2.322 (0.70), 2.333 (9.23), 2.335 (9.43), 2.518 (2.31), 2.523 (1.54), 2.638 (0.47), 2.663 (16.00), 2.696 (0.40), 3.100 (0.59), 3.192 (0.44), 3.220 (0.77), 3.252 (0.45), 3.347 (0.46), 3.974 (0.59), 4.008 (0.54), 4.619 (0.57), 4.651 (0.54), 5.757 (0.61), 5.775 (0.92), 5.792 (0.59), 7.102 (1.23), 7.238 (2.62), 7.276 (0.89), 7.295 (1.93), 7.314 (1.10), 7.374 (1.07), 7.483 (0.61), 7.501 (1.03), 7.518 (0.50), 7.627 (0.59), 7.645 (1.06), 7.663 (0.52), 8.557 (3.29), 8.571 (0.83), 8.577 (0.77), 8.588 (0.63).

### Example 58

### 2-[2,7-dimethyl-4-({(1R)-1-[2-methyl-3-(trifluoromethyl)phenyl]ethyl}amino)pyrido[2,3-d]pyrimidin-6-yl]-1 lambda⁶,2-thiazolidine-1,1-dione

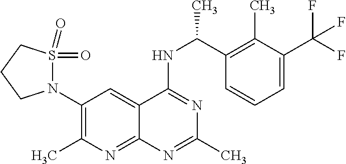 [SMILES: Cc1nc(N[C@H](C)c2cccc(C(F)(F)F)c2C)c2cc(N3CCCS3(=O)=O)c(C)nc2n1]

6-Bromo-2,7-dimethyl-N-{(1R)-1-[2-methyl-3-(trifluoromethyl)phenyl]ethyl}pyrido[2,3-d]pyrimidin-4-amine (Intermediate 5, 50.0 mg, 114 μmol) and 1lambda⁶,2-thiazolidine-1,1-dione (13.8 mg, 114 μmol) were dissolved in dioxane (1.0 mL). Potassium phosphate (48.3 mg, 228 μmol), N,N′-dimethyl ethylenediamine (10 μL, 91 μmol) and copper(I)iodide (8.67 mg, 45.5 μmol) were added, the atmosphere was exchanged to argon and the mixture was heated to 100° C. overnight. The mixture was cooled to RT, dichloromethane was added and then filtered. The solvent was evaporated, and the residue was purified by HPLC to give the titled compound (12.0 mg, 95% purity, 21% yield).

LC-MS (LC-MS METHOD 2): Rₜ=1.21 min; MS (ESIpos): m/z=480 [M+H]⁺

¹H-NMR (400 MHz, DMSO-d6) δ[ppm]: 1.232 (0.99), 1.559 (4.25), 1.577 (4.25), 2.085 (0.47), 2.327 (1.87), 2.331 (1.33), 2.366 (13.79), 2.518 (9.33), 2.523 (6.01), 2.620 (16.00), 2.669 (1.93), 2.673 (1.35), 3.501 (1.82), 3.520 (2.96), 3.539 (1.52), 3.745 (0.89), 3.753 (0.88), 3.763 (1.63), 3.769 (1.58), 3.779 (0.84), 3.786 (0.82), 5.699 (0.66), 5.716 (1.03), 5.733 (0.65), 5.760 (4.49), 7.351 (0.62), 7.370 (1.32), 7.390 (0.78), 7.543 (1.46), 7.562 (1.16), 7.743 (1.30), 7.762 (1.15), 8.852 (4.18), 8.866 (1.21), 8.883 (1.13).

### Example 59

### 2-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]-1 lambda⁶,2-thiazolidine-1,1-dione

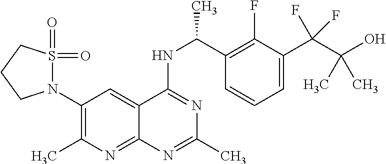 [SMILES: Cc1nc(N[C@H](C)c2cccc(C(F)(F)C(C)(C)O)c2F)c2cc(N3CCCS3(=O)=O)c(C)nc2n1]

2-(4-{[(1R)-1-(3-{1,1-difluoro-2-methyl-2-[(triethylsilyl)oxy]propyl}-2-fluorophenyl)ethyl]amino}-2,7-dimethylpyrido[2,3-d]pyrimidin-6-yl)-1 lambda6,2-thiazolidine-1,1-dione (Intermediate 26, 70.0 mg, 110 μmol) and triethyl silane (1.8 μL, 11 μmol) were dissolved in dichloromethane (0.87 mL) and cooled to 0° C. TFA (170 μL, 2.2 mmol) was added dropwise. The mixture was allowed to warm to RT and was stirred for 3 days. Toluene was added and the solvent was evaporated. The crude was purified by HPLC and additionally by preparative TLC using dichloromethane/MeOH 1:1 as eluent to give the titled compound (2.0 mg, 95% purity, 3% yield).

LC-MS (LC-MS METHOD 2): Rₜ=1.01 min; MS (ESIpos): m/z=524 [M+H]⁺

¹H-NMR (400 MHz, DMSO-d6) δ[ppm]: 1.154 (1.96), 1.172 (4.07), 1.190 (2.35), 1.203 (2.80), 1.229 (4.03), 1.582 (1.92), 1.599 (1.90), 1.987 (7.86), 2.084 (0.65), 2.327 (0.49), 2.359 (6.05), 2.518 (2.79), 2.523 (1.53), 2.629 (5.70), 2.669 (0.47), 3.159 (15.59), 3.171 (16.00), 3.499 (0.98), 3.504 (1.02), 3.518 (1.37), 3.537 (0.77), 3.756 (0.51), 3.773 (0.96), 3.792 (0.48), 3.999 (0.60), 4.017 (1.80), 4.035 (1.78), 4.053 (0.60), 4.089 (1.15), 4.102 (3.19), 4.115 (3.13), 4.128 (1.08), 5.342 (0.53), 5.758 (9.79), 5.779 (0.49), 7.224 (0.72), 7.243 (0.47), 7.320 (0.47), 7.589 (0.46), 8.803 (0.55), 8.822 (0.52), 8.883 (1.84).

### Example 60

### 1-{6-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]-2,6-diazaspiro[3.3]heptan-2-yl}ethan-1-one

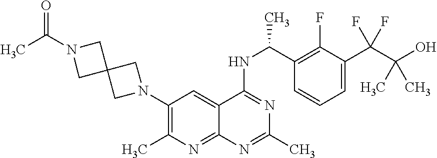 [SMILES: CC(=O)N1CC2(C1)CN(c1cc3c(N[C@H](C)c4cccc(C(F)(F)C(C)(C)O)c4F)nc(C)nc3nc1C)C2]

To a solution of intermediate 27 (1-[6-(4-{[(1R)-1-(3-{1,1-difluoro-2-methyl-2-[(triethylsilyl)oxy]propyl}-2-fluorophenyl)ethyl]amino}-2,7-dimethylpyrido[2,3-d]pyrimidin-6-yl)-2,6-diazaspiro[3.3]heptan-2-yl]ethan-1-one, 47.0 mg, 71.6 μmol) and triethyl silane (1.1 μl, 7.2 μmol) in dichloromethane (600 μl) was added trifluoroacetic acid (83 μl, 1.1 mmol) and the mixture was stirred at room temperature overnight. The mixture was then triturated with toluene and concentrated under reduced pressure. The obtained crude product was purified by preparative HPLC (basic method) to yield the titled compound (17.4 mg, 95% purity, 43% yield).

LC-MS (LC-MS METHOD 2): Rₜ=0.97 min; MS (ESIpos): m/z=543 [M+H]⁺

¹H NMR (400 MHz, DMSO-d₆) δ ppm 8.37 (d) 7.64 (s) 7.57 (t) 7.30 (t) 7.21 (t) 5.78 (quin) 5.34 (s) 4.33 (s) 4.11-4.17 (m) 4.05 (s) 2.52-2.56 (m) 2.43-2.47 (m) 2.29 (s) 1.77 (s) 1.58 (d) 1.17-1.27 (m).

### Example 61

### 4-acetyl-1-[2-methyl-4-({(1R)-1-[2-methyl-3-(trifluoromethyl)phenyl]ethyl}amino)pyrido[2,3-d]pyrimidin-6-yl]piperazin-2-one

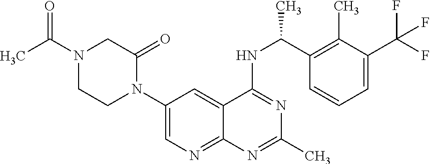 [SMILES: CC(=O)N1CCN(c2cnc3nc(C)nc(N[C@H](C)c4cccc(C(F)(F)F)c4C)c3c2)C(=O)C1]

Using the method described for Example 24, Intermediate 28 (tert-butyl 4-[2-methyl-4-({(1R)-1-[2-methyl-3-(trifluoromethyl)phenyl]ethyl}amino)pyrido[2,3-d]pyrimidin-6-yl]-3-oxopiperazine-1-carboxylate, 109 mg, 200 μmol), triethyl silane (not used for this example), trifluoroacetic acid (200 μl, 1.3 mmol), acetic anhydride (21 μl, 220 μmol) and N,N-diisopropylethylamine (77 μl, 440 μmol) gave the titled compound, 28.4 mg (95% purity, 28% yield) after preparative HPLC (basic method).

LC-MS (LC-MS METHOD 2): Rₜ=1.10 min; MS (ESIpos): m/z=487 [M+H]⁺

¹H-NMR (400 MHz, DMSO-d6) δ[ppm]: 1.550 (5.14), 1.568 (5.10), 2.090 (6.15), 2.121 (8.14), 2.390 (16.00), 2.518 (2.80), 2.523 (1.85), 2.616 (7.13), 3.312 (0.72), 3.391 (0.69), 3.399 (0.64), 3.819 (0.49), 3.841 (1.17), 3.853 (1.05), 3.873 (0.72), 3.884 (0.75), 3.908 (1.39), 3.917 (2.39), 3.928 (2.36), 3.946 (0.72), 4.257 (3.91), 4.360 (3.22), 5.708 (0.81), 5.725 (1.23), 5.742 (0.77), 7.338 (0.78), 7.357 (1.71), 7.377 (0.98), 7.542 (1.91), 7.561 (1.52), 7.746 (1.68), 7.765 (1.48), 8.791 (3.32), 8.798 (3.84), 8.814 (0.81), 8.939 (1.74), 8.946 (1.70), 8.955 (1.40), 8.962 (1.21).

### Example 62

### 1-{4-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]-5-methyl-3,6-dihydropyridin-1(2H)-yl}ethan-1-one

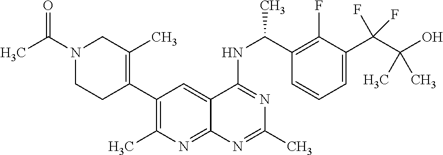 [SMILES: CC(=O)N1CCC(c2cc3c(N[C@H](C)c4cccc(C(F)(F)C(C)(C)O)c4F)nc(C)nc3nc2C)=C(C)C1]

Using the method described for Example 24, Intermediate 29 (tert-butyl 4-(4-{[(1R)-1-(3-{1,1-difluoro-2-methyl-2-[(triethylsilyl)oxy]propyl}-2-fluorophenyl)ethyl]amino}-2,7-dimethylpyrido[2,3-d]pyrimidin-6-yl)-5-methyl-3,6-dihydropyridine-1(2H)-carboxylate, 39.5 mg, 55.3 μmol), triethyl silane (0.88 μl, 5.5 μmol), trifluoroacetic acid (64 μl, 830 μmol), acetic anhydride (5.7 μl, 61 μmol) and N,N-diisopropylethylamine (21 μl, 120 μmol) gave the titled compound, 19.7 mg (95% purity, 62% yield) after preparative HPLC (basic method).

LC-MS (LC-MS METHOD 2): Rₜ=1.06 min; MS (ESIpos): m/z=542 [M+H]⁺

¹H-NMR (400 MHz, DMSO-d6) δ[ppm]: 1.197 (7.61), 1.223 (6.30), 1.433 (3.67), 1.539 (5.48), 1.556 (5.50), 2.091 (14.10), 2.263 (0.88), 2.323 (1.18), 2.327 (1.72), 2.343 (9.68), 2.347 (9.68), 2.454 (16.00), 2.518 (5.62), 2.523 (3.66), 2.665 (0.80), 2.669 (1.08), 2.673 (0.78), 3.298 (0.49), 3.548 (0.53), 3.566 (0.44), 3.582 (0.41), 3.589 (0.41), 3.601 (0.45), 3.730 (0.53), 3.740 (0.54), 3.839 (0.67), 3.850 (0.66), 3.882 (0.75), 4.040 (1.44), 4.127 (0.44), 4.144 (0.44), 5.334 (3.01), 5.340 (4.27), 5.739 (0.71), 5.757 (1.03), 5.770 (0.69), 7.183 (0.56), 7.203 (1.29), 7.215 (1.30), 7.221 (0.92), 7.234 (0.77), 7.288 (1.02), 7.305 (1.49), 7.324 (0.66), 7.550 (0.70), 7.572 (1.32), 7.590 (1.23), 7.606 (0.56), 7.621 (0.41), 8.472 (2.00), 8.486 (2.95), 8.532 (0.92), 8.551 (1.35), 8.563 (0.72).

### Example 63

### 1-{6-[2,7-dimethyl-4-({(1R)-1-[2-methyl-3-(trifluoromethyl)phenyl]ethyl}amino)pyrido[2,3-d]pyrimidin-6-yl]-2,6-diazaspiro[3.3]heptan-2-yl}ethan-1-one

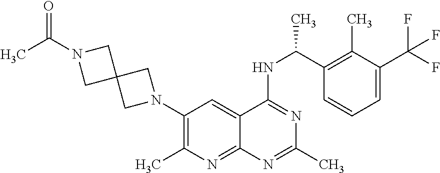 [SMILES: CC(=O)N1CC2(C1)CN(c1cc3c(N[C@H](C)c4cccc(C(F)(F)F)c4C)nc(C)nc3nc1C)C2]

Using the method described for Example 7, Intermediate 5 (6-bromo-2,7-dimethyl-N-{(1R)-1-[2-methyl-3-(trifluoromethyl)phenyl]ethyl}pyrido[2,3-d]pyrimidin-4-amine, 75.0 mg, 171 μmol), oxalic acid/1-(2,6-diazaspiro[3.3]heptan-2-yl)ethan-1-one (½) (94.9 mg, 256 μmol), sodium tert-butoxide (65.6 mg, 683 μmol), XPhos (16.3 mg, 34.1 μmol), Pd₂(dba)₃ (13.4 mg, 17.1 μmol) in 1,4-dioxane (1.9 ml) gave the titled compound (12.0 mg, 90% purity, 13% yield) after preparative TLC using dichloromethane/methanol (9:1) as eluent.

LC-MS (LC-MS METHOD 2): Rₜ=1.16 min; MS (ESIpos): m/z=499 [M+H]⁺

¹H NMR (400 MHz, DMSO-d₆) δ ppm 8.47 (d) 7.76-7.80 (m) 7.75 (s) 7.63 (s) 7.54 (d) 7.35 (t) 5.67-5.76 (m) 4.33 (s) 4.07-4.17 (m) 4.05 (s) 2.61 (s) 2.52-2.55 (m) 2.44-2.46 (m) 2.30 (s) 1.77 (s) 1.56 (d) 1.34 (d) 1.23 (s).

### Example 64

### 1-{(1 S,4S)-5-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]-2,5-diazabicyclo[2.2.1]heptan-2-yl}ethan-1-one

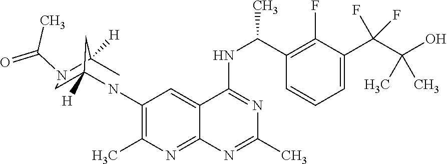

Using the method described for Example 24, Intermediate 31 (tert-butyl (1S,4S)-5-(4-{[(1R)-1-(3-{1,1-difluoro-2-methyl-2-[(triethylsilyl)oxy]propyl}-2-fluorophenyl)ethyl]amino}-2,7-dimethylpyrido[2,3-d]pyrimidin-6-yl)-2,5-diazabicyclo[2.2.1]heptane-2-carboxylate, 27.0 mg, 37.8 μmol), was treated with triethyl silane (0.60 μl, 3.8 μmol) and trifluoroacetic acid (44 μl, 570 μmol) in dichloromethane (400 μl) at room temperature overnight. The mixture was triturated with toluene and concentrated. The treatment was repeated with triethyl silane (0.60 μl, 3.8 μmol) and trifluoroacetic acid (44 μl, 570 μmol) in dichloromethane (400 al) at room temperature overnight, before the mixture was again triturated with toluene, concentrated and then treated with acetic anhydride (3.9 μl, 42 μmol) and N,N-diisopropylethylamine (14 μl, 83 μmol) according to the procedure described for Example 24 to give the titled compound (5.30 mg (95% purity, 25% yield) after preparative HPLC (basic method).

LC-MS (LC-MS METHOD 2): Rₜ=0.97 min; MS (ESIpos): m/z=543 [M+H]⁺

¹H NMR (400 MHz, DMSO-d₆) δ ppm 8.36 (t) 7.87 (s) 7.55 (t) 7.31 (t) 7.20 (t) 5.78 (quin) 5.34 (s) 4.64 (s) 4.44 (s) 3.72 (dd) 3.55-3.62 (m) 3.38-3.42 (m) 3.25-3.30 (m) 2.52-2.57 (m) 2.29 (d) 1.99-2.04 (m) 1.87 (s) 1.60 (dd) 1.22 (br d).

### Example 65

### 1-{4-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]-3,6-dihydropyridin-1(2H)-yl}ethan-1-one

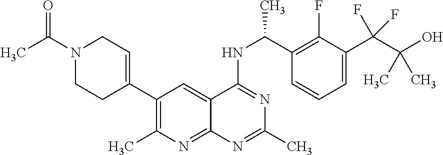 [SMILES: CC(=O)N1CC=C(c2cc3c(N[C@H](C)c4cccc(C(F)(F)C(C)(C)O)c4F)nc(C)nc3nc2C)CC1]

Using the method described for Example 24, Intermediate 32 (tert-butyl 4-(4-{[(1R)-1-(3-{1,1-difluoro-2-methyl-2-[(triethylsilyl)oxy]propyl}-2-fluorophenyl)ethyl]amino}-2,7-dimethylpyrido[2,3-d]pyrimidin-6-yl)-3,6-dihydropyridine-1(2H)-carboxylate, 116 mg, 166 μmol), triethylsilane (2.6 μl, 17 μmol), trifluoroacetic acid (190 μl, 2.5 mmol), acetic anhydride (17 μl, 180 μmol) and N,N-diisopropylethylamine (64 μl, 360 μmol) gave the titled compound (44.1 mg, 95% purity, 48% yield) after preparative TLC using dichloromethane/ethanol (9:1) as eluent.

LC-MS (LC-MS METHOD 2): Rₜ=1.02 min; MS (ESIpos): m/z=528 [M+H]⁺

¹H NMR (400 MHz, DMSO-d₆) δ ppm 8.60 (d) 8.54 (d) 7.58 (br t) 7.31 (br t) 7.21 (t) 5.73-5.83 (m) 5.34 (s) 4.15 (br dd) 3.66-3.74 (m) 2.58-2.68 (m) 2.54-2.57 (m) 2.37-2.42 (m) 2.34 (s) 2.32-2.34 (m) 2.09 (d) 1.55 (d) 1.21 (br d).

### Example 66

### 1-{4-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]piperidin-1-yl}ethan-1-one

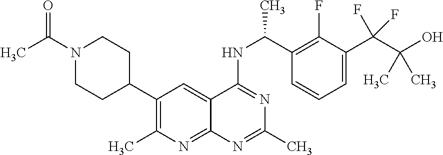 [SMILES: CC(=O)N1CCC(c2cc3c(N[C@H](C)c4cccc(C(F)(F)C(C)(C)O)c4F)nc(C)nc3nc2C)CC1]

Using the method described for Example 22, Intermediate 32 (1-{4-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]-3,6-dihydropyridin-1(2H)-yl}ethan-1-one, 24.8 mg, 47.0 μmol) in EtOH (1 ml) was hydrogenated with Pd/C (5.00 mg, 10% purity, 4.70 μmol) and H₂ atmosphere at rt for 6 h. The crude product was purified by prep. HPLC (basic method) to obtain the titled compound (16.2 mg, 95% purity, 62% yield) after preparative HPLC (basic method).

LC-MS (LC-MS METHOD 2): Rₜ=1.00 min; MS (ESIpos): m/z=530 [M+H]⁺

¹H NMR (400 MHz, DMSO-d₆) δ ppm 8.53-8.60 (m) 7.56 (t) 7.31 (t) 7.22 (t) 5.73-5.81 (m) 5.30-5.40 (m) 4.64 (br d) 3.99 (br d) 3.18-3.30 (m) 3.06-3.14 (m) 2.63-2.70 (m) 2.52-2.56 (m) 2.31-2.34 (m) 2.06 (s) 1.79-1.90 (m) 1.54-1.75 (m) 1.23 (s) 1.20 (s).

### Example 67

### 1-{4-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]piperazin-1-yl}ethan-1-one

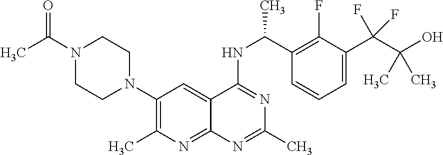 [SMILES: CC(=O)N1CCN(c2cc3c(N[C@H](C)c4cccc(C(F)(F)C(C)(C)O)c4F)nc(C)nc3nc2C)CC1]

To a solution of Intermediate 30 (1-[4-(4-{[(1R)-1-(3-{1,1-difluoro-2-methyl-2-[(triethylsilyl)oxy]propyl}-2-fluorophenyl)ethyl]amino}-2,7-dimethylpyrido[2,3-d]pyrimidin-6-yl)piperazin-1-yl]ethan-1-one, 30.0 mg, 46.5 μmol) and triethyl silane (0.74 μl, 4.7 μmol) in dichloromethane (0.4 ml) was added trifluoroacetic acid (54 μl, 700 μmol), and the mixture was stirred overnight at room temperature. Toluene was then added, and the mixture was concentrated. The residue was purified by HPLC (basic method) to give the titled compound (14.8 mg, 95% purity, 57% yield).

LC-MS (LC-MS METHOD 2): Rₜ=0.99 min; MS (ESIpos): m/z=531 [M+H]⁺

¹H NMR (400 MHz, DMSO-d₆) δ ppm 8.47 (d) 8.31 (s) 7.57 (t) 7.31 (t) 7.21 (t) 5.77 (quin) 5.34 (s) 3.60-3.70 (m) 2.89-3.01 (m) 2.67 (dt) 2.60 (s) 2.52-2.59 (m) 2.33-2.35 (m) 2.32 (s) 2.08 (s) 1.58 (d) 1.21 (d) 1.17-1.28 (m).

### Example 68

### 4-acetyl-1-[2,7-dimethyl-4-({(1R)-1-[2-methyl-3-(trifluoromethyl)phenyl]ethyl}amino)pyrido[2,3-d]pyrimidin-6-yl]piperazin-2-one

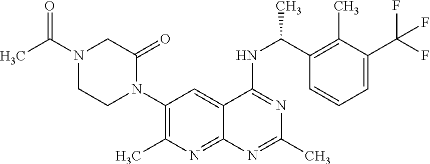 [SMILES: CC(=O)N1CCN(c2cc3c(N[C@H](C)c4cccc(C(F)(F)F)c4C)nc(C)nc3nc2C)C(=O)C1]

Using the method described for Intermediate 28, Intermediate 5 (6-bromo-2,7-dimethylpyrido[2,3-d]pyrimidin-4-ol, 75 mg, 171 μmol), tert-butyl-oxopiperazine-1-carboxylate (34 mg, 171 μmol), copper iodide (13 mg, 68 μmol), and potassium phosphate (72 mg, 341 μmol) in DMF (0.8 ml) gave tert-butyl 6-[4-({(1R)-1-[3-(difluoromethyl)-2-fluorophenyl]ethyl}amino)-2-methyl-7-(trifluoromethyl)pyrido[2,3-d]pyrimidin-6-yl]-2,6-diazaspiro[3.3]heptane-2-carboxylate (100 mg), which was used directly for the following step

Using the method described for Example 24, the crude product from the previous step (tert-butyl 6-[4-({(1R)-1-[3-(difluoromethyl)-2-fluorophenyl]ethyl}amino)-2-methyl-7-(trifluoromethyl)pyrido[2,3-d]pyrimidin-6-yl]-2,6-diazaspiro[3.3]heptane-2-carboxylate, 196 mg, 350 μmol), triethylsilane (not used in this example), trifluoroacetic acid (350 μl, 2.3 mmol), acetic anhydride (36 μl, 390 μmol) and N,N-diisopropylethylamine (130 μl, 770 μmol) gave the titled compound (28.0 mg, 95% purity, 15% yield) after preparative HPLC (basic method).

LC-MS (LC-MS METHOD 2): Rₜ=1.10 min; MS (ESIpos): m/z=501 [M+H]⁺

¹H-NMR (400 MHz, DMSO-d6) δ[ppm]: 1.531 (6.38), 1.549 (6.64), 2.105 (6.19), 2.145 (10.75), 2.336 (1.45), 2.371 (11.13), 2.374 (11.10), 2.394 (1.07), 2.453 (11.08), 2.518 (16.00), 2.523 (12.48), 2.613 (6.76), 2.679 (1.39), 3.658 (0.52), 3.720 (0.88), 3.744 (1.02), 3.765 (0.85), 3.814 (0.53), 3.833 (0.51), 3.860 (0.67), 3.891 (0.54), 3.903 (0.49), 3.967 (0.71), 4.019 (0.48), 4.130 (0.72), 4.174 (1.16), 4.347 (0.96), 4.366 (1.76), 4.379 (1.31), 4.392 (0.70), 4.404 (0.54), 5.688 (0.84), 5.703 (1.05), 5.720 (0.86), 7.339 (0.91), 7.359 (1.93), 7.377 (1.15), 7.540 (2.65), 7.559 (2.24), 7.738 (2.24), 7.758 (2.07), 8.670 (1.37), 8.683 (2.60), 8.693 (3.39).

### Example 69

### 1-{6-[4-({(1R)-1-[3-(difluoromethyl)-2-fluorophenyl]ethyl}amino)-2-methyl-7-(trifluoromethyl)pyrido[2,3-d]pyrimidin-6-yl]-2,6-diazaspiro[3.3]heptan-2-yl}ethan-1-one

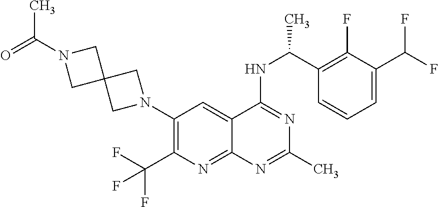 [SMILES: CC(=O)N1CC2(C1)CN(c1cc3c(N[C@H](C)c4cccc(C(F)F)c4F)nc(C)nc3nc1C(F)(F)F)C2]

Using the method described for Example 24, Intermediate 33 (tert-butyl 6-[4-({(1R)-1-[3-(difluoromethyl)-2-fluorophenyl]ethyl}amino)-2-methyl-7-(trifluoromethyl)pyrido[2,3-d]pyrimidin-6-yl]-2,6-diazaspiro[3.3]heptane-2-carboxylate, 40.0 mg, 67.0 μmol), triethylsilane (1.1 μl, 6.7 μmol), trifluoroacetic acid (100 μl, 1.3 mmol), acetic anhydride (7.0 μl, 74 μmol) and N,N-diisopropylethylamine (26 μl, 150 μmol) gave the titled compound (17.0 mg, 95% purity, 45% yield) after preparative HPLC (basic method).

LC-MS (LC-MS METHOD 2): Rₜ=1.18 min; MS (ESIpos): m/z=540 [M+H]⁺

¹H-NMR (400 MHz, DMSO-d6) δ[ppm]: 1.232 (0.46), 1.627 (4.72), 1.644 (4.72), 1.763 (14.40), 2.332 (2.19), 2.341 (16.00), 2.518 (7.60), 2.523 (5.44), 2.673 (1.72), 2.678 (0.75), 4.059 (4.56), 4.217 (0.51), 4.242 (5.61), 4.268 (0.50), 4.343 (4.70), 5.775 (0.73), 5.793 (1.15), 5.810 (0.72), 7.106 (1.12), 7.242 (2.39), 7.284 (0.82), 7.303 (1.81), 7.322 (1.03), 7.378 (0.98), 7.502 (0.63), 7.518 (1.01), 7.537 (0.51), 7.646 (0.56), 7.664 (1.03), 7.683 (0.51), 7.993 (3.36), 8.808 (1.18), 8.825 (1.12).

### Example 70

### 1-{6-[4-({(1R)-1-[3-(1,1-difluoro-2-hydroxy-2-methylpropyl)-2-fluorophenyl]ethyl}amino)-2-methyl-7-(trifluoromethyl)pyrido[2,3-d]pyrimidin-6-yl]-2,6-diazaspiro[3.3]heptan-2-yl}ethan-1-one

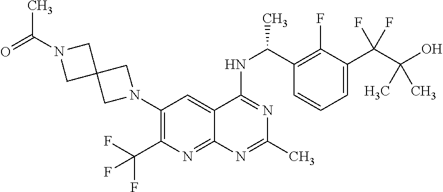 [SMILES: CC(=O)N1CC2(C1)CN(c1cc3c(N[C@H](C)c4cccc(C(F)(F)C(C)(C)O)c4F)nc(C)nc3nc1C(F)(F)F)C2]

Using the method described for Example 24, Intermediate 34 (tert-butyl 6-[4-{[(1R)-1-(3-{1,1-difluoro-2-methyl-2-[(triethylsilyl)oxy]propyl}-2-fluorophenyl)ethyl]amino}-2-methyl-7-(trifluoromethyl)pyrido[2,3-d]pyrimidin-6-yl]-2,6-diazaspiro[3.3]heptane-2-carboxylate, 80.0 mg, 104 μmol), triethylsilane (1.7 μl, 10 μmol), trifluoroacetic acid (160 μl, 2.1 mmol), acetic anhydride (11 μl, 110 μmol) and N,N-diisopropylethylamine (40 μl, 230 μmol) gave the titled compound (13.5 mg, 95% purity, 21% yield) after preparative HPLC (basic method).

LC-MS (LC-MS METHOD 2): Rₜ=1.12 min; MS (ESIpos): m/z=598 [M+H]⁺

¹H-NMR (400 MHz, DMSO-d6) δ[ppm]: 1.206 (6.44), 1.232 (7.09), 1.601 (4.56), 1.619 (4.55), 1.764 (14.21), 2.328 (16.00), 2.518 (9.19), 2.523 (6.27), 2.660 (0.69), 2.665 (1.56), 2.669 (2.20), 2.673 (1.56), 2.678 (0.69), 4.060 (4.67), 4.220 (0.48), 4.244 (6.71), 4.268 (0.50), 4.345 (4.76), 5.346 (6.83), 5.772 (0.75), 5.789 (1.16), 5.807 (0.73), 7.204 (0.73), 7.224 (1.73), 7.243 (1.12), 7.310 (0.77), 7.328 (1.13), 7.343 (0.53), 7.566 (0.61), 7.581 (1.09), 7.597 (0.56), 7.999 (3.33), 8.799 (1.25), 8.817 (1.20).

### Example 71

### 1-{6-[2-methyl-4-({(1R)-1-[2-methyl-3-(trifluoromethyl)phenyl]ethyl}amino)-7-(trifluoromethyl)pyrido[2,3-d]pyrimidin-6-yl]-2,6-diazaspiro[3.3]heptan-2-yl}ethan-1-one

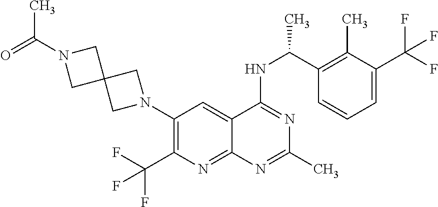 [SMILES: CC(=O)N1CC2(C1)CN(c1cc3c(N[C@H](C)c4cccc(C(F)(F)F)c4C)nc(C)nc3nc1C(F)(F)F)C2]

Using the method described for Example 24, Intermediate 35 (tert-butyl 6-[2-methyl-4-({(1R)-1-[2-methyl-3-(trifluoromethyl)phenyl]ethyl}amino)-7-(trifluoromethyl)pyrido[2,3-d]pyrimidin-6-yl]-2,6-diazaspiro[3.3]heptane-2-carboxylate, 20.0 mg, 32.8 μmol), triethyl silane (0.52 μl, 3.3 μmol), trifluoroacetic acid (50 μl, 660 μmol), acetic anhydride (3.4 μl, 36 μmol) and N,N-diisopropylethylamine (13 μl, 72 μmol) gave the titled compound (11.0 mg, 95% purity, 58% yield) after preparative HPLC (basic method).

LC-MS (LC-MS METHOD 2): Rₜ=1.28 min; MS (ESIneg): m/z=551 [M−H]⁻

¹H NMR (400 MHz, DMSO-d₆) δ ppm 8.90 (d) 7.99 (s) 7.75 (d) 7.56 (d) 7.37 (t) 5.72 (t) 4.34 (s) 4.21-4.27 (m) 4.06 (s) 2.67 (dt) 2.61 (s) 2.52-2.52 (m) 2.32-2.34 (m) 1.76 (s) 1.59 (d).

### Experimental Section—Biological Assays

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

Biochemical Assay: hK-RasG12C Interaction Assay with hSOS1

This assay quantifies the equilibrium interaction of human SOS1 (SOS1) with human K-Ras^{G12C} (K-RasG12C). Detection of the interaction is achieved by measuring homogenous time-resolved fluorescence resonance energy transfer (HTRF) from antiGST-Europium (FRET donor) bound to GST-K-RasG12C to anti-6His-XL665 bound to His-tagged hSOS1 (FRET-acceptor).

The assay buffer containes 5 mM HEPES pH 7.4 (Applichem), 150 mM NaCl (Sigma), 10 mM EDTA (Promega), 1 mM DTT (Thermofisher), 0.05% BSA Fraction V, pH 7.0, (ICN Biomedicals), 0.0025% (v/v) Igepal (Sigma) and 100 mM KF (FLUKA).

The expression and purification of N-terminal GST-tagged K-RasG12C and N-terminal His-tagged SOS1 is described below. Concentrations of protein batches used are optimized to be within the linear range of the HTRF signal. A Ras working solution is prepared in assay buffer containing typically 10 nM GST-hK-RasG12C and 2 nM antiGST-Eu(K) (Cisbio, France). A SOS1 working solution is prepared in assay buffer containing typically 20 nM His-hSOS1 and 10 nM anti-6His-XL665 (Cisbio, France). An inhibitor control solution is prepared in assay buffer containing 10 nM anti-6His-XL665 without SOS1.

Fifty nl of a 100-fold concentrated solution of the test compound in DMSO are transferred into a black microtiter test plate (384 or 1536, Greiner Bio-One, Germany).

For this, either a Hummingbird liquid handler (Digilab, MA, USA) or an Echo acoustic system (Labcyte, CA, USA) is used.

All steps of the assay are performed at 20° C. A volume of 2.5 μl of the Ras working solution is added to all wells of the test plate using a Multidrop dispenser (Thermo Labsystems). After 2 min preincubation, 2.5 μl of the SOS1 working solution are added to all wells except for those wells at the side of the test plate that are subsequently filled with 2.5 μl of the inhibitor control solution. After 60 min incubation the fluorescence is measured with a Pherastar (BMG, Germany) using the HTRF module (excitation 337 nm, emission 1: 620 nm, emission 2: 665 nm).

The ratiometric data (emission 2 divided by emission 1) are normalized using the controls (DMSO=0% inhibition, inhibition control wells with inhibitor control solution=100% inhibition). Compounds are tested in duplicates at up to 11 concentrations (for 20 μM, 5.7 μM, 1.6 μM, 0.47 μM, 0.13 μM, 38 nM, 11 nM, 3.1 nM, 0.89 nM, 0.25 nM and 0,073 nM). IC50 values are calculated by 4-Parameter fitting using a commercial software package (Genedata Screener, Switzerland).

| | K-RasG12C - SOS |
|---|---|
| | interaction assay |
| Example | IC₅₀ - [mol/l] (mean) |
| 1 | 4.09E−7 |
| 2 | 3.40E−8 |
| 3 | 1.39E−8 |
| 4 | 1.63E−8 |
| 5 | 4.24E−9 |
| 6 | 2.51E−7 |
| 7 | 1.80E−7 |
| 8 | 1.57E−8 |
| 9 | 6.43E−8 |
| 10 | 3.55E−9 |
| 11 | 1.76E−8 |
| 12 | 4.06E−9 |
| 13 | 6.35E−9 |
| 14 | 5.15E−9 |
| 15 | 1.06E−8 |
| 16 | 1.75E−8 |
| 17 | 3.56E−9 |
| 18 | 5.74E−9 |
| 19 | 8.25E−9 |
| 20 | 3.48E−7 |
| 21 | 9.49E−7 |
| 22 | 8.05E−7 |
| 23 | 2.64E−6 |
| 24 | 2.80E−9 |
| 25 | 5.78E−9 |
| 26 | 5.75E−9 |
| 27 | 7.96E−9 |
| 28 | 9.40E−9 |
| 29 | 2.56E−8 |
| 30 | 3.80E−8 |
| 31 | 1.19E−8 |
| 32 | 6.64E−8 |
| 33 | 2.79E−8 |
| 34 | 1.30E−8 |
| 35 | 7.71E−9 |
| 36 | 6.46E−9 |
| 37 | 8.47E−9 |
| 38 | 4.63E−8 |
| 39 | 1.62E−8 |
| 40 | 1.61E−8 |
| 41 | 2.30E−8 |
| 42 | 2.89E−8 |
| 43 | 5.20E−9 |
| 44 | 8.43E−9 |
| 45 | 2.11E−8 |
| 46 | 2.70E−9 |
| 47 | 4.00E−9 |
| 48 | 9.95E−9 |
| 49 | 3.31E−9 |
| 50 | 2.93E−9 |
| 51 | 9.79E−9 |
| 52 | 4.48E−9 |
| 53 | 3.34E−9 |
| 54 | 3.52E−9 |
| 55 | 4.46E−9 |
| 56 | 1.41E−8 |
| 57 | 9.81E−9 |
| 58 | 1.59E−8 |
| 59 | 7.23E−9 |
| 60 | 3.41E−6 |
| 61 | 2.53E−9 |
| 62 | 1.45E−8 |
| 63 | 8.66E−9 |
| 64 | 4.35E−9 |
| 65 | 5.72E−9 |
| 66 | 3.18E−9 |
| 67 | 6.23E−9 |
| 68 | 2.93E−9 |
| 69 | 2.95E−8 |
| 70 | 5.47E−9 |
| 71 | 5.78E−9 |

1. A compound of formula (I) 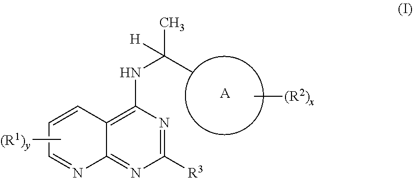
in which
A is selected from the group consisting of phenyl, naphthyl, heteroaryl and 9-10 membered bicyclic heterocyclyl;
R¹ is selected from
—H; or
-L-M, wherein
L is selected from
a single bond;
—C(R^{a})(R_{b})—;
—C(═O)—;
—S(═O)₂—;
—C(═O)—NR^{a}—; or
—S(═O)₂—NR^{a}—; and
R^{a} and R_{b} independently can be
—H;
C₁₋₆-alkyl, optionally substituted with a halogen or —OH;
C₃₋₈-cycloalkyl, optionally substituted with a halogen or —OH; or
or R^{a} and R_{b} together with the carbon atom they are attached to form a C₃₋₈-cycloalkyl or 4 to 6 membered heterocycloalkyl; and
M is selected from
C₁₋₆-alkyl; C₂₋₆-alkenyl; C₂₋₆-alkinyl; C₁₋₆-alkoxy; C₃₋₈-cycloalkyl; 4-6 membered heterocycloalkyl; phenyl; heteroaryl in which the C₁₋₆-alkyl; C₂₋₆-alkenyl; C₂₋₆-alkinyl; C₁₋₆-alkoxy; C₃₋₈-cycloalkyl; 4-6 membered heterocycloalkyl; phenyl and heteroaryl are all optionally substituted by one or more, identical or different Rₘ selected from
—OH, halogen, —CN; —C₁₋₆-alkyl; —C₃₋₆-cycloalkyl; —NRₙRₙ; —NRₙ—C(═O)—Rₙ; —NRₙ—S(═O)₂—Rₙ, —O—C₁₋₆-alkyl; —SRₙ; —S(O)—Rₙ, —S(O)₂—Rₙ or the bivalent oxo substituent, while the oxo substituent may only be a substituent in a non-aromatic ring and in which
each Rₙ is identical or different and independently selected from C₁₋₆-alkyl or C₃₋₈-cycloalkyl;
C₁₋₆-haloalkyl substituted with a 3 to 10 membered heterocyclyl; 3 to 10 membered heterocyclyl substituted with hydroxy, halogen, —NH₂, —SO₂—C₁₋₆-alkyl and the bivalent oxo-substituent, while the oxo-substituent may only be a substituent in a non-aromatic ring;
y is selected from 1 or 2;
R² is each independently selected from the group consisting of
C₁₋₆-alkyl;
C₁₋₆-haloalkyl;
C₂₋₆-alkenyl;
C₂₋₆-alkynyl;
C₃₋₈-cycloalkyl;
C₄₋₈-cycloalkenyl;
3-10 membered heterocyclyl;
phenyl and
heteroaryl;
wherein the C₁₋₆-alkyl, C₁₋₆-haloalkyl, C₂₋₆-alkenyl, C₂₋₆-alkynyl, C₃₋₈-cycloalkyl, C₄₋₈-cycloalkenyl, 3-10 membered heterocyclyl, phenyl and heteroaryl are all optionally substituted by one or more, identical or different R_{c} and/or R_{d};
in which each R_{c} is independently selected from the group consisting of halogen, —CN, —C(═O)R_{d}, —C(═O)OR_{d}, —C(O)NR_{d}R_{d}, —NR_{d}R_{d}, —OR_{d}, —S(═O)₂—R_{d}, —S(═O)₂—NR_{d}R_{d}, —NH—C(═O)—R_{d}, —N(CH₃)—C(═O)—R_{d}, —N(C₁₋₆-alkyl)C(═O)—R_{d}, —NH—C(═O)OR_{d}, —N(CH₃)—C(═O)OR_{d}, —N(C₁₋₆-alkyl)-C(═O)OR_{d} and —NR_{d}—S(═O)₂—R_{d}; and in which each R_{d} is independently selected from the group consisting of hydrogen, C₁₋₆-alkyl, C₁₋₆-haloalkyl, C₂₋₆-alkenyl, C₂₋₆-alkynyl, C₃₋₈-cycloalkyl, C₄₋₈-cycloalkenyl, 3-10 membered heterocyclyl, phenyl and heteroaryl, wherein the C₁₋₆-alkyl, C₁₋₆-haloalkyl, C₂₋₆-alkenyl, C₂₋₆-alkynyl, C₃₋₈-cycloalkyl, C₄₋₈-cycloalkenyl, 3-10 membered heterocyclyl, phenyl and heteroaryl are all optionally substituted by one or more, identical or different Rₑ and/or R_{f};
in which each Rₑ is independently selected from the group consisting of halogen, —CN, —C(═O)—R_{f}, —C(═O)OR_{f}, —C(═O) —NR_{f}R_{f}, —NR_{f}R_{f}, —OR_{f}, —S(═O)₂—R_{f}, —S(═O)₂NR_{f}R_{f}, —NHC(═O)R_{f}, —N(C₁₋₄ alkyl)C(═O) R_{f}, —NHC(═O)OR_{f} and —N(C₁₋₄ alkyl)C(═O)OR_{f}; and
in which each R_{f} is independently selected from the group consisting of hydrogen, C₁₋₆-alkyl, C₁₋₆-haloalkyl, C₂₋₆-alkenyl, C₂₋₆-alkynyl, C₃₋₈-cycloalkyl, C₄₋₈-cycloalkenyl, 3-10 membered heterocyclyl, phenyl and heteroaryl;
x is selected from 1 or 2
R³ is selected from
—H or —CH₃;
or a stereoisomer, a tautomer, an N-oxide, a hydrate, a solvate, or a salt thereof, or a mixture of same.

2. The compound according to claim 1 of formula (II) 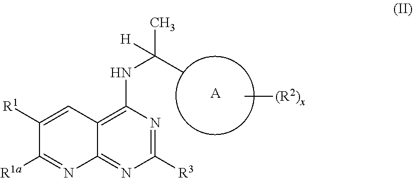
wherein
A is phenyl;
R¹ is selected from halogen, 5 to 10 membered mono or bicyclic heterocycloalkyl or heterocycloalkenyl with one or 2 nitrogen as heteroatoms and substituted by —CH₃, —C(═O)—CH₃ or —NH—C(═O)—CH₃,
R^{1a} is selected from hydrogen, —CH₃, CF₃ or —OCH₃;
R² is selected from hydrogen, halogen or C₁₋₆-alkyl optionally one or more time substituted by halogen and/or hydroxyl;
x is selected from 1 or 2 and
R³ is selected from hydrogen or —CH₃;
or a stereoisomer, a tautomer, an N-oxide, a hydrate, a solvate, or a salt thereof, or a mixture of same.

3. The compound according to claim 2 wherein
A is phenyl;
R¹ is selected from halogen, 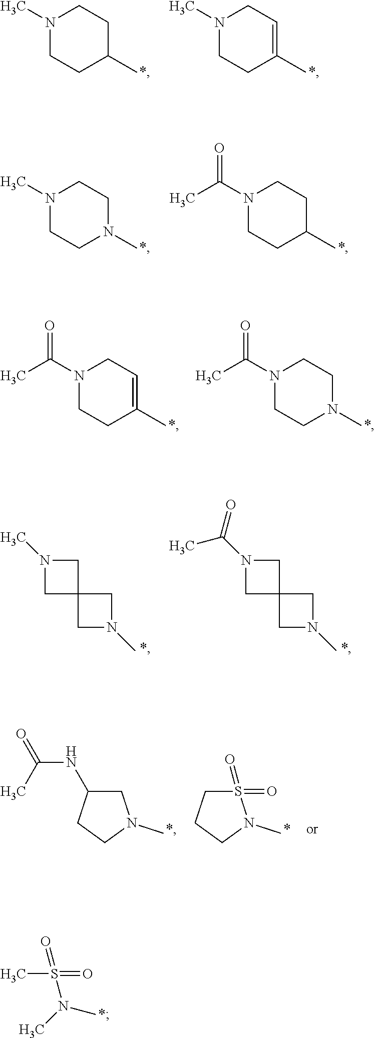
R^{1a} is selected from hydrogen, —CH₃, CF₃ or —OCH₃;
R² is each independently selected from —H, —CH₃, —F, —CF₃ or —CF₂—C(CH₃)₂—OH;
R³ is selected from hydrogen or —CH₃;
or a stereoisomer, a tautomer, an N-oxide, a hydrate, a solvate, or a salt thereof, or a mixture of same.

4. The compound according to claim 1 of formula (III) 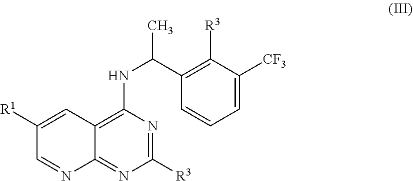
wherein
R¹ is selected from the group consisting of —Br, 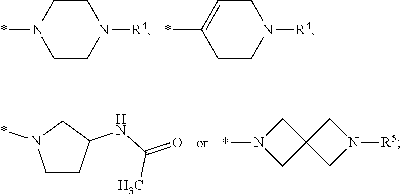
R³ is selected from the group consisting of —H and —CH₃;
R⁴ is selected from the group consisting of —CH₃ and —C(═O)—CH₃ and
R⁵ is selected from the group consisting of —C(═O)—CH₃ and —C(═O)OC(CH₃)₃ or a stereoisomer, a tautomer, an N-oxide, a hydrate, a solvate, or a salt thereof, or a mixture of same.

5. A compound according to claim 1, which is selected from the group consisting of:
6-bromo-N-{(1R)-1-[3-(trifluoromethyl)phenyl]ethyl}pyrido[2,3-d]pyrimidin-4-amine
N-{(3R)-1-[4-({(1R)-1-[3-(trifluoromethyl)phenyl]ethyl}amino)pyrido[2,3-d]pyrimidin-6-yl]pyrrolidin-3-yl}acetamide
6-(4-methylpiperazin-1-yl)—N-{(1R)-1-[3-(trifluoromethyl)phenyl]ethyl}pyrido[2,3-d]pyrimidin-4-amine
1-{4-[4-({(1R)-1-[3-(trifluoromethyl)phenyl]ethyl}amino)pyrido[2,3-d]pyrimidin-6-yl]piperazin-1-yl}ethan-1-one
1-{4-[4-({(1R)-1-[3-(trifluoromethyl)phenyl]ethyl}amino)pyrido[2,3-d]pyrimidin-6-yl]-3,6-dihydropyridin-1(2H)-yl}ethan-1-one
6-bromo-2-methyl-N-{(1R)-1-[2-methyl-3-(trifluoromethyl)phenyl]ethyl}pyrido[2,3-d]pyrimidin-4-amine
tert-butyl 6-[2-methyl-4-({(1R)-1-[2-methyl-3-(trifluoromethyl)phenyl]ethyl}amino)pyrido[2,3-d]pyrimidin-6-yl]-2,6-diazaspiro[3.3]heptane-2-carboxylate
1-{6-[2-methyl-4-({(1R)-1-[2-methyl-3-(trifluoromethyl)phenyl]ethyl}amino)pyrido[2,3-d]pyrimidin-6-yl]-2,6-diazaspiro[3.3]heptan-2-yl}ethan-1-one
1-{4-[4-({(1R)-1-[3-(trifluoromethyl)phenyl]ethyl}amino)pyrido[2,3-d]pyrimidin-6-yl]piperidin-1-yl}ethan-1-one
1-{4-[4-({(1R)-1-[2-methyl-3-(trifluoromethyl)phenyl]ethyl}amino)pyrido[2,3-d]pyrimidin-6-yl]piperazin-1-yl}ethan-1-one
N-{(3R)-1-[4-({(1R)-1-[2-methyl-3-(trifluoromethyl)phenyl]ethyl}amino)pyrido[2,3-d]pyrimidin-6-yl]pyrrolidin-3-yl}acetamide
1-{4-[4-({(1R)-1-[2-methyl-3-(trifluoromethyl)phenyl]ethyl}amino)pyrido[2,3-d]pyrimidin-6-yl]-3,6-dihydropyridin-1(2H)-yl}ethan-1-one
1-{6-[2-methyl-4-({(1R)-1-[2-methyl-3-(trifluoromethyl)phenyl]ethyl}amino)pyrido[2,3-d]pyrimidin-6-yl]-2,6-diazaspiro[3.3]heptan-2-yl}ethan-1-one-hydrogen chloride (1/1)
1-{(1S,4S)-5-[2-methyl-4-({(1R)-1-[2-methyl-3-(trifluoromethyl)phenyl]ethyl}amino)pyrido[2,3-d]pyrimidin-6-yl]-2,5-diazabicyclo[2.2.1]heptan-2-yl}ethan-1-one
2-methyl-6-(4-methylpiperazin-1-yl)—N-{(1R)-1-[2-methyl-3-(trifluoromethyl)phenyl]ethyl}pyrido[2,3-d]pyrimidin-4-amine
N-{(3R)-1-[2-methyl-4-({(1R)-1-[2-methyl-3-(trifluoromethyl)phenyl]ethyl}amino)pyrido[2,3-d]pyrimidin-6-yl]pyrrolidin-3-yl}acetamide
1-{4-[2-methyl-4-({(1R)-1-[2-methyl-3-(trifluoromethyl)phenyl]ethyl}amino)pyrido[2,3-d]pyrimidine-6-sulfonyl]piperazin-1-yl}ethan-1-one
N-{(3R)-1-[2,7-dimethyl-4-({(1R)-1-[2-methyl-3-(trifluoromethyl)phenyl]ethyl}amino)pyrido[2,3-d]pyrimidin-6-yl]pyrrolidin-3-yl}acetamide
1-{4-[2,7-dimethyl-4-({(1R)-1-[2-methyl-3-(trifluoromethyl)phenyl]ethyl}amino)pyrido[2,3-d]pyrimidin-6-yl]piperazin-1-yl}ethan-1-one
1-{6-[7-methoxy-2-methyl-4-({(1R)-1-[2-methyl-3-(trifluoromethyl)phenyl]ethyl}amino)pyrido[2,3-d]pyrimidin-6-yl]-2,6-diazaspiro[3.3]heptan-2-yl}ethan-1-one
1-{4-[7-methoxy-2-methyl-4-({(1R)-1-[2-methyl-3-(trifluoromethyl)phenyl]ethyl}amino)pyrido[2,3-d]pyrimidin-6-yl]-3,6-dihydropyridin-1(2H)-yl}ethan-1-one
1-{4-[7-methoxy-2-methyl-4-({(1R)-1-[2-methyl-3-(trifluoromethyl)phenyl]ethyl}amino)pyrido[2,3-d]pyrimidin-6-yl]piperidin-1-yl}ethan-1-one
2-[7-methoxy-2-methyl-4-({(1R)-1-[2-methyl-3-(trifluoromethyl)phenyl]ethyl}amino)pyrido[2,3-d]pyrimidin-6-yl]-1lambda⁶,2-thiazolidine-1,1-dione
1-{6-[4-({(1R)-1-[3-(1,1-difluoro-2-hydroxyethyl)-2-fluorophenyl]ethyl}amino)-2-methyl-7-(trifluoromethyl)pyrido[2,3-d]pyrimidin-6-yl]-2,6-diazaspiro[3.3]heptan-2-yl}ethan-1-one
1-{4-[2-methyl-4-({(1R)-1-[2-methyl-3-(trifluoromethyl)phenyl]ethyl}amino)pyrido[2,3-d]pyrimidin-6-yl]-3,6-dihydropyridin-1(2H)-yl}ethan-1-one
1-{4-[2-methyl-4-({(1R)-1-[2-methyl-3-(trifluoromethyl)phenyl]ethyl}amino)pyrido[2,3-d]pyrimidin-6-yl]-3,6-dihydropyridin-1(2H)-yl}ethan-1-one hydrogen chloride (1/1)
1-{7-[2-methyl-4-({(1R)-1-[2-methyl-3-(trifluoromethyl)phenyl]ethyl}amino)pyrido[2,3-d]pyrimidin-6-yl]-2-azaspiro[3.5]non-6-en-2-yl}ethan-1-one
1-{7-[2-methyl-4-({(1R)-1-[2-methyl-3-(trifluoromethyl)phenyl]ethyl}amino)pyrido[2,3-d]pyrimidin-6-yl]-2-azaspiro[3.5]nonan-2-yl}ethan-1-one
1-{6-[4-({(1R)-1-[3-(difluoromethyl)-2-fluorophenyl]ethyl}amino)-2-methylpyrido[2,3-d]pyrimidin-6-yl]-2,6-diazaspiro[3.3]heptan-2-yl}ethan-1-one 1-{3-[2-methyl-4-({(1R)-1-[2-methyl-3-(trifluoromethyl)phenyl]ethyl}amino)pyrido[2,3-d]pyrimidin-6-yl]-2,5-dihydro-1H-pyrrol-1-yl}ethan-1-one
1-{(3RS)-3-[2-methyl-4-({(1R)-1-[2-methyl-3-(trifluoromethyl)phenyl]ethyl}amino)pyrido[2,3-d]pyrimidin-6-yl]pyrrolidin-1-yl}ethan-1-one
6-methoxy-2-methyl-N-{(1R)-1-[2-methyl-3-(trifluoromethyl)phenyl]ethyl}pyrido[2,3-d]pyrimidin-4-amine
N-methyl-N-[2-methyl-4-({(1R)-1-[2-methyl-3-(trifluoromethyl)phenyl]ethyl}amino)pyrido[2,3-d]pyrimidin-6-yl]methanesulfonamide
2-[2-methyl-4-({(1R)-1-[2-methyl-3-(trifluoromethyl)phenyl]ethyl}amino)pyrido[2,3-d]pyrimidin-6-yl]-1lambda⁶,2-thiazolidine-1,1-dione
1-{4-[2-methyl-4-({(1R)-1-[2-methyl-3-(trifluoromethyl)phenyl]ethyl}amino)pyrido[2,3-d]pyrimidin-6-yl]piperazin-1-yl}ethan-1-one
1-{4-[2-methyl-4-({(1R)-1-[2-methyl-3-(trifluoromethyl)phenyl]ethyl}amino)pyrido[2,3-d]pyrimidin-6-yl]-4-oxo-4lambda⁵-piperazin-1-yl}ethan-1-one
1-{6-[4-({(1R)-1-[3-(difluoromethyl)-2-fluorophenyl]ethyl}amino)-2,7-dimethylpyrido[2,3-d]pyrimidin-6-yl]-2,6-diazaspiro[3.3]heptan-2-yl}ethan-1-one 6-methoxy-2,7-dimethyl-N-{(1R)-1-[2-methyl-3-(trifluoromethyl)phenyl]ethyl}pyrido[2,3-d]pyrimidin-4-amine
2-[4-({(1R)-1-[3-(difluoromethyl)-2-fluorophenyl]ethyl}amino)-2,7-dimethylpyrido[2,3-d]pyrimidin-6-yl]-1lambda⁶,2-thiazolidine-1,1-dione 1-{3-[2-methyl-4-({(1R)-1-[2-methyl-3-(trifluoromethyl)phenyl]ethyl}amino)pyrido[2,3-d]pyrimidin-6-yl]azetidin-1-yl}ethan-1-one
1-{4-[2-methyl-4-({(1R)-1-[2-methyl-3-(trifluoromethyl)phenyl]ethyl}amino)-7-(trifluoromethyl)pyrido[2,3-d]pyrimidin-6-yl]-3,6-dihydropyridin-1(2H)-yl}ethan-1-one
1-{4-[2-methyl-4-({(1R)-1-[2-methyl-3-(trifluoromethyl)phenyl]ethyl}amino)-7-(trifluoromethyl)pyrido[2,3-d]pyrimidin-6-yl]piperidin-1-yl}ethan-1-one
1-{4-[4-({(1R)-1-[3-(difluoromethyl)-2-fluorophenyl]ethyl}amino)-2-methylpyrido[2,3-d]pyrimidin-6-yl]-3,6-dihydropyridin-1(2H)-yl}ethan-1-one
1-{4-[4-({(1R)-1-[3-(difluoromethyl)-2-fluorophenyl]ethyl}amino)-2-methylpyrido[2,3-d]pyrimidin-6-yl]piperidin-1-yl}ethan-1-one
2-methyl-N-{(1R)-1-[2-methyl-3-(trifluoromethyl)phenyl]ethyl}-6-(pyrimidin-5-yl)pyrido[2,3-d]pyrimidin-4-amine
1-{4-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-3,6-dihydropyridin-1(2H)-yl}ethan-1-one
1-{4-[4-({(1R)-1-[3-(1,1-difluoro-2-hydroxy-2-methylpropyl)-2-fluorophenyl]ethyl}amino)-2-methylpyrido[2,3-d]pyrimidin-6-yl]piperidin-1-yl}ethan-1-one
6-[2-methyl-4-({(1R)-1-[2-methyl-3-(trifluoromethyl)phenyl]ethyl}amino)pyrido[2,3-d]pyrimidin-6-yl]-2,6-diazaspiro[3.3]heptane-2-carboxamide
1-{3-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-3,6-diazabicyclo[3.1.1]heptan-6-yl}ethan-1-one
1-{(1S,4S)-5-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-2,5-diazabicyclo[2.2.1]heptan-2-yl}ethan-1-one
1-{(1 R,4R)-5-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-2,5-diazabicyclo[2.2.1]heptan-2-yl}ethan-1-one
1,1-difluoro-1-{2-fluoro-3-[(1R)-1-{[2-methyl-6-(4-methylpiperazin-1-yl)pyrido[2,3-d]pyrimidin-4-yl]amino}ethyl]phenyl}-2-methylpropan-2-ol
1-{4-[4-({(1R)-1-[3-(1,1-difluoro-2-hydroxy-2-methylpropyl)-2-fluorophenyl]ethyl}amino)-2-methylpyrido[2,3-d]pyrimidin-6-yl]piperazin-1-yl}ethan-1-one
1-{6-[4-({(1R)-1-[3-(1,1-difluoro-2-hydroxy-2-methylpropyl)-2-fluorophenyl]ethyl}amino)-2-methylpyrido[2,3-d]pyrimidin-6-yl]-2,6-diazaspiro[3.3]heptan-2-yl}ethan-1-one
N-{(3R)-1-[4-({(1R)-1-[3-(1,1-difluoro-2-hydroxy-2-methylpropyl)-2-fluorophenyl]ethyl}amino)-2-methylpyrido[2,3-d]pyrimidin-6-yl]pyrrolidin-3-yl}acetamide
1-{4-[4-({(1R)-1-[3-(difluoromethyl)-2-fluorophenyl]ethyl}amino)-2,7-dimethylpyrido[2,3-d]pyrimidin-6-yl]-3,6-dihydropyridin-1(2H)-yl}ethan-1-one
1-{4-[4-({(1R)-1-[3-(difluoromethyl)-2-fluorophenyl]ethyl}amino)-2,7-dimethylpyrido[2,3-d]pyrimidin-6-yl]piperidin-1-yl}ethan-1-one
2-[2,7-dimethyl-4-({(1R)-1-[2-methyl-3-(trifluoromethyl)phenyl]ethyl}amino)pyrido[2,3-d]pyrimidin-6-yl]-1lambda⁶,2-thiazolidine-1,1-dione
2-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]-1lambda⁶,2-thiazolidine-1,1-dione
1-{6-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]-2,6-diazaspiro[3.3]heptan-2-yl}ethan-1-one
4-acetyl-1-[2-methyl-4-({(1R)-1-[2-methyl-3-(trifluoromethyl)phenyl]ethyl}amino)pyrido[2,3-d]pyrimidin-6-yl]piperazin-2-one
1-{4-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]-5-methyl-3,6-dihydropyridin-1(2H)-yl}ethan-1-one
1-{6-[2,7-dimethyl-4-({(1R)-1-[2-methyl-3-(trifluoromethyl)phenyl]ethyl}amino)pyrido[2,3-d]pyrimidin-6-yl]-2,6-diazaspiro[3.3]heptan-2-yl}ethan-1-one
1-{(1 S,4S)-5-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]-2,5-diazabicyclo[2.2.1]heptan-2-yl}ethan-1-one
1-{4-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]-3,6-dihydropyridin-1(2H)-yl}ethan-1-one
1-{4-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]piperidin-1-yl}ethan-1-one
1-{4-[4-({(1R)-1-[3-(1,1-difluoro-2-hydroxy-2-methylpropyl)-2-fluorophenyl]ethyl}amino)-2,7-dimethylpyrido[2,3-d]pyrimidin-6-yl]piperazin-1-yl}ethan-1-one
4-acetyl-1-[2,7-dimethyl-4-({(1R)-1-[2-methyl-3-(trifluoromethyl)phenyl]ethyl}amino)pyrido[2,3-d]pyrimidin-6-yl]piperazin-2-one
1-{6-[4-({(1R)-1-[3-(difluoromethyl)-2-fluorophenyl]ethyl}amino)-2-methyl-7-(trifluoromethyl)pyrido[2,3-d]pyrimidin-6-yl]-2,6-diazaspiro[3.3]heptan-2-yl}ethan-1-one
1-{6-[4-({(1R)-1-[3-(1,1-difluoro-2-hydroxy-2-methylpropyl)-2-fluorophenyl]ethyl}amino)-2-methyl-7-(trifluoromethyl)pyrido[2,3-d]pyrimidin-6-yl]-2,6-diazaspiro[3.3]heptan-2-yl}ethan-1-one
1-{6-[2-methyl-4-({(1R)-1-[2-methyl-3-(trifluoromethyl)phenyl]ethyl}amino)-7-(trifluoromethyl)pyrido[2,3-d]pyrimidin-6-yl]-2,6-diazaspiro[3.3]heptan-2-yl}ethan-1-one
or a stereoisomer, a tautomer, an N-oxide, a hydrate, a solvate, or a salt thereof, or a mixture of same.

6. A compound of formula (I) according to claim 1 for treatment or prophylaxis of a disease.

7. A pharmaceutical composition comprising a compound of formula (I) according to claim 1 and one or more pharmaceutically acceptable excipients.

8. A pharmaceutical combination comprising:
1. one or more first active ingredients, optionally one or more compounds of formula (I) according to claim 1, and
2. one or more further active ingredients, optionally anti-hyperproliferative and/or anti-cancer agents.

9. A product comprising a compound of formula (I) according to claim 1 for treatment or prophylaxis of a disease.

10. A product comprising a compound of formula (I) according to claim 1, said product comprising a medicament for treatment or prophylaxis of a disease.

11. A product according to claim 9, wherein the disease is a hyperproliferative disorder, optionally cancer.

12. A product comprising one or more SOS1 Inhibitors for treatment or prophylaxis of a disease, optionally cancer.
